(19) United States
(12) Patent Application Publication
(10) Pub. No.: US 2026/0364026 A1
(43) Pub. Date: Nov. 19, 2026

(54) TRANSDERMAL SYSTEMS HAVING POLYISOBUTYLENE AND METHODS OF MAKING AND USING THEM

(71) Applicant: Viatris Inc., Canonsburg, PA (US)

(72) Inventor: David P. Kanios, Palmetto Bay, FL (US)

(73) Assignee: Viatris Inc., Canonsburg, PA (US)

(21) Appl. No.: 19/668,397

(22) Filed: May 5, 2026

Related U.S. Application Data
(60) Provisional application No. 63/801,060, filed on May 6, 2025.
(60) Provisional application No. 63/802,308, filed on May 8, 2025.
(60) Provisional application No. 63/856,978, filed on Aug. 4, 2025.

(51) Int. Cl. A61K 9/70; A61K 9/00; A61K 31/57; A61P 15/18

(52) U.S. Cl. CPC A61K 9/7053; A61K 9/0014; A61K 31/57; A61P 15/18

(57) ABSTRACT

Transdermal systems, methods of use and making them are provided, the transdermal systems comprise a backing layer, a release liner, and a matrix disposed between the backing layer and the release liner, the matrix comprising an adhesive, the adhesive comprising a medium molecular weight polyisobutylene having an average molecular weight of 55,000 to 100,000, a high molecular weight polyisobutylene having an average molecular weight of 500,000 to 1,400,000 or both a medium molecular weight polyisobutylene having an average molecular weight of 55,000 to 100,000 and a high molecular weight polyisobutylene having an average molecular weight of 500,000 to 1,500,000.

### CROSS-REFERENCE TO RELATED APPLICATION

This application claims the benefit of pending applications U.S. Provisional Patent Application No. 63/856,978, filed Aug. 4, 2025; U.S. Provisional Patent Application No. 63/802,308, filed May 8, 2025; and U.S. Provisional Patent Application No. 63/801,060, filed May 6, 2025. The entire contents of each of the foregoing provisional applications are incorporated herein by reference.

### BACKGROUND

Transdermal systems include medicated adhesive patches that allow delivery of active pharmaceutical ingredients through the skin and into the blood stream at a relatively constant rate to the site of action until the transdermal system is removed from the skin. After the transdermal system is removed, the blood concentration decreases at a rate consistent with the pharmacokinetics of the active pharmaceutical ingredient. Because transdermal systems deliver the active pharmaceutical ingredient into the bloodstream, they can advantageously bypass the gastrointestinal system and the first-pass metabolism of the intestine and liver that occurs when medication is administered by the oral route.

Transdermal systems also offer greater patient comfort than, for example, the intravenous or intramuscular routes of administration that are associated with pain and discomfort from injection. Being easy to self-administer, transdermal systems do not require expert personnel for their placement, and generally only require that the site of application should be intact (not injured or wounded), clean, dry and relatively hairless. In the event of adverse reactions such as itching, irritation or erythema, the transdermal system can be easily removed to stop the drug delivery or placed at a different location where the skin is intact. This makes the transdermal system safe even when there is an intolerance to the administered active pharmaceutical ingredient.

To date, in the United States, there are 19 FDA approved transdermal systems in patch form, which are utilized in a wide range of conditions such as pregnancy prevention, hormone replacement, motion sickness, chronic pain, Alzheimer disease, Dementia associated with Parkinson disease, attention deficit hyperactivity disorder (ADHD) and others.

Of particular interest is the use of transdermal systems in patch form for the prevention of pregnancy, where the active pharmaceutical ingredients include a progestin and an estrogen in the patch to reduce or prevent pregnancy.

Currently, XULANE®, TWIRLA® and ZAFEMY® transdermal systems have been FDA approved for the prevention of pregnancy. XULANE® and ZAFEMY® both have a delivery rate of 35 mcg ethinyl estradiol (EE) and 150 mcg norelgestromin per day; and TWIRLA® has a delivery rate of 30 mcg ethinyl estradiol and 120 mcg levonorgestrel per day.

XULANE®, TWIRLA® and ZAFEMY® transdermal systems have warnings that use in women with a BMI≥30 can cause potentially a higher risk of venous thromboembolism events (VTE) compared to women with a lower BMI. Therefore, women with a BMI≥30 may not be able to use a transdermal system for birth control.

The goal with transdermal systems is to have the transdermal system adhere to the skin for the desired length of time after its application, maintain sufficient blood levels of the active ingredient (e.g., progestin and/or estrogen) and reduce unwanted spikes in blood levels of the active ingredient delivered by the transdermal system.

It would therefore be beneficial to provide transdermal systems (e.g., transdermal films or patches) that adhere to the skin for the desired length of time after their application and that maintain sufficient blood levels of the active ingredients over time that also avoid unwanted spikes in blood levels of the active ingredients delivered to the patient.

### SUMMARY

The present application provides transdermal systems (e.g., transdermal films or patches) that adhere to the skin for the desired length of time after their application and that maintain sufficient blood levels of the active ingredients over time and reduce unwanted spikes in blood levels of the active ingredients delivered to the patient.

The transdermal systems provided herein have integrity (e.g., minimal curling, wrinkling, delaminating and slippage) with minimal discomfort, irritation and sensitization both during use and after removal, and have minimal residual adhesive retention (or other components) after removal from the patient's skin.

In some embodiments, there is a transdermal system comprising a backing layer, a release liner, and a matrix disposed between the backing layer and the release liner, the matrix comprising an adhesive, the adhesive comprising a medium molecular weight polyisobutylene having an average molecular weight of 55,000 to 100,000, a high molecular weight polyisobutylene having an average molecular weight of 500,000 to 1,400,000 or both a medium molecular weight polyisobutylene having an average molecular weight of 55,000 to 100,000 and a high molecular weight polyisobutylene having an average molecular weight of 500,000 to 1,500,000.

In some embodiments, there is a transdermal system for releasing a contraceptive agent to a patient in need thereof, the transdermal system comprising a backing layer, a release liner, and a matrix disposed between the backing layer and the release liner, the matrix comprising an adhesive, the adhesive comprising a medium molecular weight polyisobutylene having an average molecular weight of 55,000 to 100,000, a high molecular weight polyisobutylene having an average molecular weight of 500,000 to 1,400,000 or both a medium molecular weight polyisobutylene having an average molecular weight of 55,000 to 100,000 and a high molecular weight polyisobutylene having an average molecular weight of 500,000 to 1,500,000, wherein the matrix is configured to release about 100 mcg per day to about 300 mcg per day of the contraceptive agent to the patient.

In some embodiments, there is a method of providing an active pharmaceutical ingredient to a patient in need thereof, the method comprising applying to the skin of the patient a transdermal system comprising a backing layer, a release liner, and a matrix disposed between the backing layer and the release liner, the matrix comprising an adhesive, the adhesive comprising a medium molecular weight polyisobutylene having an average molecular weight of 55,000 to 100,000, a high molecular weight polyisobutylene having an average molecular weight of 500,000 to 1,400,000 or both a medium molecular weight polyisobutylene having an average molecular weight of 55,000 to 100,000 and a high molecular weight polyisobutylene having an average molecular weight of 500,000 to 1,500,000 so as to release the active pharmaceutical agent through the skin of the patient.

In some embodiments, there is a method of making a transdermal system comprising a backing layer, a release liner, and a matrix disposed between the backing layer and the release liner, the method comprising applying to the matrix an adhesive, the adhesive comprising a medium molecular weight polyisobutylene having an average molecular weight of 55,000 to 100,000, a high molecular weight polyisobutylene having an average molecular weight of 500,000 to 1,400,000 or both a medium molecular weight polyisobutylene having an average molecular weight of 55,000 to 100,000 and a high molecular weight polyisobutylene having an average molecular weight of 500,000 to 1,500,000.

In some embodiments, there is a method of making a transdermal system, the method comprising mixing about 2.2 mg to about 5.3 mg of progestin per 7 to 14 cm² and applying it to a matrix, the matrix comprising an adhesive comprising a medium molecular weight polyisobutylene having an average molecular weight of 55,000 to 100,000, a high molecular weight polyisobutylene having an average molecular weight of 500,000 to 1,400,000 or both a medium molecular weight polyisobutylene having an average molecular weight of 55,000 to 100,000 and a high molecular weight polyisobutylene having an average molecular weight of 500,000 to 1,500,000, wherein the transdermal system is configured to release about 108 mcg per day to about 253 mcg per day of the progestin to a patient.

While multiple embodiments are disclosed, still other embodiments of the present disclosure will become apparent to those skilled in the art from the following detailed description. As will be apparent, the disclosure is capable of modifications in various obvious aspects, all without departing from the spirit and scope of the present disclosure. Accordingly, the detailed description is to be regarded as illustrative in nature and not restrictive.

### BRIEF DESCRIPTION OF THE FIGURES

In part, other aspects, features, benefits and advantages of the embodiments will be apparent with regard to the following description, appended claims, and accompanying figures.

FIG. 1 illustrates a graph of the overall adhesion rate across all adhesion/elegance studies for various transdermal systems (e.g., patches) including Unit A, a commercially marketed product, XULANE®, Unit B having a lot number 4001398, Unit C having a lot number 4001399, Unit D having a lot number 4001400, Unit E having a lot number 4001416, which has a unique polyisobutylene (PIB) combination of Medium Molecular Weight (MMW) PIB and High Molecular Weight (HMW) PIB, and Unit F having a lot number 4001439. Units B, C, D, E and F have no active ingredient in the system. Unit A did contain active ingredients in the system. Unit E had good softness and stiffness to the matrix.

FIG. 2 illustrates a graph of the adhesion rate of patches applied to an abdomen site in an adhesion/elegance study for various patches including Unit A, a commercially marketed product, XULANE®, Unit B having a lot number 4001398, Unit C having a lot number 4001399, Unit D having a lot number 4001400, Unit E having a lot number 4001416, and Unit F having a lot number 4001439. Units B, C, D, E and F have no active ingredient in the system. Unit A did contain active ingredients in the system.

FIG. 3 illustrates a graph of the adhesion rate of patches applied to an upper back site in an adhesion/elegance study for various patches including Unit A, a commercially marketed product, XULANE®, Unit B having a lot number 4001398, Unit C having a lot number 4001399, Unit D having a lot number 4001400, Unit E having a lot number 4001416, and Unit F having a lot number 4001439. Unit E has the most consistent wear rate over the 7 days compared to the other transdermal systems. Units B, C, D, E and F have no active ingredient in the system. Unit A did contain active ingredients in the system.

FIG. 4 illustrates a graph of the adhesion rate of patches applied to a buttock site in an adhesion/elegance study for various patches including Unit A, a commercially marketed product, XULANE®, Unit B having a lot number 4001398, Unit C having a lot number 4001399, Unit D having a lot number 4001400, Unit E having a lot number 4001416, and Unit F having a lot number 4001439. Unit E has the most consistent wear rate over the 7 days compared to the other transdermal systems as indicated by the higher linear line. Units B, C, D, E and F have no active ingredient in the system. Unit A did contain active ingredients in the system.

FIG. 5 illustrates a graph of norelgestromin (NE) in an amount of 4.86 mg and ethinyl estradiol (EE) in an amount of 0.264 mg in a transdermal system following a 7-day wear period on the sites of the abdomen, upper back, upper outer arm, and buttock in healthy adult female volunteers. It will be understood that any of these sites can be used to effectively deliver the NE and EE.

FIG. 6 illustrates a graph of norelgestromin in an amount of 4.86 mg and ethinyl estradiol in an amount of 0.264 mg in a transdermal system following a 7-day wear period on the abdomen in healthy adult female volunteers under normal, cool water, and whirlpool conditions.

FIG. 7 illustrates a graph of norelgestromin in an amount of 4.86 mg and ethinyl estradiol in an amount of 0.264 mg in a transdermal system following a 7-day wear period on the abdomen in healthy adult female volunteers under normal, sauna and exercise conditions.

FIG. 8 illustrates a graph of a single-dose comparative bioavailability and adhesion study from a transdermal system having norelgestromin in an amount of 4.86 mg and ethinyl estradiol in an amount of 0.264 mg in the transdermal system (14 cm²) and, a commercially marketed product, XULANE® Transdermal System (norelgestromin, 4.86 mg and ethinyl estradiol, 0.53 mg/14 cm²) following a 7-day wear period on the lower abdomen in healthy adult female volunteers. Unit E delivered a higher concentration of norelgestromin over time at about 166 ng*hr/mL, which is 10% higher than XULANE® Transdermal System.

FIG. 9 illustrates a graph of a single-dose comparative bioavailability and adhesion study of 4.86 mg of norelgestromin and 0.264 mg of ethinyl estradiol in a transdermal system (14 cm²) and, a commercially marketed product, EVRA® Canada following a 7-day wear period on the lower abdomen in healthy adult female volunteers.

FIG. 10 illustrates a graph of a single-dose bioavailability and adhesion study comparing three different patch sizes of norelgestromin transdermal systems following a 7-day wear period in healthy adult female volunteers.

FIG. 11 illustrates a graph of the single-dose pharmacokinetics and adhesive properties of a norelgestromin transdermal system, 4.86 mg/14 cm² following a single application on each of the proposed application sites, which included the lower abdomen, upper back, upper outer arm, and buttock for 7 days under normal conditions.

FIG. 12 illustrates a graph of a single-dose comparative bioavailability and adhesion study of norelgestromin transdermal systems, 2.43 mg/7 cm² following a 7-day wear period on the lower abdomen, upper back, upper outer arm, and buttock in healthy adult female volunteers.

FIG. 13 illustrates a graph of a single-dose comparative bioavailability and adhesion study of norelgestromin transdermal systems, 4.86 mg/14 cm² and XULANE® transdermal system (norelgestromin, 4.86 mg and ethinyl estradiol, 0.53 mg/14 cm²) following a 7-day wear period on the lower abdomen in healthy adult female volunteers.

FIG. 14 illustrates a graph of the single-dose pharmacokinetics and adhesive properties of a norelgestromin transdermal system, 4.86 mg/14 cm² following a single application on each of the proposed BMI Groups, which included BMI 19-25, BMI 35-40 and BMI>40 for 7 days under normal conditions.

FIG. 15 illustrates a schematic diagram of an embodiment of a transdermal system of the current application having a backing film or layer, a matrix film or layer disposed between the backing film or layer and a release liner. The release liner is shown with a slit for easy removal. The matrix having, among other things, an adhesive, which can be coated on a polymer, for example, a nonwoven polymer fabric.

FIG. 16 illustrates a single-dose comparative bioavailability study of norelgestromin 4.86 mg/ethinyl estradiol 0.264 mg transdermal system, norelgestromin 4.86 mg/ethinyl estradiol 0.40 mg transdermal system, XULANE® transdermal system (norelgestromin 4.86 mg/ethinyl estradiol 0.53 mg), and Ortho Evra transdermal system following a 7-day wear period in healthy adult female volunteers using a single adhesive system.

FIG. 17 illustrates a study to evaluate the multiple-dose pharmacokinetics of a commercially marketed product, Tri-Lo-Marzia™ Tablets versus a norelgestromin (NGMN) 4.86 mg/ethinyl estradiol (EE) 0.264 mg transdermal system following 3 patch applications over a 21-day period at two application sites. The release profiles are similar and show a near steady state release over time as opposed to the peaks and troughs seen with the immediate release tablets.

FIG. 18 presents a comparative analysis of the relationship between the Pearl Index and body mass index (BMI) for TWIRLA® and a transdermal system delivering norelgestromin at 190-240 μg/day and ethinyl estradiol at 17.5-20 μg/day using Unit E (containing those actives) and the adhesive described in Treatment E of the current application. The Pearl Index for TWIRLA® demonstrates a pronounced upward trend with increasing BMI, indicating reduced contraceptive effectiveness at higher BMI levels. In contrast, the transdermal system in an embodiment of the current application exhibits a relatively flat, slightly increasing Pearl Index across the BMI range. Notably, within the BMI cohort of 25-30 kg/m², the transdermal system shows a reduction in Pearl Index, suggesting improved or maintained efficacy in this population even with a lower estrogen dose.

FIG. 19 is a bar graph summarizing adhesion scores for a total of 39,790 patches that were applied by 1,272 participants, which showed improved adherence for Unit E that used the adhesive in Treatment E.

### DETAILED DESCRIPTION

### Definitions

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

The term “about” as used herein with regard to a stated value means within 10% of the stated value. In some embodiments, the term “about” may also refer to any number in between the significant digit of two values.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

It is noted that, as used in this specification and the appended claims, the singular forms “a,” “an,” and “the,” include plural referents unless expressly and unequivocally limited to one referent. Thus, for example, reference to “an active pharmaceutical ingredient” includes one, two, three or more active pharmaceutical ingredients.

As used in this specification and the appended claims, the term “or” is generally employed in its sense including “and/or” unless the content clearly dictates otherwise.

Unless defined otherwise, all technical and scientific terms used herein have the same meaning as is commonly understood by one of skill in the art to which the disclosure(s) belong. All patents, patent applications, published applications and publications, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

The use of terms of order or importance, including “first component” and “second component”, is to distinguish and identify individual elements and does not denote or imply a particular order or importance unless clearly indicated by context.

The term “active pharmaceutical ingredient” (API) or “active ingredient” as used herein refers to a chemical material or compound suitable for topical or transdermal administration and that induces a desired effect. The terms include API that are therapeutically effective, and/or prophylactically effective agents. The terms “active pharmaceutical ingredient” “active agent”, “active ingredient”, “drug” and “therapeutic agent” are used interchangeably herein.

The term “therapeutically effective amount” as used herein refers to the amount of an active agent that is nontoxic but sufficient to provide the desired therapeutic effect. The amount that is “effective” will vary from subject to subject, depending on the age and general condition of the individual, the particular active agent or agents, and the like as known to those skilled in the art.

The terms “transdermal” or “transdermal delivery” as used herein refer to administration of an active agent to a body surface of an individual so that the agent passes through the body surface, e.g., skin, and into the individual's blood stream (systemic circulation).

The terms “transdermal patch”, “transdermal device” and/or “transdermal system” all relate to a layered patch, device or system that provides transdermal delivery of an active agent therefrom. A transdermal system may comprise a backing layer, a matrix, among other things, containing the drug and the adhesive, and a release liner. In some embodiments, the transdermal system is a substantially non-aqueous, solid form, capable of conforming to the surface with which it comes into contact, and capable of maintaining such contact so as to facilitate topical application of the drug.

“Matrix” includes a repository or carrier for the therapeutic agent. The matrix can comprise a pressure-sensitive adhesive, which is adapted for direct application to a user's (e.g., a subject's) skin. The pressure-sensitive adhesive can include mixtures of the same or different polymers, such as polyisobutylene (PIB), of different molecular weights, wherein each resultant mixture is a pressure-sensitive adhesive. In some embodiments, the matrix comprises a pressure-sensitive adhesive at room temperature and has other desirable characteristics for adhesives used in the transdermal delivery arts. Such characteristics include good adherence to skin, ability to be peeled or otherwise removed without substantial trauma to the skin, retention of tack with aging, etc. In some embodiments, the matrix has a glass transition temperature (T_{g}), measured using a differential scanning calorimeter, of between about −70 °C and 0 °C.

As used in this specification and the appended claims, the term “Bioequivalence” and “Bioequivalent” means the absence of a significant difference in the rate and extent to which the active ingredient or active moiety in pharmaceutical equivalents or pharmaceutical alternatives become available at the site of drug action when administered at the same molar dose under similar conditions in an appropriately designed study.

“Bioavailability” means the rate and extent to which the active ingredient or active moiety is absorbed from a drug product and becomes available at the site of action.

“Patient” means any individual with or at risk of a specific health condition, whether or not he or she currently receives any therapy to prevent or treat that condition. Subjects can include humans, adult, and/or pediatric subjects. Subjects can also include primates such as chimpanzees, apes, orangutans and monkeys, rats, mice, cats, dogs, cows, horses, etc.

As used in this specification and the appended claims, the term “Pharmacokinetic parameters” refers to the assessment of drug behavior in the body by monitoring drug and metabolite concentrations in easily accessible physiological fluids like, for example, plasma and urine. These parameters provide an overall indication of drug kinetics, including absorption, distribution, metabolism, and excretion.

As used in this specification and the appended claims, the term “dry weight” refers to the mass of a substance after all its moisture content has been removed, and/or as it is measured according to United States Pharmacopeia (USP) standards or national formulary (NF) standards.

A “confidence interval” (CI) provides a range of values within a given confidence (e.g., 95%), including the accurate value of the statistical constraint within a targeted population. A CI provides a range with the lower bound and upper bound limits of a difference or association that would be plausible for a population.

The headings below are not meant to limit the disclosure in any way; embodiments under any one heading may be used in conjunction with embodiments under any other heading.

### Transdermal Systems

The present application provides transdermal systems (e.g., transdermal films or patches) that adhere to the skin for the desired length of time after their application and that maintain sufficient blood levels of the active ingredients over time and reduce unwanted spikes in blood levels of the active ingredients delivered to the patient.

The transdermal systems provided herein have integrity (e.g., minimal curling, wrinkling, delaminating and slippage) with minimal discomfort, irritation and sensitization both during use and after removal, and have minimal residual adhesive retention (or other components) after removal from the patient's skin.

In some embodiments, there is a transdermal system comprising a backing layer, a release liner, and a matrix disposed between the backing layer and the release liner, the matrix comprising an adhesive, the adhesive comprising a medium molecular weight polyisobutylene having an average molecular weight of 55,000 to 100,000, a high molecular weight polyisobutylene having an average molecular weight of 500,000 to 1,400,000 or both a medium molecular weight polyisobutylene having an average molecular weight of 55,000 to 100,000 and a high molecular weight polyisobutylene having an average molecular weight of 500,000 to 1,500,000.

The transdermal systems of the current application comprise, consist essentially of, or consist of at least three layers. Suitable configurations for transdermal systems for use in the present application include, for example, those transdermal systems described in U.S. patent application Ser. No. 18/553,368, filed Mar. 30, 2022, which published as U.S. Publication No. US 2024/0358655 and is assigned to Viatris Inc. The entire disclosure of this patent application is herein incorporated by reference into the present disclosure.

In the current application, the first layer can be a backing layer. The backing layer can be, for example, a flexible film or layer providing structural support to the transdermal system and can protect the matrix from the external environment until the release liner is removed and the transdermal system is applied to the skin. The matrix contains, among other things, the adhesive and one or more active ingredients and one or more inactive ingredients.

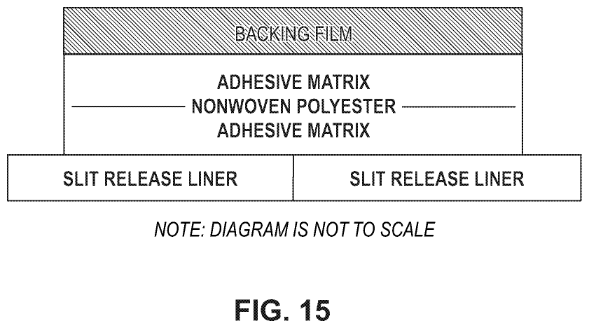

For example, the matrix can be a middle layer, or second layer disposed between the backing layer and the release liner as shown in FIG. 15. The matrix can contain not only the one or more active ingredients, but the polyisobutylene adhesive as described herein, and other inactive ingredients, such as for example, crospovidone, mineral oil, non-woven polyester fabric, oleyl alcohol and dipropylene glycol. Disposed on the matrix can be a release liner, which protects the matrix during storage and is removed prior to the application of the transdermal system to the skin. The release liner can be, for example, a transparent polyester film having a fluoropolymer coating on the side that is in contact with the matrix containing the adhesive.

The matrix can contain one or more active pharmaceutical ingredients that can be delivered from the matrix and through the skin of the patient once the transdermal system is applied to the skin. The one or more active pharmaceutical ingredients can include, for example, fertility drugs (e.g., clomiphene, letrozole, gonadotropins), scopolamine, nitroglycerin, clonidine, estradiol, fentanyl, nicotine, testosterone, donepezil, lidocaine, epinephrine, norethindrone, ethinyl estradiol, norelgestromin, levonorgestrel, oxybutynin, tetracaine, methylphenidate, selegiline, rotigotine, rivastigmine, opiates and opioids, e.g., alfentanil, allylprodine, alphaprodine, apomorphine, anileridine, apocodeine, benzylmorphine, bezitramide, buprenorphine, butorphanol, clonitazene, codeine, cyclorphan, cyprenorphine, desomorphine, dextromoramide, dextropropoxyphene, dezocine, diampromide, diamorphone, dihydrocodeine, dihydromorphine, dimenoxadol, eptazocine, ethylmorphine, etonitazene, etorphine, fentanyl, fencamfamine, fenethylline, hydrocodone, hydromorphone, hydroxymethylmorphinan, hydroxypethidine, isomethadone, levomethadone, levophenacylmorphan, levorphanol, lofentanil, mazindol, meperidine, metazocine, methadone, methylmorphine, modafinil, morphine, nalbuphene, necomorphine, normethadone, normorphine, opium, oxycodone, oxymorphone, pholcodine, profadol remifentanil, sufentanil, tramadol, corresponding derivatives, physiologically acceptable compounds, salts and bases or a combination thereof. In some embodiments, one or more active pharmaceutical ingredients can be in the transdermal system and include levonorgestrel, drospirenone, Selective Estrogen Receptor Modulators (SERMs) including tamoxifen, ospemifene, raloxifene, toremifene, bazedoxifene, clomifene, lasofoxifene, ormeloxifene or a combination thereof.

The above active pharmaceutical ingredients are made according to United States Pharmacopeia (USP) standards or national formulary (NF) standards and applied to the matrix. The delivery of the active ingredient from the transdermal system can be over 1 day, 2, 3, 4, 5, 6, 7, 8, 9, 10, 11, 12, 13, 14, 15, 16, 17, 18, 19, 20, 21, 22, 23, 24, 25, 26, 27, 28, 29, to 30 days. The transdermal system can be worn from 1 day to 30 days. In one particular embodiment, the transdermal system is worn for 1 week.

The third layer can be the release liner, which protects the adhesive layer during storage and is removed just prior to application. The release liner can be made from, for example, polyester, a silicone coated polyethylene terephthalate, a fluorocarbon, a fluorocarbon coated polyethylene terephthalate, and a fluorosilicone coated polyethylene terephthalate. In some embodiments, the release liner is made from a polyester film with a fluoropolymer coating on the side that is in contact with the middle adhesive layer.

Non-limiting examples of materials from which the backing layer may be composed include polyethylene terephthalate, various nylons, polypropylenes, polyesters, polyester/ethylene-vinyl acetate, metalized polyester films, polyvinylidene chloride, metal films such as aluminum foils, polyvinylidene fluoride films, and mixtures, copolymers or laminates thereof. Specific backing layers which may be utilized include Mediflex® 1200, Mediflex® 1501, Mediflex® 1502, Mediflex® 1503. Mediflex® 1510, Mediflex® 1511, and Mediflex® 1512 (each available from Mylan Technologies, Inc.), and Scotchpak® 1022, or Scotchpak® 1122 (available from 3M).

In one embodiment, the release liner is Scotchpak® 1022, which is a transparent, fluoropolymer-coated polyester film, which easily peels away from the pressure sensitive PIB adhesive that allows for clean separation from the adhesive surface it is applied to.

Other non-limiting materials for the backing layer include ethylene vinyl acetate films laminated to a polyester, ethylene vinyl acetate films laminated to a metallized polyester, MEDIFLEX® 1200, MEDIFLEX® 1501, MEDIFLEX® 1505, MEDIFLEX® 1201, MEDIFLEX® 1502 (all five MEDIFLEX® products being available from Mylan Technologies Inc., St. Albans, Vt., USA), DuPont polyester type S available from DuPont, Wilmington, Del., USA, Dow BLF® 2050 available from The Dow Chemical Company, Midland, Mich., USA, and 3M™ Scotchpak™ 1109, 3M™ Scotchpak™ 9723, 3M™ Scotchpak™ 9733, 3M™ Scotchpak™ 9735 and 3M™ Scotchpak™ 9730, which Scotchpak™ products are available from 3M of Minneapolis, Minn., USA.

Additional materials suited for the backing layer include polyethylene or polyolefin backings, such as MEDIFLEX® 1000, 3M™ CoTran™ 9722, and 3M™ CoTran™ 9720. 3M™ CoTran™ products are available from 3M of Minneapolis, Minn., USA.

In some embodiments of the present application, the backing layer comprises a film selected from MEDIFLEX® 1501 (which has an MVTR of 14 g/m²·24 hr), MEDIFLEX® 1000 (which has an MVTR of 7 g/m²·24 hr), available from Mylan Technologies Inc., Scotchpak™ 1109, Scotchpak™ 9730, Scotchpak™ 9732, Scotchpak™ 9733, Scotchpak™ 9735, as well as CoTran™ 9719, CoTran 9720, and CoTran 9726, available from 3M. The Scotchpak™ and CoTran films exhibit a relatively low Moisture Vapor Transmission Rate (MVTR) range of from about 0.5 to about 17 g/m²·24 hr.

In certain embodiments, the backing layer is comprised of ethylene vinyl acetate films laminated to a polyester, such as MEDIFLEX® 1501 from Mylan Technologies, Inc. MEDIFLEX® 1501 is a three layer structure which can be described from its external surface inwardly as follows: peach colored polyethylene/polyurethane adhesive/polyester.

In some embodiments, a suitable material that the matrix can be coated on in the middle layer can be sourced from Reemay® and can contain nonwoven spunbonded polyester. REEMAY® spunbonded polyester is a sheet structure of continuous filament polyester fibers that are randomly arranged, highly dispersed, and bonded at the filament junctions including Reemay® 2004, Reemay® 2006, Reemay® 2011, Reemay® 2014, Reemay® 2016, Reemay® 2024, Reemay® 2033, Reemay® 2040, Reemay® 2200, Reemay® 2214, Reemay® 2250, Reemay® 2075, and Reemay® 2070 with various denier per filament, basis weight, thickness and grab tensile. In one embodiment, Reemay® 2250 has a 2.2 denier per filament with 0.75 oz/yard², thickness of 6 mils and 10×8 grab tensile lbs (MD×XD).

### Progestin/Estrogen

In one embodiment of the present application, the transdermal system (e.g., film or patch) can be used for preventing ovulation in a woman. In some embodiments, the transdermal delivery system used for this method is a transdermal patch, comprising a backing and a matrix contacting the backing, the matrix comprising a mixture of norelgestromin and/or a low dose of an estrogen, and a pressure sensitive adhesive and the transdermal system is adapted to be in diffusional communication with the skin of the woman and to administer an ovulation-inhibiting amount of norelgestromin and the low dose estrogen to said skin.

In a particular embodiment, the transdermal system comprises a backing, and a matrix contacting the backing, the matrix comprising a polyisobutylene (PIB), the PIB comprising a PIB mixture, the PIB mixture comprising a medium molecular weight (MMW) PIB and a high molecular weight (HMW) PIB. The HMW PIB comprises a high viscosity PIB such as Oppanol® N100 (4-8 wt %) and/or Oppanol® N80 (0.5 to 5 wt %). The MMW PIB comprises a low viscosity PIB such as Oppanol® B10SFN (20-30 wt %) and/or Oppanol® B12SFN (1-15 wt %). The abbreviation SFN includes stabilizer free and does not contain a stabilizer (e.g., butylated hydroxytoluene (BHT)).

In another embodiment, the transdermal system comprises a backing, and a matrix contacting the backing, the matrix comprising a polyisobutylene (PIB), the PIB comprising a PIB mixture, the PIB mixture comprising at least two medium molecular weight (MMW) PIBs and at least two high molecular weight (HMW) PIBs. The HMW PIBs comprise a mixture of PIBs exemplified by Oppanol® N100 (5-10 wt %) and/or Oppanol® N80 (0.5 to 5 wt %). The MMW PIBs comprise a mixture of HMW PIBs exemplified by Oppanol® B10SFN (20-30 wt %) and/or Oppanol® B12SFN (1-15 wt %).

In some embodiments, the PIB mixture comprises a solvent comprising water, n-heptane, heptane, hexane, ethyl acetate, toluene, or a lower chain alcohol such as ethanol, propanol, or isopropanol.

In one particular embodiment, the PIBs comprise a mixture of at least two MMW PIBs and at least two HMW PIBs, where the MMW PIBs comprise Oppanol® N100 having a MW of about 1,550,000 (in an amount of about 5.06 wt %) and/or Oppanol® N80 having a MW of about 1,050,000 (in an amount of about 0.56 wt %). The HMW PIBs comprise Oppanol® B10SFN having a MW of about 53,000 (in an amount of about 26.1 wt %) or Oppanol® B12SFN having a MW of about 70,000 (in an amount of about 1.38 wt %).

In a particular embodiment, the transdermal system comprises a backing, and a matrix contacting the backing, the matrix comprising a polyisobutylene (PIB), the PIB comprising a PIB mixture, the PIB mixture comprising a first PIB having a medium molecular weight (MMW) less than 100,000 molecular weight (MW) and a second PIB having a high molecular weight (HMW) more than 100,000 MW, the PIB mixture having a MMW to HMW ratio from about 80:20 to about 90:10 such that the transdermal system has an adhesion rate greater than 90% after 7 days of application. In some embodiments, the matrix is configured to release about 5 mcg per day to about 350 mcg per day of the contraceptive agent to the patient. The contraceptive agent can be a progestin alone, an estrogen alone or a combination thereof. In another embodiment, the PIB mixture has a MMW to HMW ratio from about 55:45. In other embodiments, the PIB mixture has a MMW:HMW:HMW ratio of 55:22.5:22.5, respectively.

In various embodiments, the backing is in the form of a layer and is impermeable to progestin and/or estrogen. The matrix comprises an adhesive to permit contact with the skin of a patient and allow the progestin and/or estrogen to be released from the matrix through the skin of the patient.

Progestins which can be used in the transdermal system include one or more of norelgestromin, levonorgestrel, norgestrel, norgestimate, desogestrel, gestodene, norethindrone, norethynodrel, hydrogesterone, ethynodiol dicetate, hydroxyprogesterone caproate, medroxyprogesterone acetate, norethindrone acetate, progesterone, megestrol acetate, gestogen and certain others which are biocompatible, absorbable transdermally, including biocompatible derivatives of progestins which are transdermally absorbed, desirably such derivatives which are bioconvertible after transdermal absorption to the original progestin. The progestin and estrogen hormones should have high compatibility with each other.

The effective dose of norelgestromin for inhibiting ovulation normally varies in a range of from about 100, 105, 110, 115, 120, 125, 130, 140, 145, 150, 155, 160, 165, 170, 175, 180, 190, 200, 210, 220, 230, 240, 250, 260, 270, 280, 290, 300, 310, 320, 330, 340 to about 350 mcg/day, in some aspects, from about 125 to about 300 mcg/day, and in other aspects, from about 140 to about 200 mcg/day. In some embodiments, the amount of norelgestromin to deliver per day can be from about 190 mcg/day to about 200 mcg/day or to about 240 mcg/day. In some aspects, the norelgestromin can be micronized.

The effective dose of levonorgestrel for inhibiting ovulation normally varies in a range of about 50, 60, 70, 80, 90, 100, 110, 120, 130, 140, 150, 160, 170, 180, 190, 200, 210, 220, 230, 240, 250, 260, 270, 280, 290 to about 300 mcg/day, in some aspects, from about 100 to about 200 mcg/day, and in other aspects from about 100 to about 150 mcg/day.

The progestin (e.g., norelgestromin) can be used alone or the estrogen (e.g., ethinyl estradiol) can be used alone or the progestin and estrogen combined together in the matrix of the transdermal system.

The estrogen which can be used in the transdermal system include one or more of 17-β-estradiol and esters thereof such as estradiol valerate, estradiol cypionate, estradiol acetate, estradiol benzoate, and ethinyl estradiol (EE). EE is a preferred estrogen for use in combination with norelgestromin.

The effective dose of estrogen for inhibiting ovulation will depend upon the particular estrogen being used in the matrix. For instance, in some aspects, when the estrogen is ethinyl estradiol (EE), the dose will be at least 5 mcg/day, and in other aspects, from about 10 to 30 mcg/day. The patches will contain sufficient amounts of ethinyl estradiol to provide such daily doses for the intended patch wear time. Typically, such doses are about 5, 6, 7, 8, 9, 10, 11, 12, 13, 14, 15, 16, 17, 18, 19, 20, 21, 22, 23, 24, 25, 26, 27, 28, 29 to about 30 mcg/day, in various embodiments from about 10 to about 30 mcg/day, and in other embodiments from about 15 to about 27 mcg/day of ethinyl estradiol. In some aspects, the ethinyl estradiol can be micronized.

When a transdermal patch is worn for contraception, the patch will typically be placed on the skin on the first day of the menstrual cycle and replaced as needed until 21 days of wearing have elapsed. For instance, in the case of a 7-day patch, three patches will be required to deliver the drug(s) for the 21-day period. If desired, a placebo patch may be worn thereafter until the fifth day of the succeeding menstrual cycle. This regimen is repeated for each menstrual cycle.

The transdermal system of this application can be in the form of a patch or a film. Patches or films of this application comprise a matrix and are monolithic-type laminated structures. They comprise a matrix of the drug(s) admixed with a pressure sensitive adhesive and a backing. The matrix serves as both the drug reservoir and the means by which the patch or film is affixed to the skin. Prior to use, the transdermal system will also include an impermeable release liner layer. The release liner contacts the matrix and is configured to be removed from the matrix. By using the transdermal system of this application, the pregnancy of a woman patient is prevented where the woman patient has a body mass index (BMI) of less than 30 kg/m².

In some embodiments, each transdermal system of the present application contains two active pharmaceutical agents, such as norelgestromin and ethinyl estradiol, which are mixed in a pressure-sensitive adhesive matrix, which is designed to deliver one or more active agents transdermally.

The transdermal system of the current application allows delivery of, for example, about 108 to 253 mcg/day of progestin to the patient, while the delivery of other optional active agents of the transdermal delivery system, such as for example, estrogen are not interfered with. Thus, the patient can have consistent release of both progestin and estrogen, even though the dose of estrogen may be lower.

In some embodiments, the transdermal system of the present application is configured to deliver about 17.5 μg to about 20 μg of ethinyl estradiol per day and about 190 μg to about 240 μg of norelgestromin per day to a patient over a seven (7) day wear period. In some embodiments, the amount of ethinyl estradiol and norelgestromin delivered to the patient is determined by residual analysis of the worn transdermal system. For example, after the transdermal system has been worn for about seven days, the transdermal system is removed from the patient and subjected to a residual assay to measure the amount of ethinyl estradiol and the amount of norelgestromin remaining in the transdermal system. The amount delivered to the patient during the wear period can then be determined based on the difference between (i) the initial amount of ethinyl estradiol and norelgestromin present in the transdermal system prior to application and (ii) the residual amount of ethinyl estradiol and norelgestromin remaining in the transdermal system after the wear period.

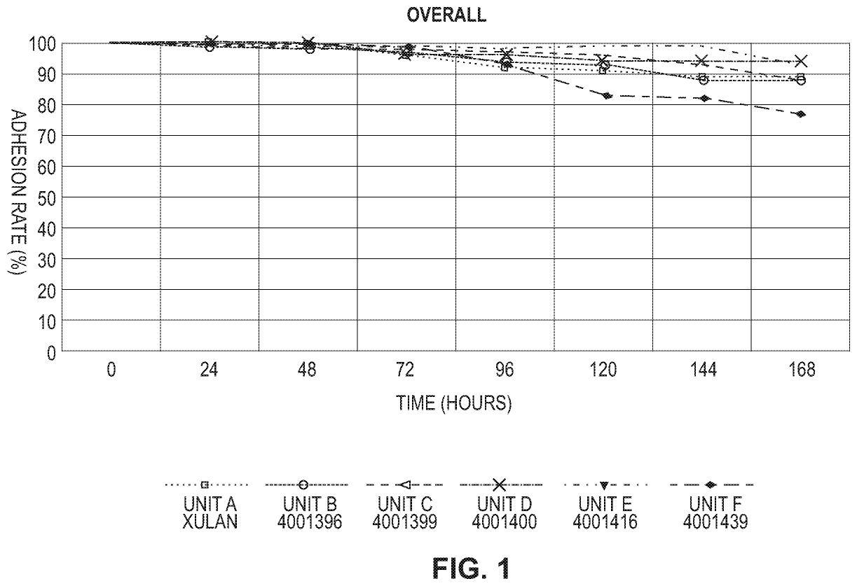

FIGS. 1-4 illustrate the graphs of the adhesion rate of various patches applied to various body sites under various conditions. The patches comprise various components as discussed herein. The backing of the transdermal system is impermeable to the drug and other components of the matrix and defines the top face surface of the patch. It may be made of a single layer or film of polymer or be a laminate of one or more polymer layers and a metal foil. Examples of polymers suitable for use in making backing films include without limitation polyvinylchloride, polyvinylidene chloride, polyolefins such as ethylene-vinyl acetate copolymers, polyethylene, and polypropylene, polyurethane, and polyesters such as polyethylene terephthalate. In many aspects, the backing is impermeable to both estrogen, for example, ethinyl estradiol and/or progestin, for example norelgestromin.

The pressure-sensitive adhesive of the matrix will normally be prepared from a solution of a unique blend of polyisobutylene (PIB). PIB adhesives with desired MW ranges can be made from a PIB source such as Oppanol® from BASF, Florham Park, New Jersey, as exemplified in e.g., U.S. Pat. No. 5,508,038. For example, in Table 1, PIB X is a PIB comprising a first component, which is both a medium molecular weight polyisobutylene, for example, having an average molecular weight of about 70,000, which can be obtained from BASF, Florham Park, New Jersey, in the product Oppanol® B-B12 (12N) and a high molecular weight polyisobutylene, for example, having an average molecular weight of about 1,050,000, which can be obtained from BASF, Florham Park, New Jersey, in the product Oppanol® N-80. The PIB, in some embodiments, can be stabilized with butylated hydroxytoluene at an average concentration of 500 ppm.

The PIB blend of the current application also utilizes a second component. For example, in Table 1, PIB Y is a PIB comprising a second component, which is both a medium molecular weight polyisobutylene, for example, having an average molecular weight of about 53,000, which can be obtained from BASF, Florham Park, New Jersey, in the product Oppanol® B-B10 (ION) and a high molecular weight polyisobutylene, for example, having an average molecular weight of about 1,550,000, which can be obtained from BASF, Florham Park, New Jersey, in the product Oppanol® N-100. The PIB, in some embodiments, can be stabilized with butylated hydroxytoluene at an average concentration of 500 ppm.

These two components can be mixed together in the amounts indicated in Table 1 with the other components indicated in Table 1 to form the matrix, which is applied to a polymer fabric, such as for example, non-woven polyester to form the medicated layer to the patch, which can be disposed between the backing and the release liner.

In some embodiments, polyisobutylene (PIB) adhesives are mixtures of at least one high molecular weight (HMW) PIB and at least one medium molecular weight (MMW) PIB. Such mixtures are described in the art, e.g., WO1991016085, published Oct. 31, 1991 to Alza Corporation. The entire disclosure is herein incorporated by reference. Each high molecular weight polyisobutylene may have an average molecular weight of 500,000 to 1.5 million, or from 750,000 to 1.2 million. Each low molecular weight polyisobutylene may have an average molecular weight of 40,000 to 85,000.

Weight average molecular weight includes, for example, the average molecular weight of the PIB, where larger molecules contribute more to the average due to their greater mass, essentially giving more “weight” to the larger molecules in the calculation; in the USP (United States Pharmacopeia), it is typically measured using size-exclusion chromatography (SEC), which separates molecules based on size, allowing for the calculation of the average molecular weight based on the distribution of molecule sizes in the sample, with larger molecules eluting first.

Suitable polyisobutylene adhesives for use in the current application include mixtures of Oppanol® N80 (HMW PIB), Oppanol® N100 (HMW PIB) and Oppanol® B12 (MMW PIB). In another embodiment, Oppanol® N100 (HMW PIB) or Oppanol® B100 (HMW PIB) and Oppanol® B10 (MMW PIB) may be used.

One specific adhesive to use, for example, includes PIB X as a first component, PIB Y as the second component for the adhesive mentioned Table 1 Treatment E.

For adhesives using a mixture of high and medium molecular weight PIBs, the dry weight ratio of medium molecular weight to high molecular weight PIB will normally range from 15:1, 14:1, 13:1, 12:1, 11:1, 10:1, 9:1, 8:1, 7:1, 6:1, 5:1, 4:1, 3:1, 2:1, 1:1. The molecular weights referred to herein are weight average molecular weight. Additionally, a mixture of adhesives may be used to achieve the desired adhesion and flow throughout the patch. The adhesives may be present in a dry weight ratio of from 15:1, 14:1, 13:1, 12:1, 11:1, 10:1, 9:1, 8:1, 7:1, 6:1, 5:1, 4:1, 3:1, 2:1, 1:1.

In some embodiments, polyisobutylene adhesives are multiple mixtures of high and medium molecular weight PIBS. For example, in some embodiments, a first mixture comprises Oppanol® N80, Oppanol® N100 and Oppanol® B10 and a second mixture comprises Oppanol® N100 and Oppanol® B10. In various embodiments, a mixture comprises MMW from about 40,000 to about 85,000 viscosity average molecular weight (Mv); or from about 53,000 to about 108,000 weight average molecular weight (Mw) and HMW from about 425,000 to about 2,600,000 viscosity average molecular weight Mv; or from about 565,000 to about 3,050,000 about 40,000 to about 85,000 viscosity average molecular weight (Mv); or from about 53,000 weight average molecular weight (Mw). In some embodiments, the Mw for MMW PIB is from about 50,000, 51,000, 52,000, 53,000, 54,000, 55,000, 56,000, 57,000, 58,000, 59,000, 60,000, 61,000, 62,000, 63,000, 64,000, 65,000, 66,000, 67,000, 68,000, 69,000, 70,000, 71,000, 72,000, 73,000, 74,000, 75,000, 76,000, 77,000, 78,000, 79,000, 80,000, 81,000, 82,000, 83,000, 84,000, 85,000, 86,000, 87,000, 88,000, 89,000, 90,000, 91,000, 92,000, 93,000, 94,000, 95,000, 96,000, 97,000, 98,000, 99,000, 100,000, 101,000, 102,000, 103,000, 104,000, 105,000, 106,000, 107,000, 108,000, 109,000, 110,000, to about 111,000. In some embodiments, the Mw for HMW PIB is from about 510,000, 560,000, 610,000, 660,000, 710,000, 760,000, 810,000, 860,000, 910,000, 960,000, 1,010,000, 1,060,000, 1,110,000, 1,160,000, 1,210,000, 1,260,000, 1,310,000, 1,360,000, 1,410,000, 1,460,000, 1,510,000, 1,560,000, 1,610,000, 1,660,000, 1,710,000, 1,760,000, 1,810,000, 1,860,000, 1,910,000, 1,960,000, 2,010,000, 2,060,000, 2,110,000, 2,160,000, 2,210,000, 2,260,000, 2,310,000, 2,360,000, 2,410,000, 2,460,000, 2,510,000, 2,560,000, 2,610,000, 2,660,000, 2,710,000, 2,760,000, 2,810,000, 2,860,000, 2,910,000, 2,960,000, 3,010,000, 3,060,000, 3,110,000, 3,160,000, 3,210,000, 3,260,000, to about 3,310,000.

In one embodiment, the first mixture and the second mixture of the PIB mixture are different combinations of one or more high molecular weight PIB and one or more medium molecular weight PIB. In one embodiment, the first mixture comprises polyisobutylene having from about 25, 26, 27, 28, 29, 30, 31, 32, 33, 34 to about 35% MMW. In some embodiments, the first mixture comprises polyisobutylene having from about 60, 61, 62, 63, 64, 65, 66, 67, 68, 69, 70, 71, 72, 73, 74, 75, 76, 77, 78, 79. 80, 81, 82, 83, 84, 85, 86, 87, 88, 89, 90, 91, 92, 93, 94, 95, 96, 97, 98 to about 99% MMW and from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 to about 40% HMW. In one embodiment, PIB comprises 84% MMW and 16% HMW. In another embodiment, PIB comprises a first mixture of 84% MMW and 16% HMW and a second mixture of 55% MMW, 22.5% of a first HMW and 22.5% of a second HMW. In various embodiments, 84% MMW comprises PIB having 53,000 Mw and 16% HMW, which comprises PIB having 1,110,000 Mw. In another embodiment, 84% MMW comprises PIB having 53,000 Mw and 16% HMW, which comprises PIB having 1,550,000 PIB. In one embodiment, the PIB comprises a first mixture of 55% MMW having 70,000 Mw, 22.5% first HMW having 1,550,000 Mw and 22.5% HMW having 1,050,000 Mw and a second mixture of 84% MMW having 53000 Mw and 16% HMW having 1,550,000 Mw. In some embodiments, the PIB comprises 75.6% of a first MMW PIB, 5.5% of a second MMW PIB, 16.65% of a first HMW PIB and 2.25% of a second HMW PIB. In some embodiments, the PIB comprises a first mixture of 5% HMW having 1,550,000 M_{w} and 29% MMW having 70,000 Mw; and a second mixture having 5.6% of first HMW having 1,550,000 Mw and 5.6% of second HMW having 1,050,000 Mw and 13.8% MMW having 70,000 Mw.

In some embodiments, the first PIB mixture of MMW and HMW and the second PIB mixture of MMW and HMW are in the ratio of from about 15:1, 14:1, 13:1, 12:1, 11:1, 10:1, 9:1, 8:1, 7:1, 6:1, 5:1, 4:1, 3:1, 2:1, to about 1:1. In some embodiments, PIB comprises the first mixture from about 5, 10, 15, 20, 25, 30, 35, 40, 45, 50, 55, 60, 65, 70, 75, 80, 85, 90, 95% of total PIB and the second mixture from about 95, 90, 85, 80, 75, 70, 65, 60, 55, 50, 45, 40,35, 30, 25, 20, 15, 10, to about 5% of total PIB. In some embodiments, the first PIB mixture comprises 90% of total PIB and the second PIB mixture comprises 10% of total PIB.

In some embodiments, the PIB has a weight from about 110, 111, 112, 113, 114, 115, 116, 117,118, 119, 120, 121, 122, 123, 124, 125, 126, 127, 128, 129, 130, 131 to about 132 mg per 14 cm² of the matrix. In some embodiments the PIB comprises the first PIB mixture having a weight from about 100, 101, 102, 103, 105, 106, 107, 108, 109, 110, 111, 112, 113, 114, 115, 116, 117, to about 118 mg per 14 cm² and the second PIB mixture has a weight from about 10, 11, 12, 13 to about 14 mg per 14 cm². In one embodiment, the first PIB mixture has a weight of 112.4 mg per 14 cm² and the second PIB mixture has a weight of 12.4 mg per 14 cm².

In some embodiments, a suitable material for a PIB first component is PIB X having a solids percentage between 23.5 to 26.5. In some embodiments, the solids percentage is from about 21, 21.5, 22, 22.5, 23, 23.5, 24, 24.5, 25, 25.5, 26, 26.5, 27, 27.5, 28, 28.5 to about 29 percent. In some embodiments, PIB X also comprises an Oppanol® mixture of N100/N80. In some embodiments, PIB X also comprises an Oppanol® mixture N100/N80/B12.

In some embodiments, a suitable material for a PIB second component is PIB Y having a solid percentage between 35-36. In some embodiments, the solid percentage is from about 32, 32.5, 33, 33.5, 34, 34.5, 35, 35.5, 36, 36.5, 37, 37.5, 38, 38.5, 39 to about 39.5 percent. In some embodiments, PIB Y also comprises an Oppanol® mixture of N100/B10.

In some embodiments, the PIB mixture further comprises heptane or n-heptane from about 50% to about 90%. In some embodiments, heptane or n-heptane is from about 50, 51, 52, 53, 54, 55, 56, 57, 58. 59. 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, to about 90%. In some embodiments, the PIB mixture comprises about 61.9% heptane.

In some embodiments, PIB Y comprises an Oppanol® mixture of 5% N100, 29% of B10 and 66% of n-heptane by weight. In other embodiments, the PIB X comprises 5.6% of Oppanol® N100, 5.6% of N80, 13.8% of B12SFN, and 25% of n-heptane by weight.

In some embodiments, N100 comprises a solid percentage from about 23 to about 31 percentage. In some embodiments, the solid percentage is from about 23, 23.5, 24, 24.5, 25, 25.5, 26, 26.5, 27, 27.5, 28, 28.5, 29, 29.5, 30, 30.5, to about 31 percent. N100 has a viscosity from about 25000 to about 60000 cP. In some embodiments, the viscosity is from about 25000, 25500, 26000, 26500, 27000, 27500, 28000, 28500, 29000, 29500, 30000, 30500, 31000, 31500, 32000, 32500, 33000, 33500, 34000, 34500, 35000, 35500, 36000, 36500, 37000, 37500, 38000, 38500, 39000, 40000, 41000, 42000, 43000, 44000, 45000, 50000, 55000 to about 60000 cP. N100 has a peel from about 22 to about 30 oz/in. In some embodiments, the peel is from about 22, 25, 30, 35, 40, 45, 50, to about 55 ounce per inch. N100 has a shear from about 37 to about 46 2.2 psi·h. In some embodiments, the shear is from about 36, 36.5, 37, 38, 38.5, 39, 40, 40.5, 41, 41.5, 42, 42.5, 43, 43.5, 44, 44.5, 45, 45.5, to about 46. In one embodiment, N100 has about 25.8% solid percentage, viscosity of about 47200 cP, peel of about 27 oz/in, and shear of about 42.2 2.2 psi·h. In another embodiment, the N100 has about 26% solid percentage, viscosity of about 41600 cP, peel of about 24.5 cP, and shear of about 41.21 22 psi·h. In some embodiments, Oppanol® N100 has a viscosity average molecular weight Mᵥ of 1,110,000, a weight average molecular weight M_{W} of 1,550,000, and a molecular weight distribution (Mw/Mn) of 2.9.

In some embodiments, N80 comprises a solid percentage from about 29 to about 34 percentage. In some embodiments, the solid percentage is from about 29, 29.5, 30, 30.5, 31, 31.5, 32, 32.5, 33, 33.5, to about 34 percent. N80 can have a viscosity from about 20000 to about 60000 cP. In some embodiments, the viscosity is from about 20000, 21000, 22000, 23000, 24000, 25000, 25500, 26000, 26500, 27000, 27500, 28000, 28500, 29000, 29500, 30000, 30500, 31000, 31500, 32000, 32500, 33000, 33500, 34000, 34500, 35000, 35500, 36000, 36500, 37000, 37500, 38000, 38500, 39000, 40000, 41000, 42000, 43000, 44000, 45000, 50000, 55000, to about 60000 cP. N80 has a peel from about 21 to about 38 oz/in. In some embodiments, the peel is from about 20, 21, 22, 23, 24, 25, 26, 27, 28, 29, 30, 31, 32, 33, 34, 35, 36, 37, 38, 39, to about 40 ounce per inch. N80 has a shear from about 12 to about 17 2.2 psi·h. In some embodiments, the shear is from about 12, 12.5, 13, 13.5, 14, 14.5, 15, 15.5, 16, 16.5, to about 17 2.2 psi·h. In one embodiment, N80 has about 34.3% solid percentage, viscosity of about 43300 cP, peel of about 23.1 oz/in, and shear of about 13.7 2.2 psi·h. In another embodiment, the N80 has about 33.7% solid percentage, viscosity of about 40950 cP, peel of about 35.1 cP, and shear of about 15.5 22 psi·h. In some embodiments, Oppanol® N80 has a viscosity average molecular weight Mᵥ of 800,000, a weight average molecular weight M_{W} of 1,050,000, and a molecular weight distribution (Mw/Mn) of 2.4.

In some embodiments, Oppanol® B12SFN has a viscosity average molecular weight Mᵥ of 55,000, a weight average molecular weight M_{w} of 70,000, and a molecular weight distribution (Mw/Mn) of 3.2. Oppanol® B10SFN has a viscosity average molecular weight Mᵥ of 40,000, a weight average molecular weight M_{w} of 53,000, and a molecular weight distribution (Mw/Mn) of 3.2.

In some embodiments, the PIB mixture further comprises water, solvent blend such as n-heptane, heptane, hexane, ethyl acetate, toluene, alcohol including ethanol, isopropyl alcohol, methanol, butanol, propanol, pentanol or a combination thereof. In some embodiments, the PIB mixture comprises water from about 0.005, 0.0055, 0.006, 0.0062, 0.0065, 0.007, 0.0075, 0.008, 0.0085, 0.009, 0.0095 0.01 0.0156, 0.0248, 0.03, 0.04, 0.05, 0.051 0.055 to about 0.06 percent. In some embodiments, the PIB mixture comprises solvent blend from about 98, 99, 99.1, 99.2, 99.3, 99.4, 99.5, 99.6, 99.7, 99.8 to about 99.9 percent. In some embodiments, the PIB mixture comprises hexane from 0% to about 0.5%. In some embodiments, the PIB mixture comprises ethyl acetate from 0% to about 0.5%. In some embodiments, the PIB mixture comprises toluene from 0% to about 0.5%. In one embodiment, N100 comprises 0.02 ethyl acetate and 0.15 toluene. In one embodiment, N80 comprises 0.02 ethyl acetate and 0.02 toluene.

The above measurements can be measured in any known methods conducted by one of ordinary skill in the art. For example, the solid percentage can be calculated through bulk density, statistical approach and many other methods. Viscosity can be measured in the Brookfield viscometer. Peel can be measured through peel strength test involving various peeling angles and speed. Shear can be measured through a polymer shear test, which measures the polymer's ability to withstand forces that can cause the internal structure to slide against itself.

The silicone adhesives that may be used in forming the matrix are typically high molecular weight polydimethyl siloxanes or polydimethyldiphenyl siloxanes. Formulations of silicone adhesives that are useful in transdermal patches are described in U.S. Pat. Nos. 5,232,702, 4,906,169 and 4,951,622. In various aspects, the adhesive comprises, consists essentially of, or consists of a solution of polyacrylate, a silicone or polyisobutylene.

The PIB adhesive may also comprise a tackifier such as polybutene oil, a plasticizer such as mineral oil, or a high Tg, medium molecular weight aliphatic resins such as the ESCOREZ™ resins available from Exxon Chemical.

In various embodiments, the adhesive is in the matrix in an amount of about 58% w/w to about 58.05% w/w based on a total % w/w of the matrix. In other embodiments, the transdermal system is in a patch or film form and the adhesive is in the matrix in an amount of about 121.8 mg to about 121.9 mg per patch or film. In one aspect, the adhesive is in the matrix in an amount of about 58.07% w/w based on a total % w/w of the matrix. In another aspect, the adhesive is in the matrix in an amount of about 87.11 wt. g/m² based on a total wt. g/m² of the matrix.

In addition to the pressure sensitive adhesive, norelgestromin, and optional estrogen, the matrix will typically contain sufficient amounts of permeation enhancers to increase the permeability of the norelgestromin and estrogen through the skin and provide the desirable amount of active pharmaceutical ingredients. Examples of skin permeation enhancers that may be included in the matrix are described in U.S. Pat. Nos. 5,059,426; 4,973,468; 4,906,463; and 4,906,169, and include, but are not limited to lactate ester of C12 to C18 aliphatic alcohol, lauryl lactate, oleic acid, oleyl alcohol, or propylene glycol monolaurate (PGML). The amount of permeation enhancer included in the matrix will depend upon the particular enhancer(s) used. In most instances, the enhancer will constitute in the range of 0.5 to 20% by weight of the matrix.

Other permeation enhancers include, but are not limited to, polyhydric alcohols such as dipropylene glycol, propylene glycol, and polyethylene glycol; oils such as olive oil, mineral oil, light mineral oil, squalene, and lanolin; fatty ethers such as cetyl ether and oleyl ether; fatty acid esters such as isopropyl myristate; urea and urea derivatives such as allantoin which affect the ability of keratin to retain moisture; polar solvents such as dimethyidecylphosphoxide, methyloctylsulfoxide, dimethyllaurylamide, dodecylpyrrolidone, isosorbitol, dimethylacetonide, dimethylsulfoxide, decylmethylsulfoxide, and dimethylformamide which affect keratin permeability; salicylic acid which softens the keratin; amino acids which are penetration assistants; benzyl nicotinate which is a hair follicle opener; and higher molecular weight aliphatic surfactants such as lauryl sulfate salts which change the surface state of the skin and drugs administered. Other agents include oleic and linoleic acids, ascorbic acid, panthenol, butylated hydroxytoluene, tocopherol, tocopheryl acetate, tocopheryl linoleate, propyl oleate, and isopropyl palmitate.

The amount of permeation enhancer included in the matrix will depend upon the particular enhancer(s) used. In most instances the enhancer will be in the range of 0.5 to 20% by weight of the matrix.

In one embodiment, the permeation enhancer is oleyl alcohol. In some embodiments, oleyl alcohol comprises from about 3.2, 3.3, 3.4, 3.5, 3.6, 3.7, to about 3.8 percent by weight of the matrix. In one embodiment, oleyl alcohol comprises about 23.19% by weight. In some embodiments, oleyl alcohol comprises about 6.7, 6.8, 6.9, 7.0, 7.1, 7.2, 7.3, 7.4, 7.5, 7.6, 7.7, 7.8, 7.9, 8.0, to about 8.1 mg per 14 cm². In one embodiment, oleyl alcohol comprises about 7.65 per 14 cm². In another embodiment, the penetration enhancer is a glycol, such as dipropylene glycol, propylene glycol, butylene glycol or polyethylene glycol. In other embodiments, the penetration enhancer comprises a mixture of at least two penetration enhancers. In some embodiments, the enhancer comprises dipropylene glycol LO+. In some embodiments, dipropylene glycol LO+ comprises from about 0.68, 0.69, 0.70, 0.71, 0.72, 0.73, 0.74, 0.75, 0.76, 0.77, 0.78, 0.79, 0.80, 0.81, to about 0.82 percent by weight of the matrix. In one embodiment, dipropylene glycol LO+ comprises about 0.77% by weight of the matrix. In some embodiments, dipropylene glycol LO+ comprises from about 1.5, 1.6, 1.7, to about 1.8 mg per 14 cm². In one embodiments, dipropylene glycol LO+ comprises about 1.61 mg per 14 cm².

In various embodiments, the matrix may contain other additives depending upon the particular adhesive used. For instance, materials, such as polyvinyl pyrrolidone (PVP), that inhibit drug crystallization, hygroscopic agents that improve the duration of wear, or additives that improve the physical (e.g., cold flow) or adhesive (e.g., tack, cohesive strength) properties of the matrix may be included.

A crystallization inhibitor or solubility enhancer may also be employed in the current application, for example polyvinylpyrrolidone polymers, polyethylene oxide, polyacrylic acid, polyvinyl alcohol, silicone dioxide, silica, celluloses and cellulose derivatives such as hydroxymethyl cellulose, hydroxypropyl cellulose, gelatins, gums, starches, dextrins and dextrans, sterols, bile acids and other absorptive agents that possess the capability to absorb and hold water or moisture.

Particularly preferred compounds are PVPs. The term “polyvinylpyrrolidone” or “PVP” refers to a polymer, either a homopolymer or copolymer, containing vinylpyrrolidone (also referred to as N-vinylpyrrolidone, N-vinyl-2-pyrrolidone and N-vinyl-2-pyrrolidinone) as a monomeric unit. PVP polymers include soluble and insoluble homopolymeric PVPs, and copolymers such as vinylpyrrolidone/vinyl acetate and vinylpyrrolidone/dimethylamino-ethylmethacrylate. The cross-linked homopolymer is insoluble and is generally known in the pharmaceutical industry under the designations polyvinylpolypyrrolidone, crospovidone and PVP.

PVPs are sold to the pharmaceutical industry under the trademarks KOLLIDON® by BASF (Parsippany, N.J.); PLASDONE™, POLYPLASDONE™ and COPOLYMER 958 by ISP Technologies (Wayne, N.J.) Other PVPs are KOLLIDON® CL-F, KOLLIDON® CL-SF, and KOLLIDON® CL-M.

Typically, the PVP is present in an amount from about 5% to about 50% by weight, preferably from about 10% to about 40% by weight based on the dry weight of the total adhesive matrix composition. However, the amount of PVP can be higher than 20% for example, up to 40%, depending on the particular drug used and on the desired properties of the matrix blend. In some embodiments, PVP comprises crospovidone from about 18, 19, 20, 21, 22, 23, 24, to about 25% by weight. In some embodiments, crospovidone is about 23.19% by weight. In some embodiments, crospovidone comprises from about 42, 43, 44, 45, 46, 47, 48, 49, 50, 51, to about 52 mg per 14 cm² of the matrix. In one embodiment, crospovidone is about 48.7 mg per 14 cm².

The release rate or delivery rate of the active from the transdermal system, onset of delivery (lag time) and delivery profile of the drug may be selectively modulated by one or more of (a) increasing or decreasing the thickness or coat weight of the acrylic-based adhesive coating per cm² as applied to the backing or fabric of the drug release layer of the system, (b) manipulating the moiety or functionality of the acrylic-based adhesive coating, and (c) manipulating the monomeric composition and/or ratios of the acrylic-based adhesive coating. Either the non-drug containing coating or the carrier composition must also be a pressure-sensitive adhesive when used as area of attachment to the skin or mucosa of the user. The drug carrier composition may be comprised of (a) one or more acrylic-based polymers having one or more functionality alone or in combination with (b) one or more silicone-based polymers having one or more silanol contents (capping) and/or resin to polymer ratios, and are present in proportions to provide a desired solubility for the drug. Further manipulation of drug delivery, onset and profiles can be achieved by varying the concentrations of the drug in the drug-loaded carrier.

Now referring to FIG. 15, it is a schematic illustration of a representative transdermal system. The outermost backing can be a polyethylene/polyester film. The middle layer can be a polyisobutylene adhesive matrix as discussed above containing, for example, the two active pharmaceutical ingredients, for example, norelgestromin and ethinyl estradiol. The matrix also can contain several inactive ingredients, for example, oleyl alcohol, dipropylene glycol LO+, crospovidone, nonwoven polyester, and light mineral oil. In some embodiments, the light mineral oil in the matrix comprises from about 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, to about 14% by weight. In some embodiments, light mineral oil is about 12.89% by weight. In some embodiments, light mineral oil comprises from about 24, 25, 26, 27, to about 28 mg per 14 cm² of the matrix. In one embodiment, light mineral oil is about 27.07 mg per 14 cm². The third layer can be a release liner that is slit near the middle to facilitate removal prior to use. This release liner can be a transparent, fluoropolymer coated polyester film, and both pieces are removed from the patch and discarded prior to use.

One embodiment of the transdermal delivery system comprises a 14 cm² or less transdermal system, optionally with rounded corners, comprising or consisting of a backing film, an adhesive layer containing nonwoven fabric, and a clear oversized removable release liner, and further comprising an API (e.g., norelgestromin and/or ethinyl estradiol). Each individual transdermal system is placed between two pieces of protective film and packaged in a sealed pouch, which is imprinted with lot number and manufacturing date.

Each transdermal system can contain one or more active pharmaceutical ingredients or contraceptive agents, which comprise, consists essentially of, or consists of norelgestromin (NGMN) and/or ethinyl estradiol (EE) that are dissolved in a pressure-sensitive adhesive matrix and the matrix is designed to deliver norelgestromin and ethinyl estradiol transdermally. In some embodiments, the transdermal delivery system of this application contains three layers, wherein the matrix comprises norelgestromin 4.86 mg and ethinyl estradiol 0.264 mg per 14 cm² of the matrix. In some embodiments, ethinyl estradiol comprises about 0.53 mg per 14 cm² of the matrix. In some embodiments, the transdermal system can release the contraceptive agent from the transdermal system in an amount about 100 mcg per day to about 300 per day. In some embodiments, the transdermal system is configured to release norelgestromin in an amount from about 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, to about 272 mcg per day. In some embodiments, the transdermal system is configured to release norelgestromin in an amount from 214 mcg per day to 239 per day. In some embodiments, the transdermal system is configured to release norelgestromin in an amount from about 187 mcg per day to about 197 mcg per day. In some embodiments, the transdermal system is configured to release norelgestromin in an amount from about 193 mcg per day to about 196 mcg per day. In some embodiments, the transdermal system is configured to release norelgestromin in an amount from about 184 mcg per day to about 187 mcg per day. In some embodiments, the transdermal system is configured to release norelgestromin in an amount from about 184 mcg per day to about 200 mcg per day. In some embodiments, the transdermal system is configured to release norelgestromin in an amount from about 120 mcg per day to about 230 mcg per day. In some embodiments, the transdermal system is configured to release norelgestromin in an amount from about 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, to about 132 mcg per day. In some embodiments, the transdermal system is configured to release norelgestromin in an amount from about 214 to about 248 mcg per day. In some embodiments, the transdermal system is configured to release norelgestromin in an amount from about 103 mcg per day to about 129 mcg per day. In some embodiments, the transdermal system is configured to release norelgestromin in an amount from about 195 mcg per day to about 202 mcg per day. In some embodiments, the transdermal system is configured to release norelgestromin in an amount from about 185 mcg per day to about 202 mcg per day.

In some embodiments, the matrix of the transdermal system contains ethinyl estradiol in an amount of about 0.125% w/w to about 0.19% w/w based on a total % w/w of the matrix. In other aspects, the transdermal system is in a patch or film form and contains ethinyl estradiol in the matrix in an amount of about 0.264 mg to about 0.396 mg per patch or film. In the matrix, in some aspects, the ethinyl estradiol can be in an amount of about 0.13% w/w based on a total % w/w of the matrix. In other aspects, the amount of ethinyl estradiol in the matrix is in an amount of about 0.189 wt. g/m² based on a total wt. g/m² of the matrix. In some embodiments, the transdermal system can release the contraceptive agent from the transdermal system in an amount about 100 mcg per day to about 300 per day. In some embodiments, the transdermal system is configured to release ethinyl estradiol in an amount from about 14, 15, 16, 17, 18, 19, 20, 21, 22, 23, 24, 25, 26, 27, 28, 29, 30 to about 31 mcg per day. In some embodiments, the transdermal system is configured to release ethinyl estradiol in an amount from about 19 mcg per day to about 23 mcg per day. In some embodiments, the transdermal system is configured to release ethinyl estradiol in an amount from about 17 mcg per day to about 18 mcg per day. In some embodiments, the transdermal system is configured to release ethinyl estradiol in an amount from about 15 mcg per day to about 29 mcg per day. In some embodiments, the transdermal system is configured to release ethinyl estradiol in an amount from about 16 mcg per day to about 28 mcg per day.

The coat weight of the matrix can range from 100-200 g/m². In some embodiments, the coat weight of the matrix may be 125-175 g/m², 140-160 g/m², or 145-155 g/m². In one embodiment, the coat weight of the matrix is about 150 g/m². In one embodiment, the coat weight of the matrix may be 7 to 15 mg/cm². Table 1 includes the coat weight of 150 g/m² of the matrix, which includes one or more active pharmaceutical agents, crospovidone, dipropylene glycol lo+, oleyl alcohol, and/or mineral oil.

The transdermal system of this application, in one embodiment, has constant contraceptive agent release at about 48 hours to about 168 hours after the transdermal system is applied to the skin of the patient. In some embodiments, as illustrated in the examples of this application, the transdermal system is configured to be worn 7 days and release the active ingredients over a period of seven days.

The Pearl Index is a well-established and widely accepted quantitative measure of contraceptive effectiveness and has been routinely used in clinical research and regulatory evaluation of hormonal contraceptives. The Pearl Index represents the number of unintended pregnancies observed per 100 women who use a particular contraceptive method for one year (i.e., 100 woman-years of exposure). A lower Pearl Index corresponds to greater contraceptive efficacy, reflecting fewer observed pregnancies among users over time. The Pearl Index is calculated by dividing the number of confirmed on-treatment pregnancies by the total duration of exposure, expressed either as total months of use and multiplied by 1,200 or as total cycles of use and multiplied by 1,300. By way of example, a Pearl Index of 1 indicates that one pregnancy occurred among 100 women using the method continuously for one year. Accordingly, incremental changes in Pearl Index are clinically meaningful and are commonly used to compare efficacy across formulations, dosing regimens, and patient subpopulations, including body mass index cohorts. In general, reductions in estrogen doses would conventionally be expected to decrease contraceptive effectiveness and to be associated with an increase in Pearl Index, absent other compensatory formulation or pharmacokinetic factors.

In some embodiments, the transdermal system has a Pearl Index of between 1.0 and less than 6.0. In some embodiments, the transdermal system has a Pearl Index from about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, to about 6.0. In one embodiment, the Pearl Index is between 1.0 to less than 5.0. In another embodiment, the Pearl Index is between 1.0 to less than 5.0. In one embodiment, the transdermal system has a Pearl Index of between 1.5 to less than 4.14. In another embodiment, the transdermal system has a Pearl Index of between 2.7 to less than 5.95. In one embodiment, the transdermal system has a Pearl Index of 4.14. In another embodiment, the transdermal system has a Pearl Index of 4.18.

In some embodiments, the Pearl Index for BMI of less than 25 is from about 4.00, 4.05, 4.10, 4.15, 4.20, 4.25, 4.30, 4.35, 4.40, 4.45, 4.48, 4.50, 4.55, 4.60, 4.65, 4.70, 4.75, 4.80, 4.85, 4.90, 4.95, 5.00. In one embodiment, the Pearl Index for BMI of less than 25 is about 4.48. In some embodiments, the Pearl Index for BMI of greater than or equal to 25 and less than 30 is from about 3.00, 3.05, 3.10, 3.15, 3.20, 3.25, 3.30, 3.35, 3.40, 3.45, 3.50, 3.55, 3.60, 3.65, 3.70, 3.75, 3.76, 3.80, 3.85, 3.90, 3.95, to about 4.00. In one embodiment, the Pearl Index for BMI of greater than or equal to 25 and less than 30 is about 3.76. In some embodiments, the Pearl Index for BMI of greater or equal to 30 is from about 4.00, 4.05, 4.10, 4.15, 4.20, 4.25, 4.30, 4.35, 4.40, 4.45, 4.50, 4.55, 4.60, 4.65, 4.70, 4.75, 4.80, 4.85, 4.86, 4.90, 4.95, 5.00, 5.05, 5.10, 5.15, 5.20, 5.25, 5.30, 5.35, 5.40, 5.45, 5.50, 5.55, 5.60, 5.65, 5.70, 5.75, 5.80, 5.85, 5.90, 5.95, 6.00. In one embodiment, the Pearl Index for BMI of greater than 30 is about 3.86. In some embodiments, the Pearl Index for BMI has an overall vale for BMI less than 25, BMI greater than or equal to 25 and less than 30, BMI greater than 30, from about 4.00, 4.05, 4.10, 4.14, 4.15, 4.20, 4.25, 4.30, 4.35, 4.40, 4.45, 4.50, 4.55, 4.60, 4.65, 4.70, 4.75, 4.80, 4.85, 4.90, 4.95, 5.00, 5.05, 5.10, 5.15, 5.20, 5.25, 5.30, 5.35, 5.40, 5.45, 5.50, 5.55, 5.60, 5.65, 5.70, 5.75, 5.80, 5.85, 5.90, 5.95, 6.00. In one embodiment, the Pearl Index for overall BMI cohorts is about 4.14.

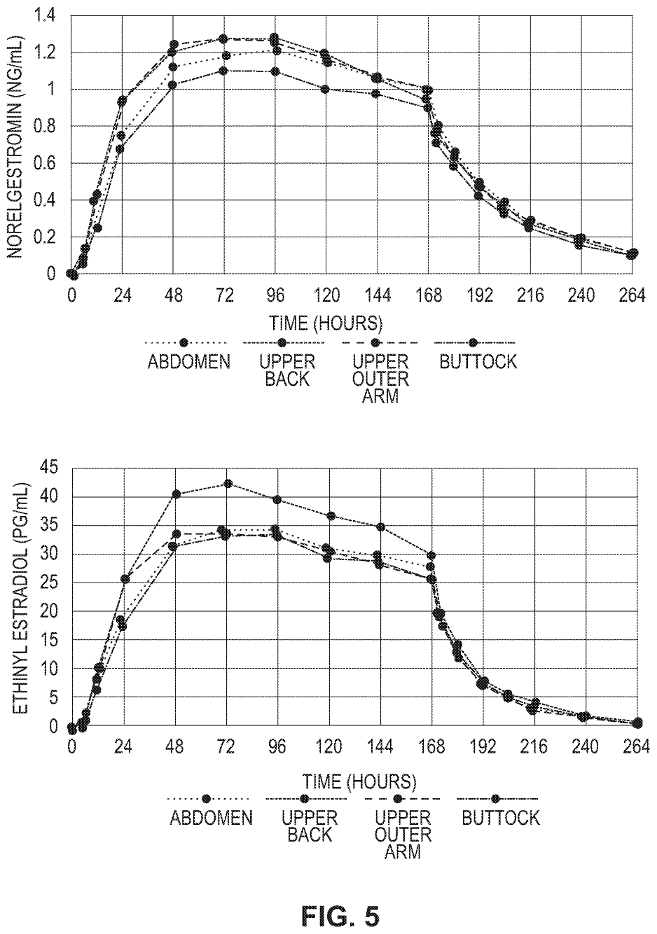

FIG. 5 illustrates a graph of norelgestromin 4.86 mg and ethinyl estradiol 0.264 mg in a transdermal system following a 7-day wear period on the abdomen, upper back, upper outer arm, and buttock in healthy adult female volunteers. The transdermal system has the matrix with a combination of two different PIB mixtures. In this embodiment, the patch applied having norelgestromin arrives at the highest peak at 1.369 ng/mL on the upper outer arm and the ethinyl estradiol arrives at the highest peak at 38.25 pg/mL at the upper outer arm.

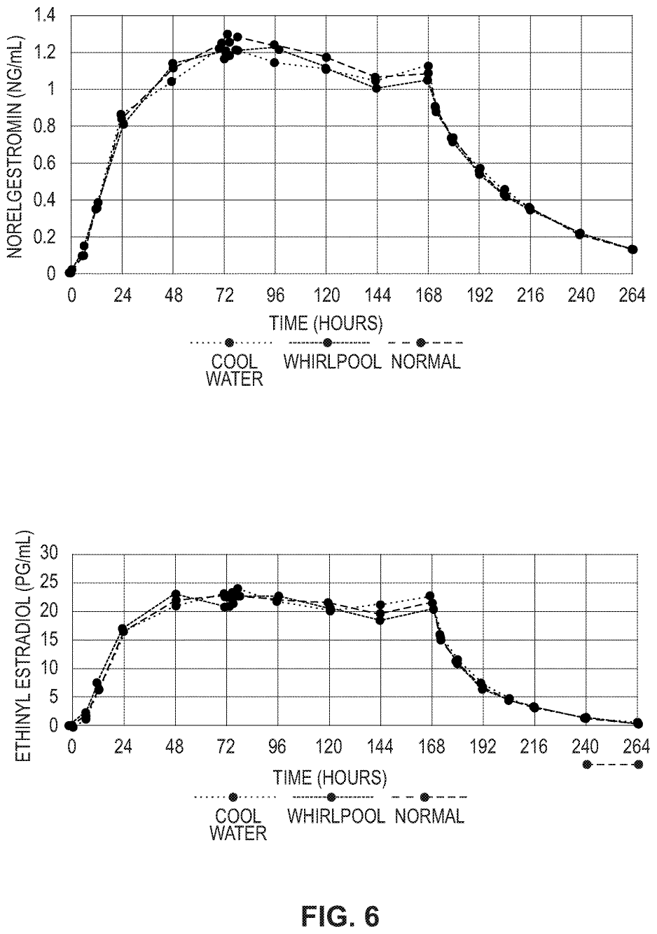

FIG. 6 illustrates a graph of norelgestromin 4.86 mg and ethinyl estradiol 0.264 mg in a transdermal system following a 7-day wear period on the abdomen in healthy adult female volunteers under normal, cool water, and whirlpool conditions. In this embodiment, the patch applied to the abdomen site has norelgestromin and has the highest peak at 1.439 ng/mL under a normal wear condition and the ethinyl estradiol has the highest peak at 30.035 pg/mL under cool water condition. In some embodiments, the cool water temperature is from about 20 to about 22 degrees Celsius and the whirlpool is at a temperature from about 39 to about 41 degrees Celsius.

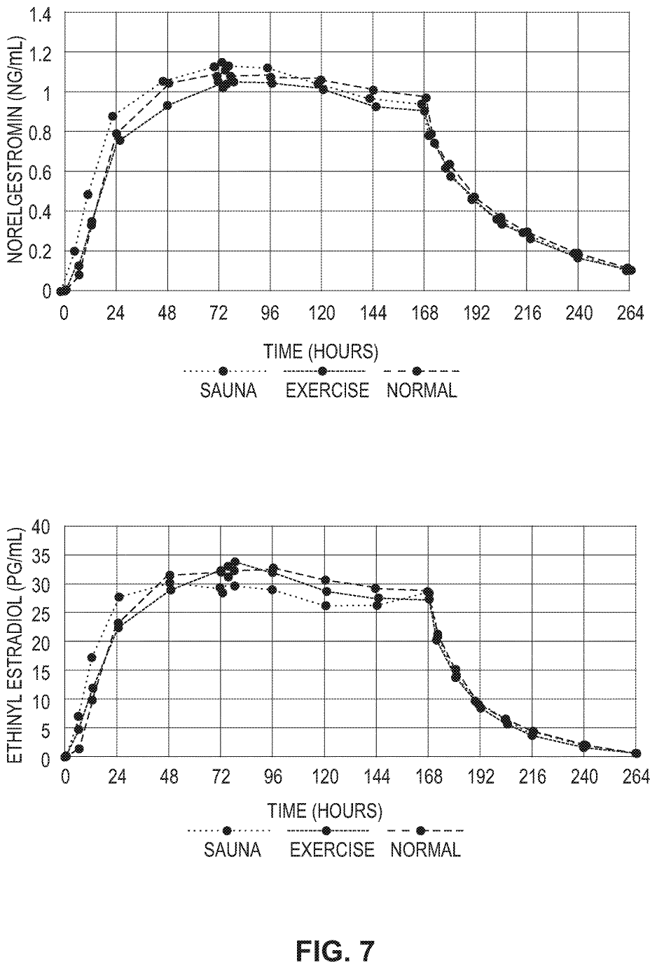

FIG. 7 illustrates a graph of norelgestromin 4.86 mg and ethinyl estradiol 0.264 mg in a transdermal system following a 7-day wear period on the abdomen in healthy adult female volunteers under normal, sauna and exercise conditions. In this embodiment, the patch is applied to the abdomen site and has norelgestromin in it and has the highest peak at 1.303 ng/mL under a sauna and the ethinyl estradiol has the highest peak at 42.377 pg/mL under normal condition.

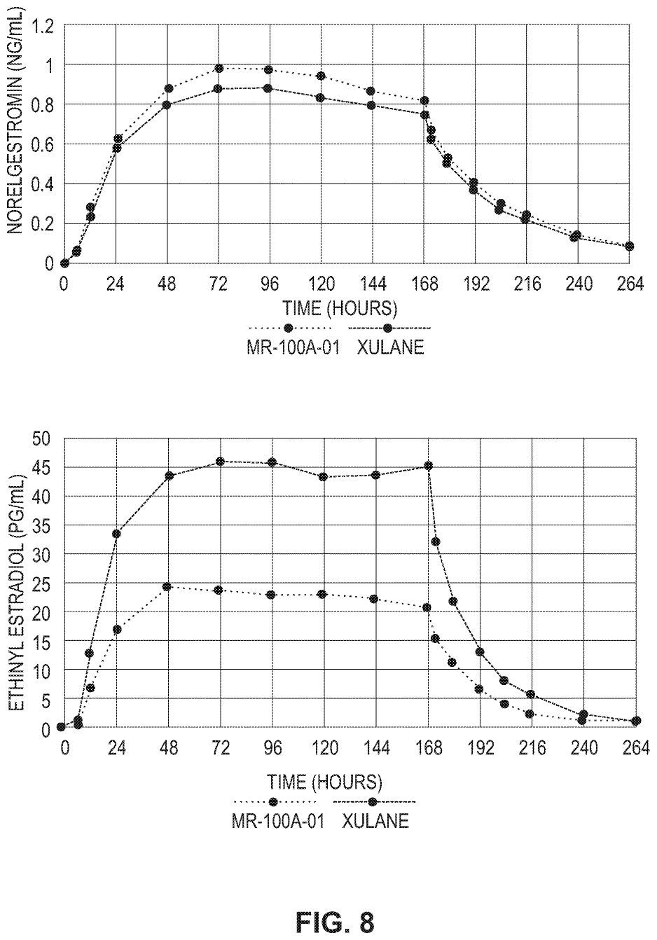

FIG. 8 illustrates a graph of a single-dose comparative bioavailability and adhesion study of norelgestromin 4.86 mg/ethinyl estradiol 0.264 mg in a transdermal system (14 cm²)(Unit E) and a commercially marketed product, XULANE® Transdermal System (norelgestromin, 4.86 mg and ethinyl estradiol, 0.53 mg/14 cm²) following a 7-day wear period on the lower abdomen in healthy adult female volunteers. In this embodiment, the patch applied to the abdomen site having norelgestromin has the highest peak at 1.02694 ng/mL compared to the commercially marketed product XULANE®, which has the concentration peak at 0.9451 ng/mL and the ethinyl estradiol from the matrix has the highest peak of 38.393 pg/mL compared to XULANE®'s 54.690 pg/mL. Unit E delivered a higher concentration of norelgestromin over time at about 166 ng*hr/mL, which is 10% higher than the XULANE® Transdermal System.

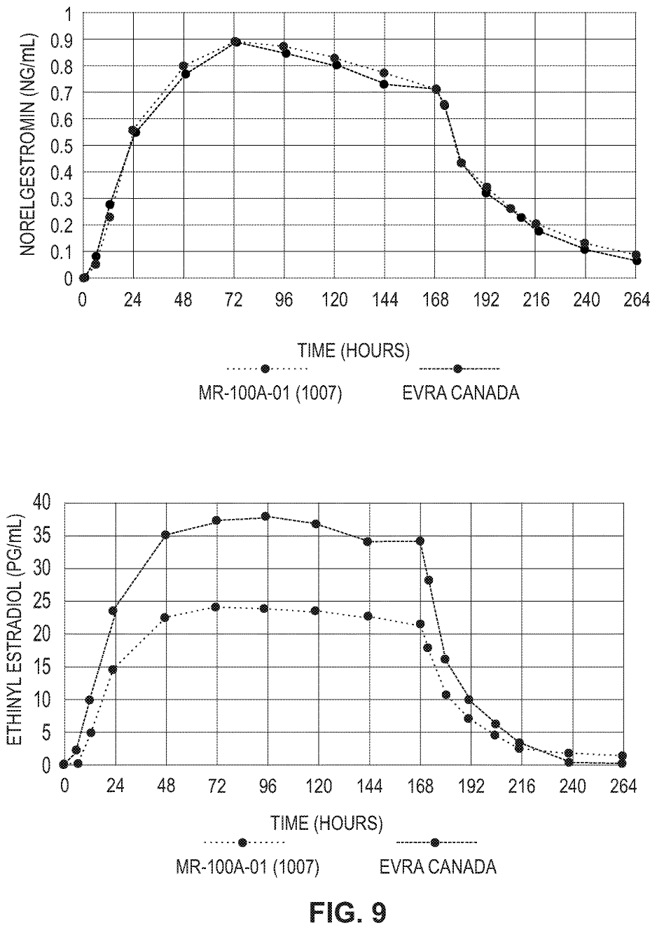

FIG. 9 illustrates a graph of a single-dose comparative bioavailability and adhesion study of norelgestromin 4.86 mg/ethinyl estradiol 0.264 mg in a transdermal system (14 cm²) and, a commercially marketed product, EVRA® Canada following a 7-day wear period on the lower abdomen in healthy adult female volunteers. In this embodiment, the patch applied to the abdomen site having norelgestromin has the highest peak at 0.927 ng/mL compared to the commercially marketed product EVRA® Canada, which has the concentration peak at 0.903 ng/mL and the ethinyl estradiol from the matrix has the highest peak at 27.89 pg/mL compared to EVRA® Canada 41.177 pg/mL.

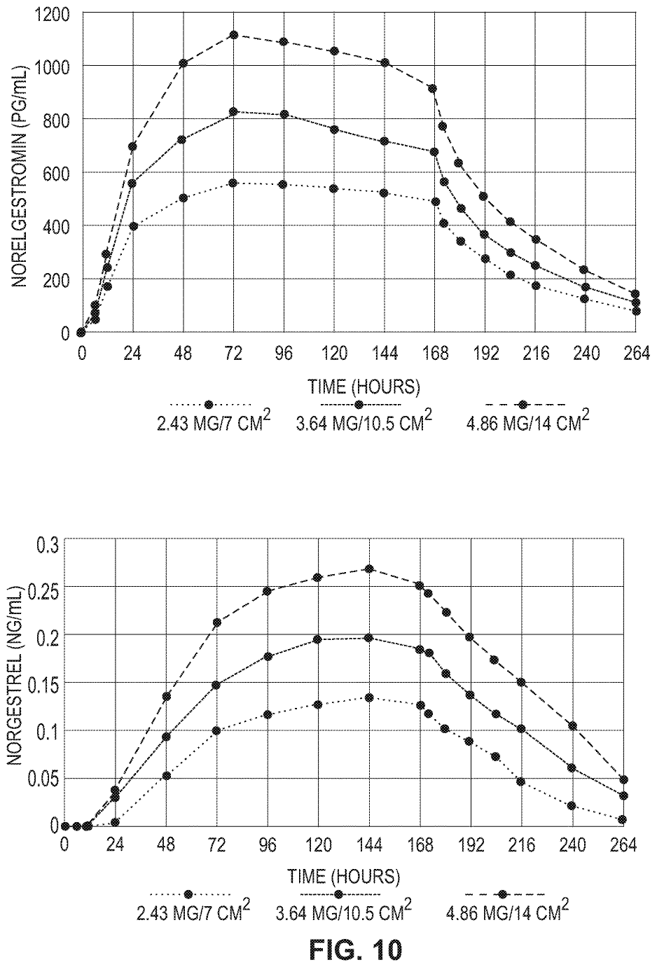

FIG. 10 illustrates a graph of a single-dose bioavailability and adhesion study comparing three different patch sizes of norelgestromin transdermal systems following a 7-day wear period in healthy adult female volunteers. In some embodiments, the patch has 2.43 mg norelgestromin/7 cm² matrix and reaches a concentration peak at 604.255 pg/mL. In some embodiments, the patch has 3.64 mg norelgestromin/10.5 cm² matrix and has a concentration peak at 868.877 pg/mL. In some embodiments, the patch has 4.86 mg norelgestromin/14 cm² matrix and has a concentration peak at 1183.078 pg/mL.

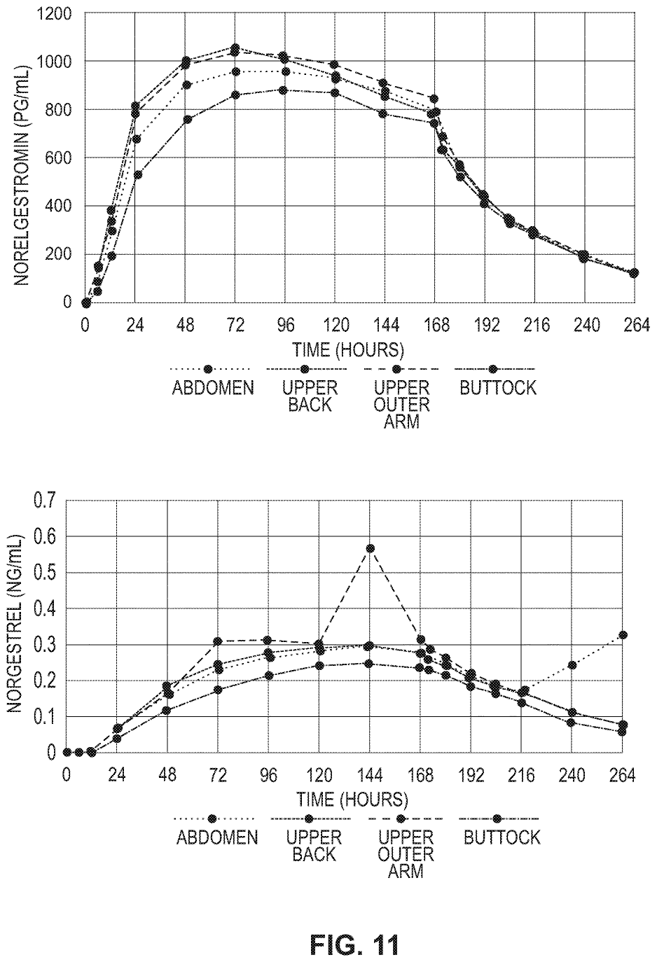

FIG. 11 illustrates a graph illustrating the single-dose pharmacokinetics and adhesive properties of a norelgestromin transdermal system, 4.86 mg/14 cm² following a single application on each of the proposed application sites, which included the lower abdomen, upper back, upper outer arm, and buttock for 7 days under normal conditions. The delivery utilizes the transdermal system having the matrix, which has a unique polyisobutylene (PIB) combination of Medium Molecular Weight (MMW) PIB and High Molecular Weight (HMW) PIB (see PIB X in Table 1). For example, in Table 1, PIB X is a PIB comprising a first component, which is both a medium molecular weight polyisobutylene, for example, having an average molecular weight of about 70,000, which can be obtained from BASF, Florham Park, New Jersey, in the product Oppanol® B-B12 (12N) and a high molecular weight polyisobutylene, for example, having an average molecular weight of about 1,050,000, which can be obtained from BASF, Florham Park, New Jersey, in the product Oppanol® N-80. The PIB, in some embodiments, can be stabilized with butylated hydroxytoluene at an average concentration of 500 ppm.

For example, in Table 1, PIB Y is a PIB comprising a second component, which is both a medium molecular weight polyisobutylene, for example, having an average molecular weight of about 53,000, which can be obtained from BASF, Florham Park, New Jersey, in the product Oppanol® B-B10 (ION) and a high molecular weight polyisobutylene, for example, having an average molecular weight of about 1,550,000, which can be obtained from BASF, Florham Park, New Jersey, in the product Oppanol® N-100. The PIB, in some embodiments, can be stabilized with butylated hydroxytoluene at an average concentration of 500 ppm. These two components can be mixed together in the amounts indicated in Table 1 with the other components indicated in Table 1 to form a matrix, which is applied to a polymer fabric, such as for example, non-woven polyester to form the medicated or middle layer of the patch, which is disposed between the backing and the release liner.

In this embodiment shown in FIG. 11, the patch applied having norelgestromin obtains a highest peak of 1090.142 pg/mL on the upper back and the norgestrel metabolite has the highest peak of 0.657 ng/mL. Norgestrel plasma concentrations were also measured to ensure that the norgestrel was being produced proportionally to the concentration of norelgestromin and excess norgestrel was not accumulating over time.

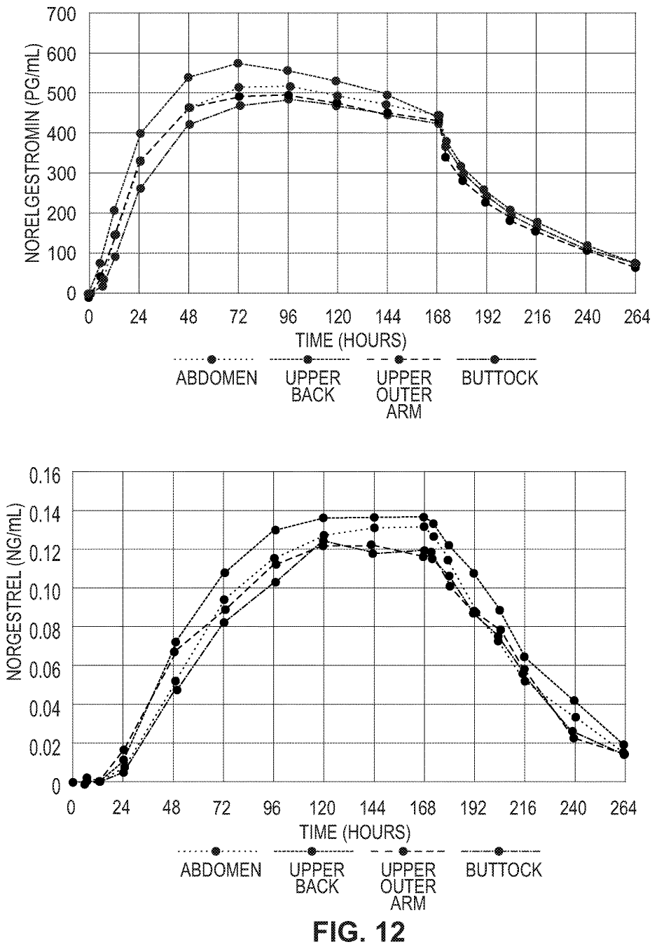

FIG. 12 illustrates a graph of a single-dose comparative bioavailability and adhesion study of norelgestromin transdermal systems, 2.43 mg/7 cm² following a 7-day wear period on the lower abdomen, upper back, upper outer arm, and buttock in healthy adult female volunteers. The delivery utilizes the transdermal system having the matrix with a combination of two different PIB mixtures. In this embodiment, the patch applied having norelgestromin arrives at the highest peak at 601.359 pg/mL on the upper back and the norgestrel metabolite at the highest peak at 0.151 ng/mL.

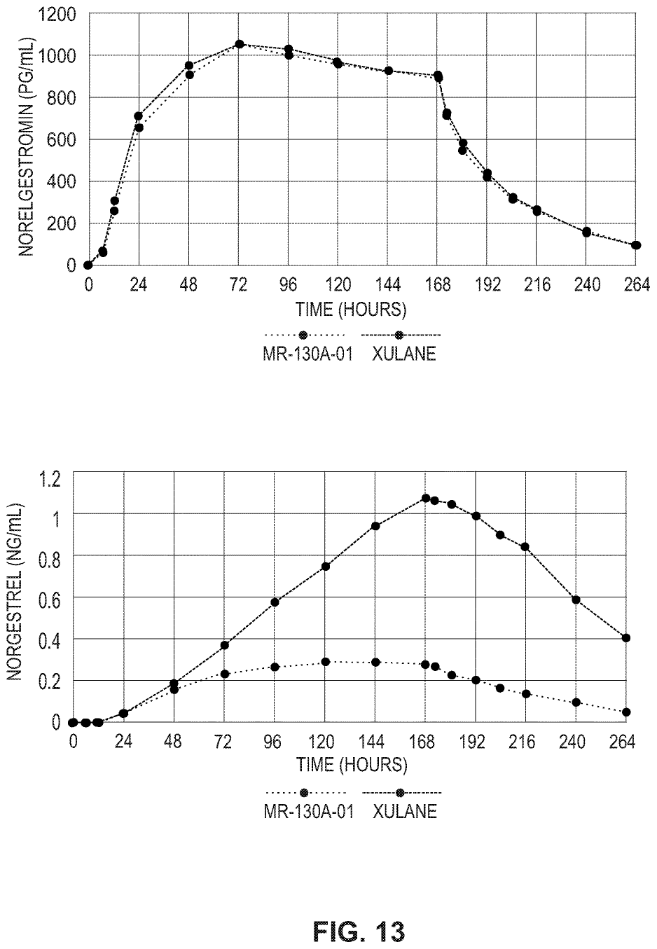

FIG. 13 illustrates a graph of a single-dose comparative bioavailability and adhesion study of norelgestromin transdermal systems, 4.86 mg/14 cm² and XULANE® transdermal system (norelgestromin, 4.86 mg and ethinyl estradiol, 0.53 mg/14 cm²) following a 7-day wear period on the lower abdomen in healthy adult female volunteers. In this embodiment, the patch applied to the abdomen site having norelgestromin has the highest peak at 1107.048 ng/mL compared to the commercially marketed product XULANE®, which has the concentration peak at 1152.242 ng/mL. The norelgestromin-only patch formulation has the highest norgestrel metabolite peak of 0.316 ng/mL compared to commercially available XULANE®, which had its highest norgestrel metabolite peak of 1.107 ng/mL.

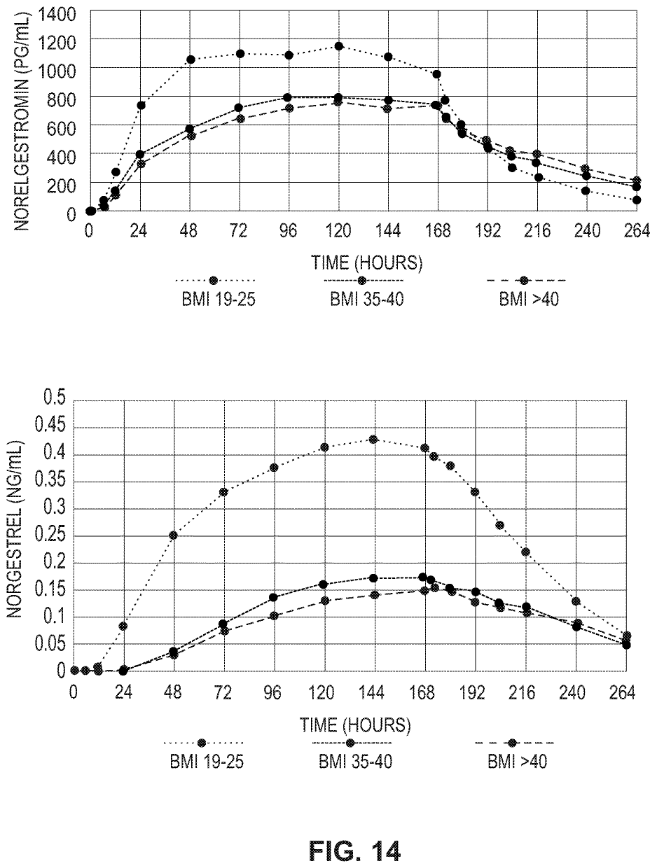

FIG. 14 illustrates a graph of the single-dose pharmacokinetics and adhesive properties of a norelgestromin transdermal system, 4.86 mg/14 cm² following a single application on each of the proposed BMI Groups, which included BMI 19-25, BMI 35-40 and BMI>40 for 7 days under normal conditions. The delivery utilizes the transdermal system having the matrix with a combination of two different PIB mixtures. In this embodiment, the patch applied having norelgestromin arrives at the highest peak at 1283 pg/mL for the BMI 19-25 group and the norgestrel at the highest peak at 0.493 ng/mL for the BMI 19-25 group. In various embodiments, the norgestrel metabolite concentration was also measured.

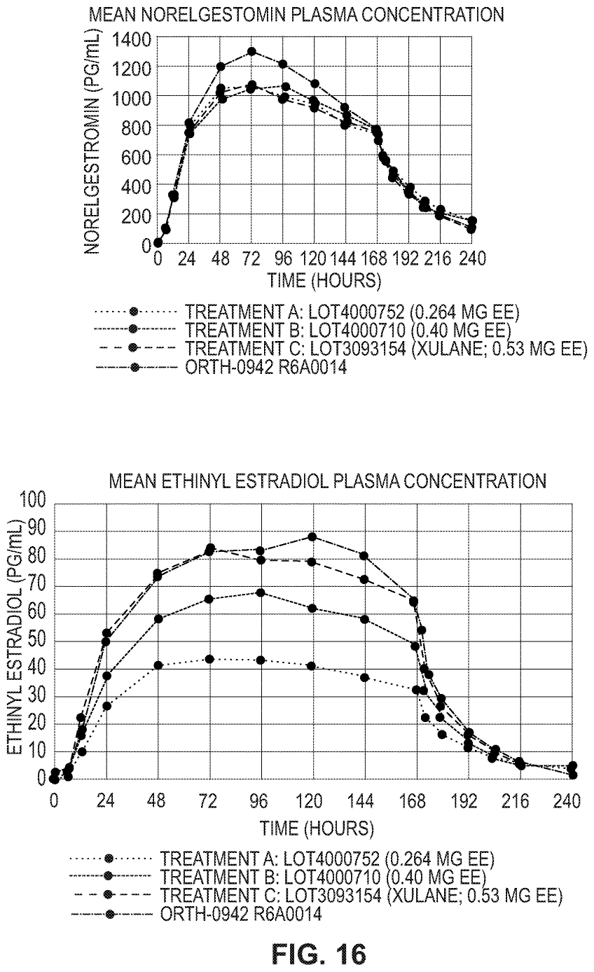

FIG. 16 illustrates a single-dose comparative bioavailability study of norelgestromin 4.86 mg/ethinyl estradiol 0.264 mg transdermal system, norelgestromin 4.86 mg/ethinyl estradiol 0.40 mg transdermal system, XULANE® transdermal system (norelgestromin 4.86 mg/ethinyl estradiol 0.53 mg) and Ortho Evra transdermal system following a 7-day wear period in healthy adult female volunteers. The testing patch with low dose of 0.264 mg ethinyl estradiol in Treatment A has achieved 100% target delivery of norelgestromin and 50% target delivery of ethinyl estradiol. These patches had single adhesives comprising two polymeric components.

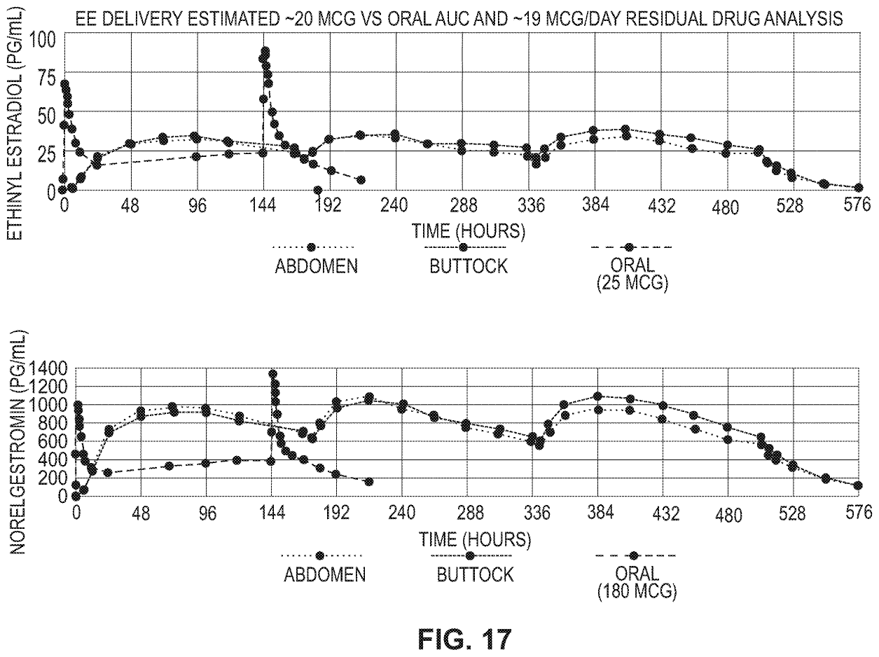

FIG. 17 illustrates a study to evaluate the multiple-dose pharmacokinetics of a commercially marketed oral product, Tri-Lo-Marzia™ Tablets versus a norelgestromin (NGMN) 4.86 mg/ethinyl estradiol (EE) 0.264 mg transdermal systems following 3 patch applications over a 21-day period at two application sites. In this embodiment, the patch applied having norelgestromin arrives at the highest peak of 1191.9 pg/mL on the buttock sites and the ethinyl estradiol at the highest peak of 43.46 pg/mL on the buttock sites. Each patch had a single adhesive comprising more than one PIB mixtures.

In many aspects, the transdermal system described in this application is applied to the skin of the patient in a regimen comprising application of one transdermal system once each week for three consecutive weeks. In other aspects, the transdermal system is applied to skin of the patient in a regimen comprising application of one transdermal system once each week for three consecutive weeks, followed by one week in which the transdermal system is not applied.

In some embodiments, the transdermal system of the current application has a favorable tolerability profile and has little or no severe local site reactions (e.g., less than 1% or 2%) from the transdermal system being worn. Severe local site reactions, include but are not limited to, blistering, crusting, or scaling of the skin at the application site; redness spreading significantly beyond the patch site (e.g., larger than your fist or more than 2 inches across), severe pain, swelling, or bruising at the site; and/or bleeding from the application area.

In some embodiments, the transdermal system of the current application has a favorable tolerability profile and has little or no severe adverse reactions (e.g., less than 1% or 2%) that extend beyond the local site reactions. Severe adverse reactions from the transdermal system can extend beyond the skin and include, but are not limited to, bleeding, blood clots (e.g., deep vein thrombosis, or pulmonary embolism), heart attack, stroke, severe stomach pain, and/or allergic reactions involving symptoms beyond the application site, like hives, swelling of the face or throat, and/or difficulty breathing.

### Methods of Making

In some embodiments, the transdermal systems of the current application can be made by preparing the adhesive and then dissolving or suspending the one or more active pharmaceutical ingredients in the adhesive to form the matrix. The matrix can then be coated on a fabric (e.g., non-woven polyester) to make a drug release layer which can be attached to the release liner, dried and then the other side of the fabric laminated to the desired backing, according to predetermined parameters, such as temperature and dwell time (line speed), which yield minimal residual solvent levels. Alternatively, the matrix can be formed and then coated on the fabric, which then can be laminated to the backing on one side and the other side of the fabric can be attached to the release liner.

In one embodiment, a method is provided of making a transdermal system comprising a backing layer, an adhesive layer comprising a therapeutic agent, and a protective release liner. In some embodiments, the therapeutic agent can be in crystalline form and/or partially dissolved, which allows it to eventually pass through the skin of a user. First, a drug reservoir adhesive layer is prepared by dissolving the therapeutic agent and other nonactive ingredients in a solvent to form a uniform solution and mixing the solution with an adhesive or adhesive solution to form a new solution or suspension and then coating a release liner or backing with the solution or suspension. The solution or suspension may also contain optional ingredients such as a penetration enhancer. The coated release liner is then dried to form a dry adhesive. The dry adhesive is then laminated to a backing film to form the three-layered film. Individual patches containing the three layers are die-cut from the laminate. Standard methods known in the art can be used to die-cut the layers from the laminate.

In another embodiment, there is a method of making a transdermal system comprising a drug reservoir layer, and a skin contact layer, each comprising a therapeutic agent and other nonactive components, a backing layer, and a protective release liner. First, a drug reservoir adhesive layer on a release liner is prepared. A drug reservoir adhesive layer is prepared by completely dissolving both the therapeutic agent and the polymeric stabilizer in a solvent to form a uniform solution and mixing the solution with an adhesive or adhesive solution to form a new solution or suspension and then coating a release liner or backing with the solution or suspension. The solution or suspension may also contain optional ingredients such as a penetration enhancer. The coated release liner is then dried to form a dry adhesive. The dry adhesive is then laminated to a backing film to form the three-layered film.

In one particular embodiment, there is a method of making a transdermal system comprising a backing layer, a release liner, and a matrix disposed between the backing layer and the release liner, the method comprising applying to the matrix an adhesive, the adhesive comprising a medium molecular weight polyisobutylene having an average molecular weight of 55,000 to 100,000, a high molecular weight polyisobutylene having an average molecular weight of 500,000 to 1,400,000 or both a medium molecular weight polyisobutylene having an average molecular weight of 55,000 to 100,000 and a high molecular weight polyisobutylene having an average molecular weight of 500,000 to 1,500,000.

In one more particular embodiment, there is a method of making a transdermal system, the method comprising mixing about 2.2 mg to about 5.3 mg of progestin per 7 to 14 cm² and applying it to a matrix, the matrix comprising an adhesive comprising a medium molecular weight polyisobutylene having an average molecular weight of 55,000 to 100,000, a high molecular weight polyisobutylene having an average molecular weight of 500,000 to 1,400,000 or both a medium molecular weight polyisobutylene having an average molecular weight of 55,000 to 100,000 and a high molecular weight polyisobutylene having an average molecular weight of 500,000 to 1,500,000, wherein the transdermal system is configured to release about 108 mcg per day to about 253 mcg per day of the progestin to a patient.

In some embodiments, the progestin comprises norelgestromin. In some embodiments, the transdermal system further comprises estrogen and the estrogen is also mixed with the matrix. In other embodiments, the transdermal system comprises the estrogen, which is ethinyl estradiol and is released from the transdermal system in an amount of about 16 mcg per day to about 23 mcg per day. In one embodiment, the ethinyl estradiol is in the transdermal system in an amount of about 0.264 mg. In some embodiments, the transdermal system is applied to a human patient.

### EXAMPLES

The following examples further illustrate the application. These examples are not intended to limit the application in any manner. Unless indicated otherwise, stated percentages are by weight.

Some embodiments of the low dose transdermal patches or films described in this application are given in Examples 1-16.

### Example 1

### Adhesion/Elegance Study

This example provides the adhesion/elegance study of different transdermal systems in patch form including a reference sample compared to a currently marketed commercial product, XULANE®. XULANE® also contains norelgestromin (4.86 mg, 2.31% ww) and ethinyl estradiol (0.53 mg, 0.25% ww). There are six patches used in the study. Unit A is the commercially marketed product, XULANE®. The components of the formulations are included in Table 1. Unit A has a 1 mil light pigmented layer PIB Y Adhesive B-Series (84% B10/16% B100). Unit B has a 1 mil darker pigmented layer PIB Y Adhesive N-Series (PIB Y: 84% B10/16% N100). Unit C has a 1.5 mil darker pigmented layer PIB Y Adhesive N-Series (PIB Y: 84% B10/16% N100). Unit D has a 2 mil darker pigmented layer PIB Y Adhesive N-Series (PIB Y: 84% B10/16% N100). Unit E has a 1 mil darker pigmented layer PIB Y Adhesive N-Series (PIB Y: 84% B10/16% N100) & 10%/PIB X Adhesive N-Series (PIB X: 55% B12/22.5% N80/22.5%). The combined polyisobutylene composition for PIB Y/PIB X @90/10 Ratio is 75.6% B10/5.5 B12/2.25% N80/16.65% N100. Unit F has a 1 mil darker pigmented layer PIB Y Adhesive N-Series (PIB Y: 84% B10/16% N100). The details of the matrix is shown in Table 1.

### Table 1-1: Adhesion/Elegance Study of Example 1

| | Formulation Name | | | | | | | | | | | |
|---|---|---|---|---|---|---|---|---|---|---|---|---|
| | Treatment A | | | | | | | | | | | |
| | (XULANE ®) | | Treatment B | | Treatment C | | Treatment D | | Treatment E | | Treatment F | |
| | Patch Size | | | | | | | | | | | |
| | 140 cm² | | 140 cm² | | 140 cm² | | 140 cm² | | 140 cm² | | 140 cm² | |
| | Matrix | | | | | | | | | | | |
| | | % | | % | | % | | % | | % | | % |
| | Mg | ww | Mg | ww | Mg | ww | Mg | ww | Mg | ww | Mg | ww |
| Dipropylene | 1.58 | 0.75 | 1.61 | 0.77 | 1.61 | 0.77 | 1.61 | 0.77 | 1.61 | 0.77 | 1.61 | 0.77 |
| Glycol LO+ | | | | | | | | | | | | |
| Crospovidone | 47.5 | 22.62 | 48.7 | 23.19 | 48.7 | 23.19 | 48.7 | 23.19 | 48.7 | 23.19 | 42 | 20 |
| Oleyl Alcohol | 7.45 | 3.55 | 7.65 | 3.64 | 7.65 | 3.64 | 7.65 | 3.64 | 7.65 | 3.64 | 7.65 | 3.64 |
| Light Mineral | 26.4 | 12.57 | 27.07 | 12.89 | 27.07 | 12.89 | 27.07 | 12.89 | 27.07 | 12.89 | 28.28 | 13.47 |
| Oil NF | | | | | | | | | | | | |
| Polyisobutylene | 121.68 | 57.95 | 125 | 59.51 | 125 | 59.51 | 125 | 59.51 | 112.4 | 53.54 | 130.5 | 62.13 |
| Adhesive (PIB | | | | | | | | | | | | |
| Y) (Second | | | | | | | | | | | | |
| Component of | | | | | | | | | | | | |
| PIB adhesive) | | | | | | | | | | | | |
| Polyisobutylene | N/A | N/A | N/A | N/A | N/A | N/A | N/A | N/A | 12.5 | 5.97 | N/A | N/A |
| (PIB X) (First | | | | | | | | | | | | |
| Component of | | | | | | | | | | | | |
| PIB adhesive) | | | | | | | | | | | | |
| Total Matrix | 150 | 150 | 150 | 150 | 150 | 150 | 150 | 150 | 150 | 150 | 150 | 150 |
| Basis Weight | g/m² | g/m² | g/m² | g/m² | g/m² | g/m² | g/m² | g/m² | g/m² | g/m² | g/m² | g/m² |

Other Components

| | | | | | | |
|---|---|---|---|---|---|---|
| Backing Film | MEDIFLEX ® | MEDIFLEX ® | MEDIFLEX ® | MEDIFLEX ® | MEDIFLEX ® | MEDIFLEX ® |
| (14 cm²) | 1501 | 1510 | 1511 | 1512 | 1510 | 1510 |
| Release Liner | Scotchpak ™ | Scotchpak ™ | Scotchpak ™ | Scotchpak ™ | Scotchpak ™ | Scotchpak ™ |
| (20.45 cm²) | 1022 | 1022 | 1022 | 1022 | 1022 | 1022 |
| Nonwoven | Reemay 2250 | Reemay 2250 | Reemay 2250 | Reemay 2250 | Reemay 2250 | Reemay 2250 |
| polyester | 0.05 OSY | 0.05 OSY | 0.05 OSY | 0.05 OSY | 0.05 OSY | 0.05 OSY |
| (14 cm²) | | | | | | |

### Table 1-2: Dry Weight Composition of Polyisobutylene Mixture

| Polyisobutylene | Polyisobutylene (PIB Y) | Polyisobutylene (PIB X) | |
|---|---|---|---|
| Homopolymer | (Second Component of | (First Component of PIB | 9:1 Ratio² |
| Component | PIB adhesive) | adhesive) | PIB Y:PIB X |
| N-100 (Avg MW = | 16% | 22.5% | 16.65% |
| 1.11M)¹ | | | |
| N-80 | N/A | 22.5% | 2.25% |
| (Avg MW = 800K)¹ | | | |
| B-12 | N/A | 55% | 5.5% |
| (Avg MW = 55K)¹ | | | |
| B-10 SFN | 84% | N/A | 75.6 |
| (Avg MW = 40K)¹ | | | |

¹Average Molecular Weight (viscosity average)
²Selected ratio of the two PIB adhesives for the investigational product

In this study, the backing film has an area of 14 cm² with release liner at 20.45 cm²; and nonwoven polyester has an area of 14 cm², which is the same size as the backing film.

The transdermal systems of the application may be fabricated using conventional procedures in the transdermal delivery system art. The procedure will generally involve formulating the matrix (i.e., mixing the adhesive, drug(s), permeation enhancer, and additives, if any), casting the matrix onto a middle layer, backing or release liner layer, removing solvent from the matrix and applying the matrix to a middle layer, backing and/or release liner layer as the case may be. As is apparent to those of ordinary skill in the art, the matrix composition has an effective amount of the drug dispersed therein, which can be incorporated into various transdermal constructions and therefore, Applicants are not limited to the embodiments exemplified below.

The method of manufacture of the low dose transdermal films of Example 1 includes: (1) dispensing and mixing; (2) first-pass coating; (3) second pass coating; (4) slitting; and (5) die cutting and packaging.

The dispensing and mixing process involves making two identical blends of the active and inactive ingredients. One blend is coated to manufacture the first pass laminate, and the second blend is coated to manufacture the second pass laminate.

The first pass laminate and the second pass laminate are coated in a two-pass configuration where the first pass is laminated to the second pass, resulting in a bilayer laminate. Optionally, a scrim, optionally made from non-woven polyester, may be used in between the first pass coating and second pass coating.

Slitting of the bilayer laminate is performed by unwinding the bilayer laminate through a set of knives and rewinding to create several narrow, slit rolls of bilayer laminate. Optionally, knife spacing is adjusted to provide the desired final dimensions for further processing. Slitting is a common process, and standard slitting conditions known to a person of skill in the art can successfully be used in the manufacture of this prototypical patch.

The die cutting operation determines the patch size and thus the dosage of the finished drug product. The parameter for the die-cutting step is patch dimension (length x width). Material thickness and physical properties of the backing are known to affect the performance of the kiss-cut die during packaging.

The backing used for the patches of Example 1 was pre-printed. Thus, the printing unit operation was not necessary for the production of Example 1. However, the use of such an operation is well known in the art.

### Example 2

This example provides the adhesion results from applying the matrix shown in the Example 1. In the study, there are 36 female subjects with 12 patients per Fitzpatrick Skin Type 1&2, Type 3&4 and Type 5&6. Type 1: N=1, Type 2: N=11, Type 3: N=5, Type 4: N=7, Type 5: N=12, Type 6: N=0 (N=36 patients) All 6 patches are worn simultaneously for 7 days (168 hours). The application sites for each were randomized among left and right abdomen; left and right buttock; and left and right upper back. The adhesion is evaluated every 24 hours. The visual evaluation of the percent unit, which adheres to application site is evaluated from 100% to 0%, which is known as adhesion rate/fall off rate. The percent score is then converted to FDA adhesion scale (0, 1, 2, 3, 4). Adhesion performance of the transdermal patches was evaluated using a frequency analysis of patch adhesion scores for each treatment arm (Unit A to Unit F). Assessments were conducted for each day of the application period of a single patch, from Day 1 through Day 7. In addition, subject-wise mean adhesion scores and maximum adhesion scores were calculated over the entire one-week application period for each patch. For each treatment arm and each evaluation time point (day or week, as applicable), the proportion of subjects achieving greater than 90% patch adherence (corresponding to an adhesion score of 0) was determined. Further analyses included the proportion of subjects exhibiting a clinically meaningful degree of patch detachment, defined as detachment of at least 25% of the patch surface area (estimated adhesion score ≥2), as well as the total number of patches that became completely detached (adhesion score of 4).

The comparison of all units compared to XULANE® shows a ranking of 1. Unit E, 2. Unit D, 3. Unit C, 4. Unit B, 5. Unit F. The Unit E, D and C had better cumulative adhesion scores compared to Unit A, as shown in Table 2-1 and 2-2 below.

### Table 2-1

| | | Standard | | | | |
|---|---|---|---|---|---|---|
| Parameter | Estimate | Error | t Value | Pr > \|t\| | 90% Confidence Limits | |
| B vs A | 0.03968254 | 0.12449439 | 0.32 | 0.7503 | −0.16621454 | 0.24557962 |
| C vs A | −0.05158730 | 0.12449439 | −0.41 | 0.6791 | −0.25748438 | 0.15430978 |
| D vs A | −0.08730159 | 0.12449439 | −0.70 | 0.4841 | −0.29319867 | 0.11859550 |
| E vs A | −0.16666667 | 0.12449439 | −1.34 | 0.1824 | −0.37256375 | 0.03923042 |
| F vs A | 0.14285714 | 0.12449439 | 1.15 | 0.2528 | −0.06303994 | 0.34875423 |

For assessment of adhesion, the observed percent (%) adhered of each patch was recorded. Percent adhered scores were then converted to the FDA (0-4) Scale for statistical analysis. The FDA's October 2018 Draft Adhesion Guidance non-inferiority criteria were used. The null and alternative hypotheses were: H0: μ1-μ2≥0.15 and H1: μ1-μ2<0.15, where μ1 is the mean adhesion score for the test product and μ2 is the mean adhesion score for the reference product. The null hypothesis H0 was rejected when the upper limit of the 90% confidence interval (that is the one-sided 95% upper confidence bound) for the quantity μ1-μ2 was <0.1500. In Table 2-1, Estimate is μ1-μ2 representing the least-squares mean of the adhesion score for a Unit subtracts the least-squares mean of the adhesion score for a reference Unit. For example, for B vs A, arithmetic mean (% cv) of adhesion scores observed in healthy adult female subjects following a single application of each transdermal system worn for 7 days for Unit B has a cumulative mean of 0.254 and Unit A has a cumulative mean of 0.2143. The difference is 0.0397 and 95% confidence interval is 0.2456.

### Table 2-2

| | score1 |
|---|---|
| treat | LSMEAN |
| A | 0.21428571 |
| B | 0.25396825 |
| C | 0.16269841 |
| D | 0.12698413 |
| E | 0.04761905 |
| F | 0.35714286 |

Table 2-2 shows cumulative mean of the least-square mean calculated over 7 days for each treatment unit. A least-squares mean (often called a LS mean, and also known as a model-adjusted mean) is a statistical estimate of a group's mean that has been adjusted for other variables included in a statistical model. Therefore, Unit E has an adjusted mean closest to an adhesion score of 0, which means that Unit E exhibits improved adhesion, for example in sauna and exercise conditions, and achieves greater than 90% patch adherence.

### Example 3

This example provides various tables showing adhesion rate/fall off rate of different matrices used in the transdermal system shown in the Example 1, Unit A to Unit F applying to various parts of the subject's body.

Table 3-1 shows the overall score for all units with combined left and right sides, as illustrated in FIG. 1.

Overall

| | Unit A | Unit B | Unit C | Unit D | Unit E | Unit F |
|---|---|---|---|---|---|---|
| Time | XULANE | 4001398 | 4001399 | 4001400 | 4001416 | 4001439 |
| 0 | 100 | 100 | 100 | 100 | 100 | 100 |
| 24 | 100 | 99 | 100 | 100 | 100 | 100 |
| 48 | 99 | 98 | 98 | 100 | 99 | 100 |
| 72 | 96 | 97 | 98 | 96 | 99 | 99 |
| 96 | 92 | 94 | 97 | 96 | 98 | 93 |
| 120 | 91 | 93 | 96 | 94 | 99 | 83 |
| 144 | 89 | 88 | 93 | 94 | 99 | 82 |
| 168 | 89 | 88 | 88 | 94 | 93 | 77 |

After six days Unit E is consistent and maintains 99% of the adhesion rate. After seven days of application, Unit D shows 94% and Unit E drops to 93% of the adhesion rate.

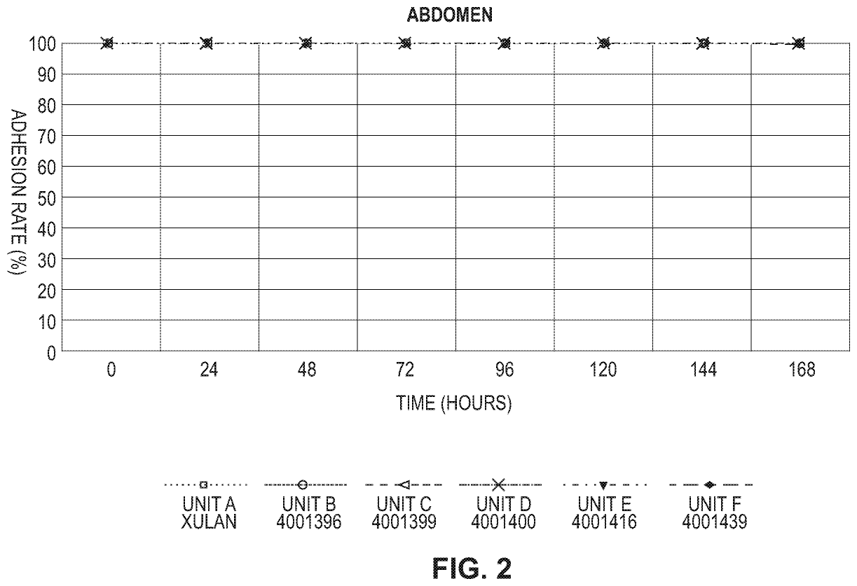

Table 3-2 illustrates the adhesives on the abdomen with combined left and right sides, as illustrated in FIG. 2.

Abdomen

| | Unit A | Unit B | Unit C | Unit D | Unit E | Unit F |
|---|---|---|---|---|---|---|
| Time | XULANE | 4001398 | 4001399 | 4001400 | 4001416 | 4001439 |
| 0 | 100 | 100 | 100 | 100 | 100 | 100 |
| 24 | 100 | 100 | 100 | 100 | 100 | 100 |
| 48 | 100 | 100 | 100 | 100 | 100 | 100 |
| 72 | 100 | 100 | 100 | 100 | 100 | 100 |
| 96 | 100 | 100 | 100 | 100 | 100 | 100 |
| 120 | 100 | 100 | 100 | 100 | 100 | 100 |
| 144 | 100 | 100 | 100 | 100 | 100 | 100 |
| 168 | 100 | 100 | 100 | 100 | 100 | 100 |

There are no falloffs observed from the abdomen.

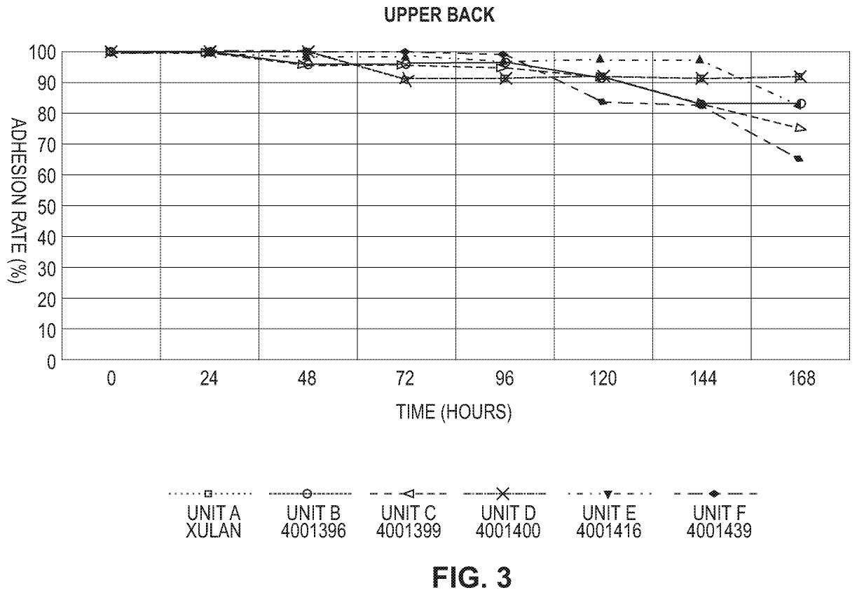

Table 3-3 illustrates the adhesives on the upper back with combined left and right sides, as illustrated in FIG. 3. Unit E has the most consistent wear rate over the 7 days compared to the other transdermal systems.

Upper Back

| | Unit A | Unit B | Unit C | Unit D | Unit E | Unit F |
|---|---|---|---|---|---|---|
| Time | XULANE | 4001398 | 4001399 | 4001400 | 4001416 | 4001439 |
| 0 | 100 | 100 | 100 | 100 | 100 | 100 |
| 24 | 100 | 100 | 100 | 100 | 100 | 100 |
| 48 | 100 | 96 | 96 | 100 | 99 | 100 |
| 72 | 91 | 96 | 96 | 91 | 99 | 100 |
| 96 | 91 | 96 | 95 | 91 | 97 | 99 |
| 120 | 91 | 92 | 92 | 92 | 98 | 83 |
| 144 | 92 | 83 | 83 | 91 | 98 | 82 |
| 168 | 92 | 83 | 75 | 92 | 83 | 65 |

The falloffs on the upper back shows Unit A=1, Unit B=2, Unit C=2, Unit D=1, Unit E=0, and Unit F=4.

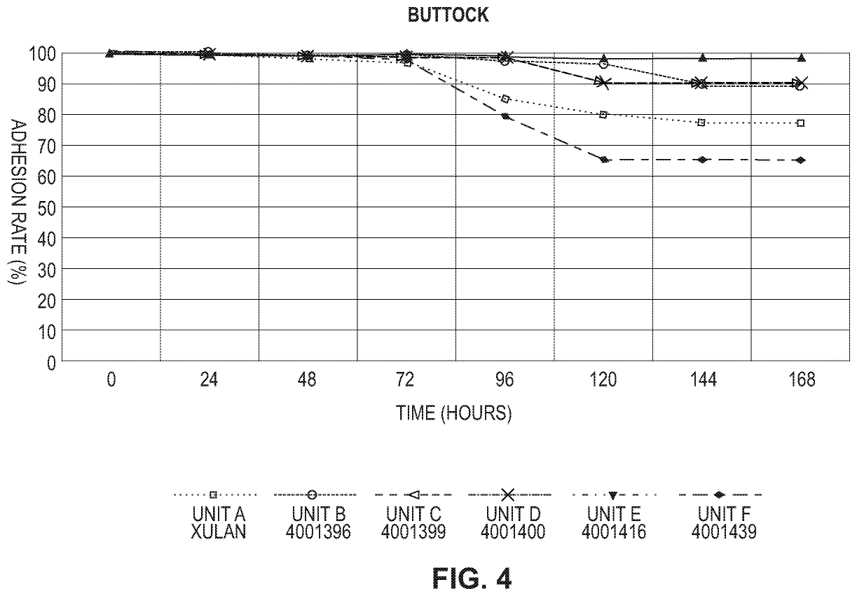

Table 3-4 presents the adhesives on the buttock with combined left and right sides, as illustrated in FIG. 4. Unit E has the most consistent wear rate over the 7 days compared to the other transdermal systems as indicated by the higher linear line.

Buttock

| | Unit A | Unit B | Unit C | Unit D | Unit E | Unit F |
|---|---|---|---|---|---|---|
| Time | XULANE | 4001398 | 4001399 | 4001400 | 4001416 | 4001439 |
| 0 | 100 | 100 | 100 | 100 | 100 | 100 |
| 24 | 99 | 98 | 100 | 100 | 99 | 100 |
| 48 | 98 | 98 | 99 | 99 | 99 | 99 |
| 72 | 97 | 95 | 99 | 98 | 99 | 97 |
| 96 | 85 | 87 | 97 | 98 | 99 | 79 |
| 120 | 80 | 88 | 96 | 90 | 98 | 65 |
| 144 | 77 | 80 | 98 | 90 | 98 | 65 |
| 168 | 77 | 80 | 89 | 90 | 98 | 65 |

The falloffs on upper back shows Unit A=2, Unit B=2, Unit C=2, Unit D=1, Unit E=2, and Unit F=4.

Table 3-4 illustrates the dermal response or clinical irritation results from all wear sites combined left and right sides. The acute irritation (30 minutes post removal) was similar and mild across all treatments.

| All Patches: | Dermal Response | | Dermal Response + Other Effects | |
|---|---|---|---|---|
| Treatment | Mean | S.D. | Mean | S.D. |
| A = XULANE ® | 0.694 | 0.822 | 0.694 | 0.822 |
| B = 4001398 | 0.611 | 0.838 | 0.611 | 0.838 |
| C = 4001399 | 0.583 | 0.806 | 0.583 | 0.806 |
| D = 4001400 | 0.500 | 0.775 | 0.500 | 0.775 |
| E = 4001416 | 0.694 | 0.951 | 0.694 | 0.951 |
| F = 4001439 | 0.694 | 0.822 | 0.722 | 0.882 |

Tables 3-5 illustrates the dermal response or clinical irritation results from all wear sites combined left and right sides excluding the fall offs.

| Excluding | | | | |
|---|---|---|---|---|
| Fall Offs: | Dermal Response | | Dermal Response + Other Effects | |
| Treatment | Mean | S.D. | Mean | S.D. |
| A = XULANE ® | 0.727 | 0.839 | 0.727 | 0.839 |
| B = 4001398 | 0.656 | 0.865 | 0.656 | 0.865 |
| C = 4001399 | 0.656 | 0.827 | 0.656 | 0.827 |
| D = 4001400 | 0.529 | 0.788 | 0.529 | 0.788 |
| E = 4001416 | 0.706 | 0.970 | 0.706 | 0.970 |
| F = 4001439 | 0.679 | 0.863 | 0.714 | 0.937 |

The study shows hat Units C and D appear to mask the wear discoloration under the backing film better than units A, B, E, and F. The buttock wear site appears to increase the chances of unit buckling, which would lead to a decrease in unit wear area, and possible unit failure.

Overall, all formulations of the XULANE® Placebo Transdermal Systems and XULANE® Transdermal System were well tolerated as a single, transdermal patch application worn for 7 days. The rank order of the cumulative mean adhesion scores from highest to lowest were: Treatment F>Treatment B>Treatment A>Treatment C>Treatment D>Treatment E. The results of the statistical analysis indicate that the null hypothesis (H0: μ1-μ2≥0.15) is rejected for Treatment D and Treatment E when compared to Treatment A. Treatment C was just slightly above the non-inferiority limit with an upper bound of the 95% confidence interval of 0.1543. The upper bound of the 95% confidence interval for Treatment B and Treatment F relative to Treatment A was >0.15. Thus, adhesion for Treatment D and Treatment E is no worse than (i.e., non-inferior to) XULANE® adhesion over the intended 7-day application period.

### Example 4

This example provides various tables comparing the contraceptive agent norelgestromin and ethinyl estradiol using Unit E at various wear sites, as illustrated in FIG. 5.

Table 4-1 illustrates the amount of norelgestromin at 4 different application sites.

Arithmetic Mean (% CV) Norelgestromin Pharmacokinetic Parameters

| | Abdomen | | Upper Back | | Upper Outer Arm | | Buttock | |
|---|---|---|---|---|---|---|---|---|
| Parameter | (n = 29) | | (n = 29) | | (n = 27) | | (n = 29) | |
| AUCINF (ng*hr/mL) | 207.97 | (35.0%) | 216.10 | (34.7%) | 217.04 | (31.1%) | 186.06 | (32.0%) |
| AUCL (ng*hr/mL) | 201.04 | (33.5%) | 209.34 | (33.0%) | 210.47 | (30.7%) | 180.09 | (30.8%) |
| CPEAK (ng/mL) | 1.276 | (32.5%) | 1.367 | (30.7%) | 1.369 | (31.9%) | 1.197 | (33.5%) |
| KEL (1/hr) | 0.0226 | (31.2%) | 0.0229 | (32.2%) | 0.0233 | (35.1%) | 0.0231 | (34.9%) |
| HALFLIFE (hours) | 33.32 | (29.0%) | 33.49 | (33.1%) | 33.25 | (34.0%) | 33.57 | (35.4%) |
| TPEAK (hours) | 89.53 | (30.4%) | 82.76 | (36.0%) | 81.85 | (36.6%) | 99.38 | (32.1%) |
| Daily Dose (mcg/day) | 220 | (18.3%) | 239 | (23.1%) | 214 | (23.7%) | 226 | (31.4%) |

Table 4-2 illustrates the amount of ethinyl estradiol at 4 different application sites.

Arithmetic Mean (% CV) Ethinyl Estradiol Pharmacokinetic Parameters

| | Abdomen | | Upper Back | | Upper Outer Arm | | Buttock | |
|---|---|---|---|---|---|---|---|---|
| Parameter | (n = 28) | | (n = 28) | | (n = 26) | | (n = 28) | |
| AUCINF (pg*hr/mL) | 5205.7 | (29.5%)¹ | 6192.8 | (29.7%)¹ | 5240.3 | (28.7%) | 4992.2 | (35.6%) |
| AUCL (pg*hr/mL) | 5194.5 | (29.2%) | 6097.8 | (29.8%) | 5193.2 | (29.0%) | 4944.1 | (35.9%) |
| CPEAK (pg/mL) | 38.00 | (30.7%) | 45.58 | (35.3%) | 38.25 | (31.6%) | 37.51 | (40.2%) |
| KEL (1/hr) | 0.0373 | (32.6%)¹ | 0.0353 | (27.8%)¹ | 0.0373 | (37.1%) | 0.0372 | (27.4%) |
| HALFLIFE (hours) | 20.27 | (27.9%)¹ | 21.09 | (27.6%)¹ | 20.36 | (27.6%) | 19.92 | (25.4%) |
| TPEAK (hours) | 93.43 | (39.8%) | 81.55 | (35.4%) | 83.08 | (48.5%) | 96.86 | (37.8%) |
| Daily Dose (mcg/day) | 21 | (21.8%) | 23 | (19.1%) | 19 | (22.4%) | 21 | (29.8%) |

¹n = 27

Table 4-3 illustrates the summary of patch adhesion.

Summary of Patch Adhesion

| | Abdomen | Upper Back | Upper Outer | Buttock |
|---|---|---|---|---|
| Application Site | (n = 31) | (n = 31) | Arm (n = 30) | (n = 31) |
| Cumulative Mean Score (% CV) | 97.49 (7.65%) | 95.69 (16.55%) | 92.14 (23.68%) | 98.75 (2.27%) |
| Number of Patches >75% Adhered | 29 | 30 | 27 | 31 |
| Detachments Reported | 2 | 1 | 3 | 0 |

This study demonstrates the rate and extent of norelgestromin absorption across the 4 application sites that were investigated. It will be understood that any of these sites can be used to effectively deliver one or more active ingredients.

### Example 5

This example provides various tables comparing the contraceptive agents norelgestromin and ethinyl estradiol using Unit E under different wear conditions, as illustrated in FIG. 6.

For normal conditions, subjects performed routine daily activities and avoided bathing, soaking in water, or inducing excessive sweating. For cool water soaking conditions, subjects soaked once daily in a tub of cool water maintained at a temperature between 20 °C and 22 °C (68.0 °F to 71.6 °F) for a minimum of 5 minutes and no more than 15 minutes during the 7-day patch wear period under supervision of clinical staff. For whirlpool conditions, subjects soaked once daily in a whirlpool with a water temperature between 39 °C and 41 °C (102 °F to 106 °F) for 10 minutes during the 7-day patch wear period under clinical staff supervision. A 28-day washout period separated patch applications in each study period. Adhesion and patch-print legibility were evaluated once every 24 hours throughout each 7-day wear period.

Table 5-1 illustrates amount of norelgestromin at 3 different wear conditions.

Arithmetic Mean (% CV) Norelgestromin
Pharmacokinetic Parameters

| | Cool Water | | Whirlpool | | Normal | |
|---|---|---|---|---|---|---|
| Parameter | (n = 24) | | (n = 25) | | (n = 24) | |
| AUCINF | 211.991 | (38.2%) | 212.266 | (28.6%) | 218.044 | (33.5%) |
| (ng*hr/mL) | | | | | | |
| AUCL | 205.618 | (38.2%) | 205.687 | (28.6%) | 211.574 | (33.4%) |
| (ng*hr/mL) | | | | | | |
| CPEAK | 1.395 | (33.8%) | 1.388 | (29.4%) | 1.439 | (29.5%) |
| (ng/mL) | | | | | | |
| KEL (1/hr) | 0.0216 | (21.2%) | 0.0213 | (21.3%) | 0.0216 | (19.8%) |
| HALFLIFE | 33.553 | (22.3%) | 34.004 | (21.4%) | 33.241 | (18.3%) |
| (hours) | | | | | | |
| TPEAK | 78.875 | (41.2%) | 78.773 | (33.8%) | 84.403 | (30.7%) |
| (hours) | | | | | | |
| Daily Dose | 187 | (26.4%) | 187 | (19.9%) | 197 | (27.6%) |
| (mcg/day) | | | | | | |

Table 5-2 illustrates amount of ethinyl estradiol at 3 different wear conditions.

Arithmetic Mean (% CV) Ethinyl Estradiol Pharmacokinetic Parameters

| | Cool Water | | Whirlpool | | Normal | |
|---|---|---|---|---|---|---|
| Parameter | (n = 24) | | (n = 24) | | (n = 23) | |
| AUCINF (pg*hr/mL) | 4004.002 | (30.4%)¹ | 3629.087 | (35.7%) | 3734.903 | (36.0%)¹ |
| AUCL (pg*hr/mL) | 3672.170 | (41.3%) | 3585.393 | (35.9%) | 3645.325 | (36.6%) |
| CPEAK (pg/mL) | 30.035 | (47.0%) | 27.002 | (37.5%) | 27.603 | (39.8%) |
| KEL (1/hr) | 0.0363 | (19.8%)¹ | 0.0367 | (26.9%) | 0.0366 | (24.8%)¹ |
| HALFLIFE (hours) | 19.828 | (20.2%)¹ | 19.786 | (19.2%) | 19.939 | (23.0%)¹ |
| TPEAK (hours) | 99.611 | (45.7%) | 81.639 | (41.8%) | 95.391 | (39.9%) |
| Daily Dose (mcg/day) | 17 | (34.7%) | 17 | (24.8%) | 17 | (34.5%) |

¹n = 22

Table 5-3 illustrates the summary of patch adhesion.

Summary of Patch Adhesion

| | Cool Water | Whirlpool | Normal |
|---|---|---|---|
| Application Site | (n = 31) | (n = 28) | (n = 28) |
| Cumulative Mean Score (% CV) | 96.44 (8.90%) | 97.15% (11.56%) | 98.32% (6.04%) |
| Number of Patches >75% Adhered | 28 | 27 | 27 |
| Detachments Reported | 3 | 1 | 1 |

This study demonstrates the rate and extent of norelgestromin absorption was similar across the three treatment conditions that were investigated.

### Example 6

This example provides various tables comparing the contraceptive agents norelgestromin and ethinyl estradiol using Unit E under different wear conditions, as illustrated in FIG. 7.

This was a single-dose, randomized, three-period, three-treatment, crossover study. Subjects were housed from the evening prior to dosing and until at least 24 hours after dosing, and again from approximately 166 to 180 hours after dosing in each period. Subjects received a standard low-fat meal on the evening prior to dosing. Under sauna conditions, subjects were exposed once daily to a dry sauna environment with temperatures ranging from 76 °C to 82 °C (168.8 °F to 179.6 °F) for 10 minutes during the 7-day patch wear period under clinical staff supervision. Under exercise conditions, subjects walked on a treadmill once daily for 20 to 30 minutes at 60-80% of maximum heart rate, calculated as 220 minus the subject's age, during the 7-day patch wear period under clinical staff supervision. A 28-day washout period separated patch applications in each study period. Patch adhesion and patch-print legibility were evaluated once every 24 hours throughout each 7-day wear period.

Table 6-1 illustrates amount of norelgestromin at 3 different wear conditions.

Arithmetic Mean (% CV) Norelgestromin
Pharmacokinetic Parameters

| | Sauna | | Exercise | | Normal | |
|---|---|---|---|---|---|---|
| Parameter | (n = 30) | | (n = 29) | | (n = 29) | |
| AUCINF | 200.551 | (40.6%) | 186.484 | (37.1%) | 197.212 | (39.1%) |
| (ng*hr/mL) | | | | | | |
| AUCL | 194.178 | (40.0%) | 180.428 | (35.9%) | 190.586 | (38.3%) |
| (ng*hr/mL) | | | | | | |
| CPEAK | 1.303 | (32.4%) | 1.177 | (32.4%) | 1.263 | (33.1%) |
| (ng/mL) | | | | | | |
| KEL | 0.0225 | (27.9%) | 0.0220 | (21.8%) | 0.0222 | (23.5%) |
| (1/hr) | | | | | | |
| HALFLIFE | 33.087 | (28.3%) | 33.108 | (25.0%) | 33.241 | (27.8%) |
| (hours) | | | | | | |
| TPEAK | 89.196 | (42.7%) | 90.702 | (32.4%) | 93.299 | (39.3%) |
| (hours) | | | | | | |
| Daily Dose | 196 | (25.1%) | 196 | (28.6%) | 193 | (28.9%) |
| (mcg/day) | | | | | | |

Table 6-2 illustrates amount of ethinyl estradiol at 3 different wear conditions.

Arithmetic Mean (% CV) Ethinyl Estradiol Pharmacokinetic Parameters

| | Sauna | | Exercise | | Normal | |
|---|---|---|---|---|---|---|
| Parameter | (n = 29) | | (n = 29) | | (n = 29) | |
| AUCINF (pg*hr/mL) | 5089.770 | (37.6%) | 5098.351 | (43.0%) | 5299.994 | (44.6%) |
| AUCL (pg*hr/mL) | 5030.384 | (37.8%) | 5052.722 | (43.3%) | 5237.427 | (44.9%) |
| CPEAK (pg/mL) | 40.170 | (33.8%) | 39.161 | (41.7%) | 42.377 | (45.7%) |
| KEL (1/hr) | 0.0329 | (27.9%) | 0.0381 | (30.3%) | 0.0345 | (30.7%) |
| HALFLIFE (hours) | 22.421 | (23.3%) | 19.594 | (25.6%) | 21.824 | (28.4%) |
| TPEAK (hours) | 78.325 | (55.9%) | 92.468 | (40.3%) | 87.920 | (47.1%) |
| Daily Dose (mcg/day) | 18 | (28.1%) | 18 | (37.0%) | 17 | (34.5%) |

Table 6-3 illustrates the summary of patch adhesion.

Summary of Patch Adhesion

| | Sauna | Exercise | Normal |
|---|---|---|---|
| Application Site | (n = 32) | (n = 32) | (n = 33) |
| Cumulative Mean | 98.32 (2.43%) | 95.45 (16.75%) | 94.55 (17.99%) |
| Score (% CV) | | | |
| Number of | 32 | 30 | 30 |
| Patches >75% | | | |
| Adhered | | | |
| Detachments | 0 | 0 | 3 |
| Reported | | | |

This study demonstrates the rate and extent of norelgestromin and ethinyl estradiol absorption across the three treatment conditions that were investigated. Specifically, FIG. 7 illustrates a graph of norelgestromin in an amount of 4.86 mg and ethinyl estradiol in an amount of 0.264 mg in a transdermal system following a 7-day wear period on the abdomen in healthy adult female volunteers under normal, sauna and exercise conditions. Treatment E, under sauna conditions is able to deliver 1.303 ng/mL to the test subjects.

### Example 7

This example provides various tables comparing the delivery of the contraceptive agents norelgestromin and ethinyl estradiol using Unit E and the reference Unit A, the commercially marketed product, XULANE®.

Table 7-1 illustrates the delivery of norelgestromin in both Unit E and Unit A, as illustrated in FIG. 8.

Arithmetic Mean (% CV) Norelgestromin
Pharmacokinetic Parameters

| | Unit E | | Unit A | |
|---|---|---|---|---|
| Parameter | (n = 28) | | (n = 28) | |
| AUCINF (ng*hr/mL) | 166.331 | (27.8%) | 150.778 | (29.6%) |
| AUCL (ng*hr/mL) | 162.316 | (27.6%) | 147.111 | (29.1%) |
| CPEAK (ng/mL) | 1.0294 | (27.5%) | 0.9451 | (22.2%) |
| KEL (1/hr) | 0.0239 | (21.8%) | 0.0240 | (23.3%) |
| HALFLIFE (hours) | 30.356 | (23.0%) | 30.286 | (21.9%) |
| TPEAK (hours) | 87.429 | (33.6%) | 90.886 | (34.8%) |
| Daily Dose (mcg/day) | 187 | (22.5%) | 184 | (23.9%) |

As shown in Table 7-1, Unit E delivered a higher concentration of norelgestromin over time at about 166 ng*hr/mL, which is 10% higher than Unit A. Unit E also delivers a higher peak concentration of 1.03 ng/mL, which is about 9% higher than Unit A. This higher delivery allows Unit E to achieve a daily dosage of 187 mcg/day.

Table 7-2 illustrates the delivery of ethinyl estradiol in both Unit E and Unit A.

Arithmetic Mean (% CV) Ethinyl Estradiol
Pharmacokinetic Parameters

| | Unit E | | Unit A | |
|---|---|---|---|---|
| Parameter | (n = 27) | | (n = 27) | |
| AUCINF (pg*hr/mL) | 4203.195 | (60.7%) | 7770.913 | (42.8%) |
| AUCL (pg*hr/mL) | 3806.818 | (33.7%) | 7451.556 | (36.6%) |
| CPEAK (pg/mL) | 28.393 | (33.7%) | 54.690 | (26.2%) |
| KEL (1/hr) | 0.0438 | (31.1%) | 0.0429 | (26.8%) |
| HALFLIFE (hours) | 24.249 | (174%) | 20.673 | (113%) |
| TPEAK (hours) | 90.667 | (48.3%) | 93.333 | (43.6%) |
| Daily Dose (mcg/day) | 15 | (23.9%) | 29 | (31.0%) |

Table 7-3 illustrates the summary of patch adhesion.

Summary of Patch Adhesion

| | MR-100A-01 | XULANE ® |
|---|---|---|
| Application Site | (n = 30) | (n = 30) |
| Cumulative Mean Score (% CV) | 97.89 (2.59%) | 98.27 (1.21%) |
| Number of Patches > 75% Adhered | 29 | 30 |
| Detachments Reported | 0 | 0 |

This study demonstrates that the rate and extent of norelgestromin absorption was similar for Unit E norelgestromin 4.86 mg/ethinyl estradiol 0.264 mg Transdermal System (14 cm²) compared to XULANE® Transdermal System (norelgestromin, 4.86 mg and ethinyl estradiol, 0.53 mg/14 cm²) following a single, transdermal patch application on the lower abdomen for 7-days worn under normal conditions. The bioavailability of ethinyl estradiol was approximately 50% lower following application of Unit E norelgestromin 4.86 mg/ethinyl estradiol 0.264 mg Transdermal System compared to XULANE® Transdermal System as expected since the test product contains half of the drug load of ethinyl estradiol compared to XULANE®. In addition, the adhesion data revealed well-adhering products with cumulative mean adhesion scores for both treatments being greater than 97% adhered and all but one of the patches worn in the study had greater than a 75% adhesion score across all evaluations over the 7-day wear period.

### Example 8

This example provides various tables comparing the delivery of the contraceptive agent norelgestromin using Unit E and the commercially marketed product, EVRA® Canada.

Table 8-1 illustrates the delivery of norelgestromin in both Unit E and EVRA® Canada, as illustrated in FIG. 9.

Arithmetic Mean (% CV) Norelgestromin
Pharmacokinetic Parameters

| | | |
|---|---|---|
| Param- | Unit E | EVRA ® Canada |
| eter | (n = 32) | (n = 32) |

| | | | | |
|---|---|---|---|---|
| AUCINF (ng*hr/mL) | 147.070 | (30.4%)^{a} | 143.350 | (29.9%) |
| AUCL (ng*hr/mL) | 144.674 | (29.9%) | 140.419 | (30.0%) |
| CPEAK (ng/mL) | 0.927 | (30.8%) | 0.903 | (31.1%) |
| KEL (1/hr) | 0.0243 | (20.2%)^{a} | 0.0243 | (20.4%) |
| HALFLIFE (hours) | 29.672 | (20.1%)^{a} | 29.534 | (18.8%) |
| TPEAK (hours) | 94.500 | (41.3%) | 90.125 | (32.1%) |
| Daily Dose (mcg/day) | 184 | (26.0%) | 200 | (28.3%) |

^{a}n = 31

Table 8-2 illustrates the delivery of ethinyl estradiol in both Unit E and EVRA® Canada.

Arithmetic Mean (% CV) Ethinyl Estradiol
Pharmacokinetic Parameters

| | Unit E | | EVRA ® Canada | |
|---|---|---|---|---|
| Parameter | (n = 31) | | (n = 31) | |
| AUCINF (pg*hr/mL) | 3815.167 | (35.1%) ^{a} | 5977.298 | (40.5%) |
| AUCL (pg*hr/mL) | 3797.886 | (36.2%) | 5876.093 | (41.0%) |
| CPEAK (pg/mL) | 27.890 | (34.2%) | 41.177 | (39.2%) |
| KEL (1/hr) | 0.0468 | (29.8%) ^{a} | 0.0431 | (22.6%) |
| HALFLIFE (hours) | 15.814 | (23.9%) ^{a} | 16.794 | (19.9%) |
| TPEAK (hours) | 103.742 | (45.3%) | 94.452 | (38.2%) |
| Daily Dose (mcg/day) | 16 | (30.8%%) | 28 | (39.2%) |

^{a} n = 30

Table 8-3 illustrates the summary of patch adhesion.

Summary of Patch Adhesion

| | | EVRA ® |
|---|---|---|
| | Unit E | Canada |
| Application Site | (n = 35) | (n = 35) |
| Cumulative Mean Score (% CV) | 89.00 (23.7%) | 93.62 (4.63%) |
| Number of Patches > 75% Adhered | 33 | 33 |
| Detachments Reported | 2 | 0 |

### Example 9

This example provides various tables comparing the delivery of the contraceptive agent norelgestromin using Unit E in various sizes as illustrated in FIG. 10.

Table 9-1 illustrates the delivery of norelgestromin at 3 different sizes.

Arithmetic Mean (% CV) Norelgestromin Pharmacokinetic Parameters

| | 2.43 mg/7 cm² | | 3.64 mg/10.5 cm² | | 4.86 mg/14 cm² | |
|---|---|---|---|---|---|---|
| Parameter | (n = 29) | | (n = 30) | | (n = 32) | |
| AUCINF (pg*hr/mL) | 104185.5 | (33.3%) | 146906.8 | (28.9%) | 199446.4 | (30.5%) |
| AUCL (pg*hr/mL) | 99051.9 | (32.2%) | 140275.1 | (29.1%) | 190643.1 | (29.2%) |
| CPEAK (pg/mL) | 604.255 | (27.1%) | 868.877 | (29.7%) | 1183.078 | (27.7%) |
| KEL (1/hr) | 0.0185 | (22.6%) | 0.0187 | (25.4%) | 0.0196 | (25.9%) |
| HALFLIFE (hours) | 39.605 | (26.2%) | 39.335 | (25.0%) | 37.833 | (26.8%) |
| TPEAK (hours) | 94.345 | (35.3%) | 96.106 | (29.2%) | 92.311 | (31.7%) |
| Daily Dose (mcg/day) | 120 | (20.4%) | 170 | (23.1%) | 230 | (21.1%) |

Table 9-2 illustrates the PK parameters of norgestrel compared Unite E of various patch areas.

Arithmetic Mean (% CV) Norgestrel Pharmacokinetic Parameters

| | 2.43 mg/7 cm² | | 3.64 mg/10.5 cm² | | 4.86 mg/14 cm² | |
|---|---|---|---|---|---|---|
| Parameter | (n = 29) | | (n = 29) | | (n = 31) | |
| AUCINF (ng*hr/mL) | 38.328 | (35.0%)¹ | 41.160 | (37.6%)² | 55.604 | (39.0%)³ |
| AUCL (ng*hr/mL) | 19.065 | (76.0%) | 30.994 | (51.0%) | 44.286 | (45.1%) |
| CPEAK (ng/mL) | 0.142 | (59.5%) | 0.213 | (37.2%) | 0.276 | (40.5%) |
| KEL (1/hr) | 0.0109 | (36.9%)¹ | 0.0115 | (28.9%)² | 0.0121 | (29.9%)³ |
| HALFLIFE (hours) | 81.899 | (80.6%)¹ | 66.905 | (37.2%)² | 63.018 | (33.5%)³ |
| TPEAK (hours) | 136.661 | (17.5%) | 133.494 | (16.4%) | 143.131 | (16.1%) |

¹n = 18,
²n = 25,
³n = 29

Table 9-3 illustrates the summary of patch adhesion.

Summary of Patch Adhesion

| | 2.43 | 3.64 | 4.86 |
|---|---|---|---|
| | mg/7 cm² | mg/10.5 cm² | mg/14 cm² |
| Application Site | (n = 32) | (n = 31) | (n = 33) |
| Cumulative Mean | 96.01 (15.6%) | 98.90 (1.14%) | 98.30 (2.10%) |
| Score (% CV) | | | |
| Number of Patches > | 31 | 31 | 32 |
| 75% Adhered | | | |
| Detachments Reported | 1 | 0 | 0 |

This study demonstrates that the bioavailability of norelgestromin following a single, transdermal patch worn for 7 days was dose proportional for CPEAK, AUCL, and AUCINF across the 2.43 mg/7 cm², 3.64/10.5 cm², and 4.86 mg/14 cm² strengths of Unit E's norelgestromin transdermal system. The 90% confidence intervals of the proportionality constant B1, for LNCPEAK, LNAUCL, and LNAUCINF fell within the acceptance range of 0.6781 to 1.3219 for the investigated dose range. Norgestrel plasma concentrations were also measured to ensure that the norgestrel was being produced proportionally to the concentration of norelgestromin and excess norgestrel was not accumulating over time. Norgestrel PK parameters were also calculated and CPEAK and AUC increased with increased dose administration. In addition, the cumulative mean adhesion score was greater than 95% across all treatments, thus indicating adequate adhesion for all patch sizes when worn for 7 days under normal conditions. Acute dermal irritation was generally mild with similar scores across all three treatments. The residual drug analysis of the worn patches demonstrated that similar relative amounts of norelgestromin were delivered resulting in a proportional daily delivered dose across the 43 mg/7 cm², 3.64/10.5 cm², and 4.86 mg/14 cm² strengths that were investigated.

### Example 10

This example provides various tables comparing the delivery of the contraceptive agent norelgestromin using Unit E accompanied with PK parameters for norgestrel at various wear sites.

Table 10-1 illustrates the delivery of norelgestromin at 4 different application sites of 4.86 mg/14 cm² strength, as illustrated in FIG. 11.

Arithmetic Mean (% CV) Norelgestromin Pharmacokinetic Parameters

| | Abdomen | | Upper Back | | Upper Outer Arm | | Buttock | |
|---|---|---|---|---|---|---|---|---|
| Parameter | (n = 31) | | (n = 31) | | (n = 31) | | (n = 32) | |
| AUCINF (pg*hr/mL) | 174471.3 | (31.5%) | 182134.5 | (38.9%) | 184771.0 | (34.4%) | 156746.9 | (32.2%) |
| AUCL (pg*hr/mL) | 166550.9 | (30.8%) | 174114.2 | (38.8%) | 177212.0 | (33.8%) | 148957.9 | (32.6%) |
| CPEAK (pg/mL) | 1011.674 | (29.4%) | 1090.142 | (39.7%) | 1078.752 | (31.7%) | 922.991 | (33.3%) |
| KEL (1/hr) | 0.0202 | (35.1%) | 0.0193 | (31.7%) | 0.0198 | (30.2%) | 0.0191 | (30.8%) |
| HALFLIFE (hours) | 37.948 | (30.9%) | 39.385 | (31.7%) | 37.658 | (26.2%) | 39.606 | (29.5%) |
| TPEAK (hours) | 97.548 | (30.4%) | 78.194 | (35.4%) | 91.355 | (34.9%) | 105.000 | (32.2%) |
| Daily Dose (mcg/day) | 234 | (21.6%) | 248 | (21.8%) | 221 | (17.1%) | 214 | (19.7%) |

Table 10-2 compares norgestrel to 4 different application sites of 4.86 mg/14 cm² strength.

Arithmetic Mean (% CV) Norgestrel Pharmacokinetic Parameters

| | Abdomen | | Upper Back | | Upper Outer Arm | | Buttock | |
|---|---|---|---|---|---|---|---|---|
| Parameter | (n = 30) | | (n = 30) | | (n = 30) | | (n = 31) | |
| AUCINF (ng*hr/mL) | 62.300 | (58.2%)¹ | 69.791 | (56.0%)² | 77.399 | (86.9%)³ | 56.271 | (64.6%)⁴ |
| AUCL (ng*hr/mL) | 55.904 | (86.8%) | 51.054 | (59.9%) | 60.302 | (100.2%) | 40.607 | (64.1%) |
| CPEAK (ng/mL) | 0.591 | (160.5%) | 0.330 | (58.2%) | 0.657 | (235.8%) | 0.273 | (58.7%) |
| KEL (1/hr) | 0.0129 | (36.6%)¹ | 0.0135 | (40.1%)² | 0.0134 | (47.7%)³ | 0.0121 | (48.9%)⁴ |
| HALFLIFE (hours) | 59.532 | (29.4%)¹ | 62.380 | (51.9%)² | 67.844 | (80.4%)³ | 79.679 | (94.7%)⁴ |

| | | | | | | | |
|---|---|---|---|---|---|---|---|
| TPEAK (hours) | 145.793 | (34.1%) | 132.400 | (22.9%) | 130.533 | (24.6%) | 140.5161.4%) |

¹n = 22,
²n = 26,
³n = 28,
⁴n = 29

Table 10-3 illustrates the summary of patch adhesion.

Summary of Patch Adhesion

| | Abdomen | Upper Back | Upper Outer | Buttock |
|---|---|---|---|---|
| Application Site | (n = 31) | (n = 31) | Arm (n = 31) | (n = 32) |
| Cumulative Mean Score (% CV) | 98.41 (2.07%) | 97.78 (3.42%) | 98.56 (1.81%) | 98.09 (2.69%) |
| Number of Patches > 75% Adhered | 31 | 30 | 31 | 32 |
| Detachments Reported | 0 | 0 | 0 | 0 |

This study demonstrates that the rate and extent of norelgestromin absorption was comparable across the four application sites that were investigated. In addition, the adhesion data revealed a well adhering product with cumulative mean adhesion scores across all application sites being greater than 97% adhered and 124 out of 125 total patches worn in the study, which had greater than a 75% adhesion score across all evaluations over the 7-day wear period. Acute dermal irritation was generally mild with similar scores across all four application sites. The residual drug analysis of the worn patches demonstrated that similar amounts of norelgestromin were delivered across the four application sites that were investigated.

### Example 11

This example provides various tables comparing the delivery of the contraceptive agent norelgestromin using Unit E at various wear sites.

Table 11-1 illustrates the delivery of norelgestromin at 4 different application sites of 2.43 mg/7 cm² strength, as illustrated in FIG. 12.

Arithmetic Mean (% CV) Norelgestromin Pharmacokinetic Parameters

| | Abdomen | | Upper Back | | Upper Outer Arm | | Buttock | |
|---|---|---|---|---|---|---|---|---|
| Parameter | (n = 30) | | (n = 29) | | (n = 31) | | (n = 31) | |
| AUCINF (pg*hr/mL) | 94768.2 | (39.2%) | 102742.1 | (39.6%) | 90809.6 | (35.0%) | 88296.1 | (33.4%) |
| AUCL (pg*hr/mL) | 89701.2 | (39.7%) | 97866.6 | (40.4%) | 86300.3 | (35.5%) | 83097.8 | (31.8%) |
| CPEAK (pg/mL) | 539.430 | (38.2%) | 601.359 | (44.7%) | 525.365 | (33.7%) | 501.374 | (31.1%) |
| KEL (1/hr) | 0.0192 | (35.4%) | 0.0188 | (28.3%) | 0.0191 | (32.3%) | 0.0192 | (31.9%) |
| HALFLIFE (hours) | 40.391 | (33.8%) | 39.846 | (29.3%) | 39.777 | (28.5%) | 40.091 | (36.6%) |
| TPEAK (hours) | 87.200 | (28.4%) | 86.069 | (36.1%) | 94.452 | (33.4%) | 107.613 | (29.9%) |
| Daily Dose (mcg/day) | 109 | (28.8%) | 129 | (26.8%) | 103 | (24.7%) | 106 | (17.8%) |

Table 11-2 illustrates the PK parameters of norgestrel at 4 different application sites of 2.43 mg/7 cm² strength.

Arithmetic Mean (% CV) Norgestrel Pharmacokinetic Parameters

| | Abdomen | | Upper Back | | Upper Outer Arm | | Buttock | |
|---|---|---|---|---|---|---|---|---|
| Parameter | (n = 27) | | (n = 26) | | (n = 30) | | (n = 27) | |
| AUCINF (ng*hr/mL) | 40.420 | (47.9%)¹ | 42.517 | (42.1%)² | 40.061 | (36.3%)³ | 37.381 | (49.4%)¹ |
| AUCL (ng*hr/mL) | 19.685 | (89.7%) | 22.340 | (92.9%) | 19.040 | (92.8%) | 18.124 | (102.7%) |
| CPEAK (ng/mL) | 0.145 | (68.7%) | 0.151 | (82.0%) | 0.135 | (77.7%) | 0.139 | (83.0%) |
| KEL (1/hr) | 0.0118 | (47.7%)¹ | 0.0114 | (47.6%)² | 0.0137 | (52.8%)³ | 0.0120 | (36.8%)¹ |
| HALFLIFE (hours) | 70.483 | (43.0%)¹ | 73.878 | (44.3%)² | 60.600 | (37.0%)³ | 65.188 | (34.0%)¹ |
| TPEAK (hours) | 137.217 | (23.7%) | 133.800 | (26.1%) | 134.261 | (28.5%) | 146.261 | (20.1%) |

¹n = 14,
²n = 17,
³n = 15,
⁴n = 28

Table 11-3 illustrates the summary of patch adhesion.

Summary of Patch Adhesion

| | Abdomen | Upper Back | Upper Outer | Buttock |
|---|---|---|---|---|
| Application Site | (n = 32) | (n = 35) | Arm (n = 33) | (n = 32) |
| Cumulative Mean Score (% CV) | 98.92 (1.46%) | 94.65 (18.7%) | 98.87 (1.73%) | 96.34 (12.95%) |
| Number of Patches > 75% Adhered | 32 | 33 | 33 | 31 |
| Detachments Reported | 0 | 1 | 0 | 1 |

This study demonstrates the rate and extent of norelgestromin absorption across the 4 application sites that were investigated. The concentration of the metabolite norgestrel was comparable between the lower abdomen, upper outer arm, and buttock, but was higher for the upper back application site compared to the other application sites following a single, transdermal patch application worn for 7 days. In addition, the adhesion data revealed a well-adhering product with cumulative mean adhesion scores across all application sites being greater than 94% adhered. In addition, 129 out of 132 total patches worn in the study had greater than a 75% adhesion score across all evaluations over the 7-day wear period. Acute dermal irritation was generally mild with similar scores across all four application sites. The residual drug analysis of the worn patches demonstrated that similar amounts of norelgestromin were delivered across the four application sites that were investigated.

### Example 12

This example provides various tables comparing the delivery of the contraceptive agent norelgestromin using Unit E and the commercially marketed product, XULANE® (Unit A), as illustrated in FIG. 13.

Table 12-1 illustrates the delivery of norelgestromin in both Unit E and Unit A.

Arithmetic Mean (% CV) Norelgestromin
Pharmacokinetic Parameters

| | | | XULANE ® | |
|---|---|---|---|---|
| | Unit E | | (n = 31) | |
| Parameter | (n = 31) | | Unit A | |
| AUCINF (pg*hr/mL) | 181448.3 | (40.2%)¹ | 181009.0 | (35.3%) |
| AUCL (pg*hr/mL) | 170256.5 | (44.2%) | 175984.4 | (34.1%) |
| CPEAK (pg/mL) | 1107.048 | (37.6%) | 1152.242 | (27.3%) |
| KEL (1/hr) | 0.0228 | (22.9%)¹ | 0.0238 | (22.8%) |
| HALFLIFE (hours) | 32.203 | (27.0%)¹ | 30.649 | (23.5%) |
| TPEAK (hours) | 98.409 | (39.2%)¹ | 85.979 | (37.3%) |
| Daily Dose (mcg/day) | 195 | (31.2%) | 202 | (21.5%) |

¹n = 30

As shown in Table 12-1, Unit E delivered a higher concentration of norelgestromin over time at about 181 ng*hr/mL, which is about 0.2% higher than Unit A. Unit E delivers a higher peak concentration of 1.11 ng/mL, which is about 4% lower than Unit A.

Table 12-2 illustrates the PK parameters for norgestrel compared to both Unit E and Unit A.

Arithmetic Mean (% CV) Norgestrel Pharmacokinetic Parameters

| | Unit E | | XULANE ® | |
|---|---|---|---|---|
| Parameter | (n = 25) | | (n = 25) Unit A | |
| AUCINF (ng*hr/mL) | 62.302 | (40.2%)¹ | 194.311 | (43.3%)² |
| AUCL (ng*hr/mL) | 47.895 | (55.9%) | 157.091 | (47.2%) |
| CPEAK (ng/mL) | 0.316 | (52.6%) | 1.107 | (45.6%) |
| KEL (1/hr) | 0.0138 | (30.9%)¹ | 0.0156 | (22.1%)² |
| HALFLIFE (hours) | 55.837 | (37.0%)¹ | 46.900 | (27.3%)² |
| TPEAK (hours) | 133.185 | (18.6%) | 167.695 | (15.3%) |

¹n = 22,
²n = 24

Table 12-3 illustrates the summary of patch adhesion.

Summary of Patch Adhesion

| | Unit E | XULANE ® |
|---|---|---|
| Application Site | (n = 32) | (n = 31) |
| Cumulative Mean Score (% CV) | 97.96 (4.42%) | 99.09 (1.94%) |
| Number of Patches > 75% Adhered | 32 | 31 |
| Detachments Reported | 0 | 0 |

This study demonstrates that the rate and extent of norelgestromin absorption for Unit E's Norelgestromin Transdermal System, 4.86 mg/14 cm² compared to XULANE® Transdermal System (Norelgestromin, 4.86 mg and Ethinyl Estradiol, 0.53 mg/14 cm²) following a single, transdermal patch application on the lower abdomen for 7-days worn under normal conditions. The bioavailability of the metabolite, norgestrel, which was quantified for informational purposes, was much higher following application of XULANE® compared to Unit E's Norelgestromin Transdermal System. In addition, the adhesion data revealed well-adhering products with cumulative mean adhesion scores for both treatments being greater than 97% adhered and all patches worn in the study had greater than a 75% adhesion score across all evaluations over the 7-day wear period.

### Example 13

This example provides various tables comparing the delivery of the contraceptive agent norelgestromin using Unit E and compares it to the pK parameters of the metabolites of norgestrel in different BMI groups, as illustrated in FIG. 14.

Table 13-1 illustrates the delivery of norelgestromin at 3 different BMI groups with 4.86 mg/14 cm² strength.

Arithmetic Mean (% CV) Norelgestromin Pharmacokinetic Parameters

| | BMI 19-25 | | BMI 35-40 | | BMI > 40 | |
|---|---|---|---|---|---|---|
| Parameter | (n = 18) | | (n = 19) | | (n = 15) | |
| AUCINF (pg*hr/mL) | 192258.8 | (20.1%) | 151024.1 | (26.2%) | 153908.2 | (31.1%) |
| AUCL (pg*hr/mL) | 188853.0 | (20.2%) | 139322.9 | (26.6%) | 135673.9 | (32.6%) |
| CPEAK (pg/mL) | 1283.0 | (35.4%) | 839.4 | (24.2%) | 783.2 | (31.8%) |
| KEL (1/hr) | 0.0236 | (13.5%) | 0.0150 | (14.6%) | 0.0124 | (18.1%) |
| HALFLIFE (hours) | 29.92 | (13.1%) | 47.35 | (18.1%) | 57.22 | (16.0%) |
| TPEAK (hours) | 82.67 | (31.8%) | 125.05 | (24.4%) | 139.31 | (18.6%) |
| Daily Dose (mcg/day) | 202 | (24.0%) | 186 | (22.9%) | 185 | (21.4%) |

Table 13-2 illustrates the PK parameters of the metabolite of norgestrel at 3 different BMI groups with 4.86 mg/14 cm² strength.

Arithmetic Mean (% CV) Norgestrel Pharmacokinetic Parameters

| | BMI 19-25 | BMI 35-40 | | BMI > 40 | |
|---|---|---|---|---|---|
| Parameter | (n = 18) | (n = 18) | | (n = 15) | |
| AUCINF | 78.320 (57.7%) | 42.470 | (32.8%)^{a} | 63.313 | (50.6%)^{b} |
| (ng*hr/mL) | | | | | |
| AUCL | 71.164 (59.2%) | 26.587 | (42.5%) | 23.023 | (103.5%) |
| (ng*hr/mL) | | | | | |
| CPEAK | 0.493 (74.0%) | 0.182 | (28.9%) | 0.169 | (78.3%) |
| (ng/mL) | | | | | |
| KEL (1/hr) | 0.0167 (15.1%) | 0.0088 | (31.3%)^{a} | 0.0062 | (43.5%)^{b} |
| HALFLIFE | 42.46 (14.6%) | 89.76 | (45.6%)^{a} | 150.88 | (81.2%)^{b} |
| (hours) | | | | | |
| TPEAK | 121.64 (21.2%) | 143.43 | (19.1%) | 170.29 | (4.94%)^{a} |
| (hours) | | | | | |

^{a}n = 14,
^{b}n = 19

Table 13-3 illustrates the summary of patch adhesion.

Summary of Patch Adhesion

| | BMI 19-25 | BMI 35-40 | BMI > 40 |
|---|---|---|---|
| Application Site | (n = 20) | (n = 20) | (n = 16) |
| Cumulative Mean | 97.38 (6.67%) | 99.57 (1.14%) | 99.26 (1.13%) |
| Score (% CV) | | | |
| Number of Patches > | 19 | 20 | 16 |
| 75% Adhered | | | |
| Detachments Reported | 1 | 0 | 0 |

### Example 14

This example is a Single-Dose Comparative Bioavailability Study of Treatment A, which is norelgestromin 4.86 mg/ethinyl estradiol 0.264 mg transdermal system, Treatment B, which is norelgestromin 4.86 mg/ethinyl estradiol 0.40 mg transdermal system and Treatment C, which is XULANE® Transdermal System (norelgestromin 4.86 mg/ethinyl estradiol 0.53 mg) following a 7-Day wear period in healthy adult female volunteers, as illustrated in FIG. 16. Adhesion and acute irritation data were also provided.

Table 14-1 shows the delivery of norelgestromin accompanied with different concentrations of ethinyl estradiol.

Arithmetic Mean (% CV) Norelgestromin Pharmacokinetic Parameters in Healthy
Adult Female Subjects Following a Single Transdermal Application of Norelgestromin
and Ethinyl Estradiol Transdermal System Worn for 7 Days

| | Trt A | | Trt B | | Trt C = Xulane ® | |
|---|---|---|---|---|---|---|
| | Lot No. 4000752 | | Lot No. 4000710 | | Lot No. 3093154 | |
| Parameter | (n = 24) | | (n = 24) | | (n = 20) | |
| AUCINF (pg*hr/mL) | 161928.6 | (31.0%)¹ | 166150.0 | (28.2%)¹ | 164153.1 | (25.3%) |
| AUCL (pg*hr/mL) | 166897.2 | (39.9%) | 166053.6 | (30.1%) | 160177.4 | (25.8%) |
| CPEAK (pg/mL) | 1133.7 | (35.0%) | 1117.6 | (30.5%) | 1113.1 | (27.5%) |
| KEL (1/hr) | 0.0250 | (20.1%)¹ | 0.0258 | (19.4%)¹ | 0.0264 | (22.7%) |
| HALFLIFE (hours) | 28.73 | (19.4%)¹ | 28.02 | (22.9%)¹ | 27.66 | (24.0%) |
| TPEAK (hours) | 71.06 | (32.4%) | 77.04 | (26.1%) | 74.41 | (44.4%) |

¹n = 23

Table 14-2 shows the delivery of in 3 different concentrations of ethinyl estradiol.

Arithmetic Mean (% CV) Ethinyl Estradiol Pharmacokinetic Parameters in Healthy
Adult Female Subjects Following a Single Transdermal Application of Norelgestromin
and Ethinyl Estradiol Transdermal System Worn for 7 Days

| | Trt A = Mylan | | Trt B = Mylan | | Trt C = Xulane ® |
|---|---|---|---|---|---|
| | Lot No. 4000752 | | Lot No. 4000710 | | Lot No. 3093154 |
| Parameter | (n = 24) | | (n = 22) | | (n = 21) |
| AUCINF (pg*hr/mL) | 6333.5 | (37.6%)¹ | 9375.7 | (47.9%)² | 12466.8 (28.4%) |
| AUCL (pg*hr/mL) | 6602.7 | (44.3%) | 9638.5 | (48.6%) | 12320.2 (28.2%) |
| CPEAK (pg/mL) | 49.88 | (35.4%) | 73.66 | (47.5%) | 98.14 (30.1%) |
| KEL (1/hr) | 0.0411 | (23.3%)¹ | 0.0424 | (30.3%)² | 0.0420 (22.7%) |
| HALFLIFE (hours) | 17.65 | (21.8%)¹ | 18.27 | (41.4%)² | 17.37 (23.9%) |
| TPEAK (hours) | 100.0 | (47.3%) | 89.49 | (39.9%) | 104.1 (42.7%) |

¹n = 23;
²n = 21

Table 14-3 shows FDA 0-4 scale for adhesion of a topical delivery system (TDS)

Scale for Scoring the Comparative Assessment of Adhesion

| Score | Range of Adhered Surface Area for the TDS |
|---|---|
| 0 | ≥90% adhered (i.e., the TDS has essentially no lift off the skin) |
| 1 | ≥75% to <90% adhered (e.g., only some edges of the TDS lift off |
| | the skin) |
| 2 | ≥50% to <75% adhered (i.e., less than half of the TDS lifts off |
| | the skin) |
| 3 | >0% to <50% adhered (i.e., the TDS is not detached, but more than |
| | half of it lifts off the skin without falling off) |
| 4 | 0% adhered (i.e., the TDS is detached and is completely off the |
| | skin) |

Table 14-4 shows the adhesion results in FDA 0-4 scale.

Arithmetic Mean (% CV) of Adhesion Scores Observed in Healthy
Adult Female Subjects Following a Single Dose of Norelgestromin
and Ethinyl Estradiol Transdermal System Worn for 7 Days

| | Trt A | Trt B | Trt C = |
|---|---|---|---|
| | Lot No. | Lot No. | Xulane ® |
| | 4000752 | 4000710 | Lot No. 3093154 |
| Hour | (n = 24) | (n = 24) | (n = 21) |
| 24 | 0.17 (288.9%) | 0.08 (338.8%) | 0.05 (458.3%) |
| 48 | 0.71 (113.9%) | 0.50 (144.5%) | 0.38 (175.6%) |
| 72 | 1.04 (66.3%) | 0.79 (98.4%) | 1.05 (63.9%) |
| 96 | 1.42 (41.2%) | 1.21 (54.5%) | 1.48 (40.8%) |
| 120 | 1.58 (36.9%) | 1.58 (45.3%) | 1.81 (45.0%) |
| 144 | 1.67 (33.9%) | 1.75 (34.7%) | 2.10 (33.4%) |
| 168 | 1.83 (34.8%) | 1.88 (32.7%) | 2.19 (31.0%) |
| Cumulative Mean | 1.20 (41.0%) | 1.11 (44.0%) | 1.29 (38.7%) |

Table 14-5 shows the irritation results.

Frequency of Acute Irritation Scores Observed In Healthy Adult
Female Subjects Following a Single Dose of Norelgestromin
and Ethinyl Estradiol Transdermal System Worn for 7 Days

| | Score = Sum of Dermal Response + | | | | | |
|---|---|---|---|---|---|---|
| | Other Effects | | | | | |
| Treatment | 0 | 1 | 2 | 3 | 6 | Total |
| A = Lot No. 4000752 | 3 | 9 | 7 | 4 | 1 | 24 |
| B = Lot No. 4000710 | 10 | 5 | 7 | 2 | 0 | 24 |
| C = Xulane ® Lot No. 3093154 | 6 | 9 | 2 | 4 | 0 | 21 |
| Total | 19 | 23 | 16 | 10 | 1 | 69 |

This study demonstrates that Treatment A and Treatment B were similar to Treatment C with respect to norelgestromin exposure, and that the rate and extent of exposure for ethinyl estradiol following Treatment A and Treatment B was approximately 50% and 25% lower, respectively, when compared to the reference product, Treatment C, following a single, transdermal patch application worn for 7 days, which is in-line with the design of those treatments relative to Xulane®.

### Example 15

This example provides various tables comparing the delivery of the contraceptive agents norelgestromin and ethinyl estradiol using Unit E patch at different sites and is compared to PK parameters for a commercially marketed oral product, Tri-Lo-Marzia. This example provides pharmacokinetic information for the transdermal system described in this application. The PK data was obtained from 23 healthy women. The subjects applied to skin a single patch application of Treatment A (Norelgestromin 4.86 mg/Ethinyl Estradiol 0.264 mg Transdermal System-applied to an abdomen site), Treatment B having the same strength applied on a buttock site, in both Treatments A and B, and 3 patches were applied over 21 days. Treatment C is a commercially marketed oral product Tri-Lo-Marzia. The pharmacokinetic results are summarized below and are illustrated in FIG. 17 and provide information on the patch compared to oral tablets.

Table 15-1 illustrates the delivery of norelgestromin of Unit E patches applied at Abdomen and buttock sites and PK data for a commercially marketed oral product, Tri-Lo-Marzia.

Arithmetic Mean (% CV) Norelgestromin Pharmacokinetic Parameters

| | Trt A = Unit E | | Trt B = Unit E | | Trt C = Tri-Lo-Marzia ™ | |
|---|---|---|---|---|---|---|
| | Lot No. 4000752 | | Lot No. 4000752 | | Lot No. L801129 | |
| | Abdomen | | Buttocks | | Oral | |
| | Patch 3 (Day 15 to 22) | | Patch 3 (Day 15 to 22) | | Day 7 | |
| Parameter | (n = 23) | | (n = 23) | | (n = 23) | |
| AUCtau (pg*hr/mL) | 132754.4 | (38.4%) | 155284.0 | (31.7%) | 14428.0 | (47.0%) |
| CPEAK (pg/mL) | 1033.5 | (34.0%) | 1191.9 | (24.2%) | 1493.3 | (29.7%) |
| CMIN (pg/mL) | 464.9 | (67.8%) | 509.0 | (55.5%) | 392.9 | (68.5%) |
| TPEAK (hr) | 63.39 | (29.7%)^{a} | 62.83 | (53.7%) | 1.428 | (48.8%) |

Geometric LSMeans Ratios (90% Confidence Intervals) of the natural
log-transformed PK parameters for each test/reference comparison.

| | Treatment | Treatment | Treatment |
|---|---|---|---|
| Parameter | A/Treatment B* | A/Treatment C* | B/Treatment C* |
| LNAUCtau (pg*hr/mL)^{b} | 0.89 | 1.35 | 1.52 |
| | (76.61%-103.25%) | (113.31%-160.12%) | (127.87%-179.67%) |
| LNCPEAK (pg*hr/mL) | 0.90 | 0.72 | 0.80 |
| | (81.91%-99.34%) | (63.77%-81.40%) | (71.29%-90.63%) |
| LNCMIN (pg/mL) | 0.96 | 1.22 | 1.28 |
| | (62.85%-147.44%) | (85.56%-175.35%) | (90.28%-182.59%) |

*Ratio (Test/Reference) = e^{[LSMEAN of (LNTest-LNReference)]}
^{a}n = 22, Subject 11 had no NGMN plasma levels for Patch 3, Treatment A
^{b}The LNAUCtau comparison for A/C and B/C was made by multiplying the Day 7 0-24 hr AUCtau times 7 for Treatment C, to compare with AUCtau 0-168 hr for Treatment A and B.

Table 15-2 illustrates the delivery of ethinyl estradiol of Unit E patches applied at Abdomen and buttock sites and a commercially marketed product, Tri-Lo-Marzia,

Arithmetic Mean (% CV) Ethinyl Estradiol Pharmacokinetic Parameters

| | Trt A = Unit E | Trt B = Unit E | Trt C = Tri-Lo-Marzia ™ |
|---|---|---|---|
| | Lot No. 4000752 | Lot No. 4000752 | Lot No. L801129 |
| | Abdomen | Buttocks | Oral |
| | Patch 3 (Day 15 to 22) | Patch 3 (Day 15 to 22) | Day 7 |
| Parameter | (n = 23) | (n = 23) | (n = 23) |
| AUCtau (pg*hr/mL) | 4695.6 (49.3%) | 5522.2 (35.4%) | 991.0 (51.2%) |
| CPEAK (pg/mL) | 38.06 (43.9%) | 43.46 (27.0%) | 94.02 (35.2%) |
| CMIN (pg/mL) | 13.88 (80.4%) | 16.65 (71.9%) | 23.17 (88.2%) |
| TPEAK (hr) | 80.79 (43.4%)^{a} | 75.89 (47.5%) | 1.449 (36.1%) |

Geometric LSMeans Ratios (90% Confidence Intervals) of the natural
log-transformed PK parameters for each test/reference comparison.

| | Treatment | Treatment | Treatment |
|---|---|---|---|
| Parameter | A/Treatment B* | A/Treatment C* | B/Treatment C* |
| LNAUCtau (pg*hr/mL) | 0.86 | 0.69 | 0.79 |
| | (69.12%-106.00%) | (56.30%-83.50%) | (65.16%-96.02%) |
| LNCPEAK (pg*hr/mL) | 0.90 | 0.42 | 0.47 |
| | (79.06%-101.87%) | (37.43%-48.00%) | (41.46%-52.94%) |
| LNCMIN (pg/mL) | 0.72 | 0.88 | 1.09 |
| | (52.57%-98.23%) | (70.27%-109.69%) | (87.59%-136.73%) |

*Ratio (Test/Reference) = e^{[LSMEAN of (LNTest-LNReference)]}
^{a}n = 22, Subject 11 had no EE plasma levels for Patch 3, Treatment A
^{b}The LNAUCtau comparison for A/C and B/C was made by multiplying the Day 7 0-24 hr AUCtau times 7 for Treatment C, to compare with AUCtau 0-168 hr for Treatment A and B.

Table 15-3 illustrates the summary of patch adhesion.

Arithmetic Mean (% CV) of Adhesion Scores (0-4 FDA Scale)

| | | Trt A = Unit E Lot | Trt B = Unit E |
|---|---|---|---|
| | | No. 4000752 | Lot No. 4000752 |
| | | Abdomen | Buttock |
| Patch No. | Hour | (n = 23) | (n = 23) |
| 1 | 24 | 0.04 (479.6%) | 0.09 (331.3%) |
| | 48 | 0.17 (222.8%) | 0.39 (167.7%) |
| | 72 | 0.65 (99.3%) | 1.22 (78.2%) |
| | 96 | 0.91 (65.3%) | 1.35 (65.6%) |
| | 120 | 1.22 (74.1%) | 1.43 (62.4%) |
| | 144 | 1.43 (58.8%) | 1.74 (46.6%) |
| | 168 | 1.91 (49.6%) | 1.91 (38.3%) |
| 2 | 192 | 0.48 (226.2%) | 0.22 (194.0%) |
| | 216 | 0.83 (144.4%) | 0.52 (97.9%) |
| | 240 | 1.43 (93.6%) | 1.26 (80.1%) |
| | 264 | 1.74 (76.0%) | 1.52 (62.3%) |
| | 288 | 1.87 (65.1%) | 1.70 (54.6%) |
| | 312 | 2.04 (54.2%) | 1.96 (42.1%) |
| | 336 | 2.17 (47.3%) | 2.00 (42.6%) |
| 3 | 360 | 0.17 (222.8%) | 0.26 (172.1%) |
| | 384 | 0.52 (172.1%) | 0.52 (181.6%) |
| | 408 | 0.91 (118.7%) | 0.74 (142.6%) |
| | 432 | 1.22 (95.8%) | 1.39 (64.1%) |
| | 456 | 1.61 (78.9%) | 1.78 (58.5%) |
| | 480 | 1.74 (69.8%) | 1.83 (56.4%) |
| | 504 | 1.96 (56.6%) | 2.13 (40.8%) |

| | | |
|---|---|---|
| Cumulative Mean | 1.19 (51.9%) | 1.24 (36.1%) |

This study demonstrates that Unit E norelgestromin (NGMN) 4.86 mg/ethinyl estradiol 0.264 mg topical delivery system (TDS) delivered lower but comparable amounts of norelgestromin and ethinyl estradiol when worn on the abdomen compared to when worn on the buttocks. In comparison to an orally administered Tri-Lo-Marzia™ containing 0.18 mg norgestimate/0.025 mg ethinyl estradiol at steady-state, Unit E norelgestromin 4.86 mg/Ethinyl Estradiol 0.264 mg TDS demonstrated higher AUCtau and CMIN and lower CPEAK for NGMIN over the 168-hour wear period compared to the oral tablet containing 0.18 mg of norgestimate. For ethinyl estradiol, Unit E Norelgestromin 4.86 mg/Ethinyl Estradiol 0.264 mg TDS demonstrated a lower AUCtau and CPEAK for the abdomen and buttocks with a lower CMIN for the abdomen and a higher CMIN for the buttocks over the 168-hour wear period compared to the oral tablet containing 0.025 mg of ethinyl estradiol. Residual patch analysis revealed that approximately 200 mcg of norelgestromin and approximately 19 mcg of ethinyl estradiol were delivered from each patch per day.

### Example 16

This example provides a summary of a study of the efficacy, safety, tolerability, and adhesion scoring of a low dose estrogen patch (e.g., 0.264 mg ethinyl estradiol) having the adhesive system of Treatment E of Table 1 of the current application in human females.

The study's primary efficacy endpoint was measured by the Pearl Index in women aged 16 to 35 with a body mass index (BMI)<30 kg/m² and where at least one efficacy evaluable cycle was achieved. The Pearl Index is a measure of contraceptive effectiveness, specifically, the number of pregnancies per 100 women-years of contraceptive use. A lower Pearl Index generally indicates a more effective contraceptive method. The Pearl index assumes perfect usage of the contraceptive method and provides a standardized way to compare the effectiveness of different contraceptive methods, with lower numbers generally indicating better protection against pregnancy. In this study, the Pearl Index was 4.14, with 95% Confidence Interval of 2.77 to 5.95. The PI of 4.14 is under 5, which is acceptable for approval of the finished drug product by the FDA. This was surprising for a low dose estrogen patch (e.g., about 17.5 mcg ethinyl estradiol delivered per day). Additionally, the cumulative probability of pregnancy over 13 cycles was 3.7%.

The low dose estrogen patch (e.g., 0.264 mg ethinyl estradiol) having the adhesive system of Treatment E of Table 1 of the current application had a favorable safety and tolerability profile observed with most treatment emergent adverse events (TEAEs) reported as mild-to-moderate. Unscheduled bleeding/spotting decreased between Cycle 1 (34.5%) to Cycle 13 (20%). This indicates that the lower dose of estrogen delivered, about 17.5 mcg ethinyl estradiol per day, is safer as it reduced incidence of adverse bleeding events compared to those bleeding events reported in patches that deliver higher amounts of estrogen such as, for example, Twirla®. In the Cycle Control Population (n=1267), mean number of unscheduled bleeding and/or spotting days ranged between 2.4 to 3.2 days, unscheduled bleeding-only days ranged between 2.1 to 2.6 days, and unscheduled spotting-only days ranged between 0.4 to 0.9 days over 13 cycles. The mean (SD) number of unscheduled bleeding and/or spotting days decreased from Cycle 1 (3.2 days) to Cycle 13 (2.4 days).

The study also demonstrated a favorable patch adhesion profile with very few patches (1.3%) completely detaching over the seven-day wearing period and <1% of subjects reporting severe local application site reactions.

More particularly, a Phase 3 study (NCT05139121) evaluated the contraceptive efficacy and safety of investigational XULANE LO low dose weekly dermal patch of 150 mcg norelgestromin (which can deliver about 200 mcg to about 220 mcg of norelgestromin per day) and 17.5 mcg ethinyl estradiol per day in women of childbearing potential that uses the adhesive system of Treatment E of Table 1 of the current application.

The transdermal system was configured to deliver about 17.5 μg to about 20 μg of ethinyl estradiol per day and about 200 μg to about 220 μg of norelgestromin per day to the woman over a seven (7) day wear period. The amount of ethinyl estradiol and norelgestromin delivered to the woman in the transdermal system was determined by residual analysis of the worn transdermal system. For example, after the transdermal system was worn for about seven days, the transdermal system was removed from the woman and subjected to a residual assay to measure the amount of ethinyl estradiol and the amount of norelgestromin remaining in the transdermal system. The amount delivered to the woman during the wear period was determined based on the difference between (i) the initial amount of ethinyl estradiol and norelgestromin present in the transdermal system prior to application and (ii) the residual amount of ethinyl estradiol and norelgestromin remaining in the transdermal system after the wear period.

The multicenter, open-label, single-arm study evaluated healthy, post-menarcheal, pre-menopausal, heterosexually active female subjects of childbearing potential who are at least 16 years of age (N=1,272) for up to 13 28-day cycles (12,591 safety evaluable cycles and 9,105 efficacy evaluable cycles) across 81 investigative sites throughout the U.S., Puerto Rico and Canada. In this study, XULANE LO demonstrated a favorable efficacy and safety profile with no new safety concerns identified as well as a potential best-in-class patch performance profile.

MR-100A-01 Weekly Transdermal Contraceptive System: Safety Insights from a Phase 3 Trial

In the Phase 3 study (NCT05139121), the safety and tolerability of MR-100A-01, which is a non-invasive transdermal contraceptive system of norelgestromin (150 μg/day) and ethinyl estradiol (17.5 μg/day) was reviewed. The Phase 3 study was a multicenter, open-label, single arm study in healthy, post-menarcheal, pre-menopausal, heterosexually active women of childbearing potential ≥16 years of age, who are at risk of pregnancy. Participants used one patch weekly for 3 weeks, followed by 1 week off, per cycle for 13 cycles. The safety endpoints included incidence of adverse events, serious adverse events and application site reactions. Of the 1,320 enrolled participants, 1,272 (96.4%) were included in the safety population, contributing 12,591 safety evaluable cycles. Treatment-emergent adverse events (TEAEs) were reported in 43.2% of participants, with serious TEAEs occurring in 1.0% and severe TEAEs occurring in 1.8% of participants. Most commonly reported systemic TEAEs (2%) were intermenstrual bleeding (3.9%), COVID-19 (3.4%), nausea (3.3%), urinary tract infection (2.6%), upper respiratory tract infection (2.5%), and headache (2.4%). Application site disorder was reported by 13.8% of participants; most of these were mild to moderate, with only 0.6% being severe. Most common application site reactions (2 2%) were irritation (5%), erythema (3.7%), and pruritus (3.7%). Discontinuation due to TEAEs was reported in 8.0% of participants. No fatal TEAEs occurred during the study. MR-100A-01 showed a favorable safety and tolerability profile. Most TEAEs were mild to moderate, with few severe cases. Less than 1% of participants reported severe local site reactions, demonstrating good local tolerability. No significant differences were found across application sites. Application site irritation dropped from 2.0% (cycle 1) to 0.7% (cycle 13). Patch adhesion improved over time, with the percentage of participants showing no detachment rising from 27.7% in Cycle 1 to 59.5% in Cycle 13. The percentage of all patches used showing no detachment at all increased from 49.2% (cycle 1) to 70.5% (cycle 13). Patches with no detachment are assigned an adhesion score of 0. Additionally, very few patches (1.3%) completely detached over the seven-day wearing period. Patches with complete detachment are assigned an adhesion score of 4.

Contraceptive Efficacy of MR-100A-01 in Women of Childbearing Potential: Results from a Phase 3 Study

In the Phase 3 study (NCT05139121), the contraceptive efficacy of MR-100A-01, which is a weekly transdermal system of norelgestromin (150 pg/day) and ethinyl estradiol (17.5 pg/day) was reviewed. MR-100A-01 offers the dual advantage of a minimally invasive, self-applied contraceptive method with a lower dose of estrogen. This study aimed to evaluate the contraceptive efficacy and safety of the MR-100A-01 patch in women of childbearing potential. This Phase 3, multicenter, open-label, single-arm study (NCT05139121) enrolled healthy, post-menarcheal, heterosexually active women of childbearing potential ≥16 years of age, who were at risk of pregnancy. Participants used one patch weekly for 3 weeks, followed by 1 week off, per-cycle for 13 cycles. The primary endpoint was Pearl Index (PI) in the efficacy evaluable (EE) population (age 16-35 years, BMI<30 kg/m²). Of the 1,320 enrolled participants, 983 (74.5%) were included in the EE population, contributing 9105 EE at risk cycles. The PI was 4.14 (95% CI: 2.77-5.95), indicating 4.14 pregnancies per 100 women-years of use. An alternative calculation, based on the number days of exposure instead of the number of evaluable cycles, provided a similar PI of 4.18 (95% CI: 2.80-6.00). The overall cumulative pregnancy rate was 0.037 [0.04 (95% CI: 0.03-0.05)], corresponding to 3.7% probability of pregnancy within 1 year, reinforcing the contraceptive's effectiveness. Subgroup analyses revealed no clinically meaningful differences across BMI, body weight, race, ethnicity, or prior contraceptive use. Treatment-emergent adverse events (TEAEs) were reported in 43.2% of participants; serious and severe adverse events (AEs) occurred in 1.0% and 1.8%, respectively. Application site disorder was reported by 13.8% of participants, with only 0.6% being severe. This study confirmed the effectiveness of MR-100A-01 in preventing pregnancies in women of childbearing potential with a favorable safety and tolerability profile.

### Example 17

### A Comparative Study of Contraceptive Efficacy of a Low-Dose Ethinyl Estradiol/Norelgestromin Transdermal Patch and TWIRLA®

A transdermal patch was evaluated in a Phase III clinical trial (ClinicalTrials.gov Identifier: NCT05139121). The transdermal contraceptive patch according to an embodiment of the present application was configured to deliver about 17.5 to about 20 μg of ethinyl estradiol per day using Unit E or Treatment E adhesive, as described in the present application. The patch further delivered about 200 μg of norelgestromin per day.

Contraceptive efficacy was assessed using Pearl Index analyses calculated for predefined body mass index (BMI) cohorts. Specifically, Pearl Index values were calculated for: (i) women having a BMI of less than 25 kg/m²; (ii) women having a BMI of from at least 25 kg/m² to less than 30 kg/m²; (iii) women under the age of 35 having a BMI of at least 30 kg/m²; and (iv) an overall population.

The results of the Pearl Index efficacy analysis for the transdermal patch of the present application are summarized in Table 17-1 below.

Table 17-1. Pearl Index Efficacy Analysis for a Transdermal Patch Delivering about 17.5-20 μg Ethinyl Estradiol/Day and about 200 μg Norelgestromin/Day

| BMI | | |
|---|---|---|
| (kg/m²) | Pearl Index | Number of Participants |
| <25 | 4.48 | 4936 |
| ≥25 | 3.74 | 4169 |
| to <30 | | |
| ≥30 | 4.86 (~4.9) | 103 |
| Overall | 4.14 | 9105 |

As shown in Table 17-1, the overall Pearl Index for the patch of the present application was calculated using the cohorts of people with BMI<30 and was calculated to be 4.14, indicating effective contraceptive performance across BMI cohorts.

For comparative purposes, contraceptive efficacy data were evaluated for TWIRLA®, a commercially available transdermal contraceptive patch delivering approximately 30 μg/day ethinyl estradiol and approximately 120 μg/day levonorgestrel. The Pearl Index for TWIRLA® was determined in a separate Phase III clinical trial, with results summarized in Table 17-2.

Table 17-2. Pearl Index Efficacy Analysis for TWIRLA®, a Transdermal Patch Delivering about 30 μg Ethinyl Estradiol/Day and about 120 μg Levonorgestrel/Day

| BMI | Pearl | Number of |
|---|---|---|
| (kg/m²) | Index | Participants |
| <25 | 3.5 | 6007 |
| ≥25 | 5.7 | 3881 |
| to <30 | | |
| ≥30 | 8.6 | 5264 |
| Overall | 5.8 | N/A |

As shown in Table 17-2, the overall Pearl Index for TWIRLA® was 5.8, which is higher than the overall Pearl Index observed for the patch of the present application, despite TWIRLA® delivering a higher daily dose of ethinyl estradiol.

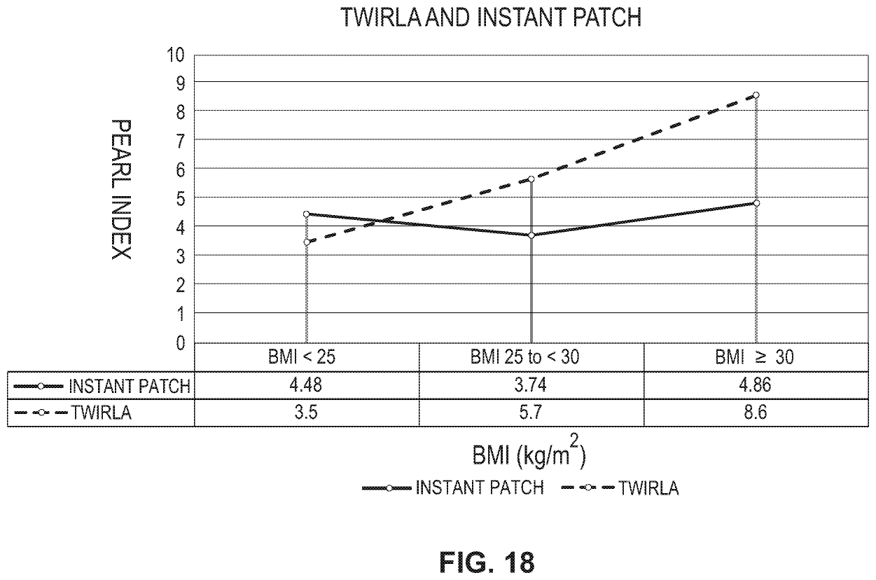

FIG. 18 further shows a graphical comparison of Pearl Index values across BMI cohorts for TWIRLA® and the transdermal patch of the present application, which demonstrates that the Pearl Index for TWIRLA® increases substantially with increasing BMI. In contrast, the low-dose ethinyl estradiol/norelgestromin patch of the present application exhibits a comparatively flatter Pearl Index profile across the BMI cohorts evaluated.

These results were unexpected in view of the lower daily estrogen dose delivered by the patch of the present application relative to TWIRLA®. Additionally, reduced contraceptive efficacy in higher BMI cohorts contributed to a limitation-of-use statement for TWIRLA®, advising consideration of reduced effectiveness in women having a BMI of at least 25 kg/m². By comparison, the patch evaluated in the Phase III trial described herein demonstrated sustained contraceptive effectiveness, as reflected by a consistently low Pearl Index across the BMI categories assessed.

### Example 18

### Updated Adhesion Score Testing

A Phase 3, multicenter, open label, single arm clinical study evaluated the adhesion performance of a once weekly transdermal combined hormonal contraceptive system (MR 100A 01) comprising a reduced dose of ethinyl estradiol (0.264 mg) and norelgestromin (4.86 mg), which was Unit E having the adhesive in Treatment E. Across 1,272 participants and 39,790 applied patches, the transdermal system demonstrated consistently strong adhesion under normal, unrestricted daily conditions. The majority of patches maintained high adhesion (>75%), with low rates of partial detachment and rare complete detachment events. Adhesion performance remained stable over repeated wear cycles, with increasing proportions of fully adherent patches and decreasing rates of partial detachment over time. Performance was comparable across multiple anatomical application sites, including the upper arm, abdomen, buttock, and back, with no meaningful site specific differences observed. Exposure to water, including prolonged exposure, did not significantly impact adhesion outcomes, and investigator assessments corroborated participant reported results, indicating a very low incidence of complete detachment.

More specifically, a total of 39,790 patches were applied by 1,272 participants. Participants reported good adhesion, >75% in 90.5%, partial detachment (50% to <75%) in 4.5%, >50% detachment in 1.0%, and complete detachment in 1.3% of patches.

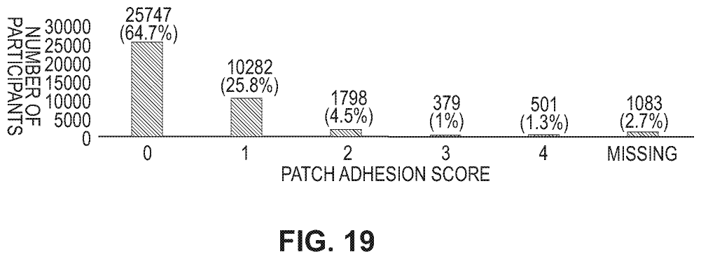

Incidence of complete detachment remained consistently low (0.9% to 1.7% across cycles). A total of 39,790 patches were used during the study, of which 1,872 (4.7%) patches fell off/detached. No notable differences in adhesion score were found across different application sites. Investigator ratings confirmed strong adhesion, with complete detachment observed in only 0.1 to 0.7% of participants. FIG. 19 is a bar graph summarizing adhesion scores for the total of 39,790 patches that were applied by 1,272 participants, which showed improved adherence for Unit E that used the adhesive in Treatment E.

### Conclusion

MR-100A-01 demonstrated good patch adhesion performance with low rates of detachment, with few patches were completely detached over the 7-day wearing period, under unrestricted daily. conditions.

### Embodiments 1-238

1. A transdermal system comprising a backing layer, a release liner, and a matrix disposed between the backing layer and the release liner, the matrix comprising an adhesive, the adhesive comprising a medium molecular weight polyisobutylene having an average molecular weight of 55,000 to 100,000, a high molecular weight polyisobutylene having an average molecular weight of 500,000 to 1,400,000 or both a medium molecular weight polyisobutylene having an average molecular weight of 55,000 to 100,000 and a high molecular weight polyisobutylene having an average molecular weight of 500,000 to 1,500,000.
2. The transdermal system of embodiment 1, wherein the adhesive comprises both a medium molecular weight polyisobutylene having an average molecular weight of 55,000 to 100,000 and a high molecular weight polyisobutylene having an average molecular weight of 500,000 to 1,500,000.
3. The transdermal system of embodiment 1, wherein the adhesive comprises a first component comprising both a medium molecular weight polyisobutylene having an average molecular weight of 55,000 to 100,000 and a high molecular weight polyisobutylene having an average molecular weight of 500,000 to 1,400,000; and the adhesive comprises a second component comprising both a medium molecular weight polyisobutylene having an average molecular weight of 30,000 to 54,000 and a high molecular weight polyisobutylene having an average molecular weight of 1,500,000 to 3,000,000.
4. The transdermal system of embodiment 1, wherein the adhesive comprises a first component comprising both a medium molecular weight polyisobutylene having an average molecular weight of about 70,000 and a high molecular weight polyisobutylene having an average molecular weight of about 1,050,000; and the adhesive comprises a second component comprising both a medium molecular weight polyisobutylene having an average molecular weight of about 53,000 and a high molecular weight polyisobutylene having an average molecular weight of about 1,550,000.
5. The transdermal system of embodiment 3, wherein the weight ratio of the first component of the adhesive to the second component of the adhesive is about 0.5:1 to about 0.5:20.
6. The transdermal system of embodiment 3, wherein the weight ratio of the first component of the adhesive to the second component of the adhesive is about 1:1 to about 1:20.
7. The transdermal system of embodiment 3, wherein the weight ratio of the first component of the adhesive to the second component of the adhesive is about 0.8:10 to about 1:10.
8. The transdermal system of embodiment 3, wherein the weight ratio of the first component of the adhesive to the second component of the adhesive is about 1:8.9 to about 1:9.
9. The transdermal system of embodiment 4, wherein the weight ratio of the first component of the adhesive to the second component of the adhesive is about 0.5:1 to about 0.5:20.
10. The transdermal system of embodiment 4, wherein the weight ratio of the first component of the adhesive to the second component of the adhesive is about 1:1 to about 1:20.
11. The transdermal system of embodiment 4, wherein the weight ratio of the first component of the adhesive to the second component of the adhesive is about 0.8:10 to about 1:10.
12. The transdermal system of embodiment 4, wherein the weight ratio of the first component of the adhesive to the second component of the adhesive is about 1:8.9 to about 1:9.
13. The transdermal system of embodiment 4, wherein the weight ratio of the first component of the adhesive to the second component of the adhesive is about 1:8.9 to about 1:9 such that the transdermal system has an adhesion rate greater than 90% after 7 days of application.
14. The transdermal system of embodiment 3, wherein the adhesive has a medium molecular weight to high molecular weight ratio from about 80:20 to about 90:10 or 80:20 to about 95:5.
15. The transdermal system of embodiment 3, wherein the adhesive has a medium molecular weight to high molecular weight ratio from about 80:20 to about 90:10 such that the transdermal system has an adhesion rate greater than 90% after 7 days of application.
16. The transdermal system of embodiment 3, wherein the adhesive in the first component is in an amount of 1% to 8% by weight based on a total weight of the matrix.
17. The transdermal system of embodiment 3, wherein the adhesive in the first component is in an amount of 3% to 6% by weight based on a total weight of the matrix.
18. The transdermal system of embodiment 3, wherein the adhesive in the first component is in an amount of 5.97% by weight based on a total weight of the matrix.
19. The transdermal system of embodiment 3, wherein the adhesive in the first component is in an amount of 1 mg to 20 mg in a coat weight of 7 to 15 mg/cm².
20. The transdermal system of embodiment 3, wherein the adhesive in the first component is in an amount of 1 mg to 6 mg.
21. The transdermal system of embodiment 3, wherein the adhesive in the first component is in an amount of 12.5 mg.
22. The transdermal system of embodiment 3, wherein the adhesive in the second component is in an amount of 45% to 60% by weight based on a total weight of the matrix.
23. The transdermal system of embodiment 3, wherein the adhesive in the second component is in an amount of 50% to 55% by weight based on a total weight of the matrix.
24. The transdermal system of embodiment 3, wherein the adhesive in the second component is in an amount of 53.54% by weight based on a total weight of the matrix.
25. The transdermal system of embodiment 3, wherein the adhesive in the second component is in an amount of 100 mg to 120 mg in a coat weight of 12.5 to 15 mg/cm².
26. The transdermal system of embodiment 3, wherein the adhesive in the second component is in an amount of 105 mg to 115 mg.
27. The transdermal system of embodiment 3, wherein the adhesive in the second component is in an amount of 112.4 mg.
28. The transdermal system of embodiment 1, wherein the matrix has an adhesion rate of (i) greater than 93%; (ii) greater than 94%; or (iii) greater than 95%.
29. The transdermal system of embodiment 1, wherein the matrix further comprises crospovidone, dipropylene glycol lo+, dipropylene glycol, oleyl alcohol, mineral oil, or a combination thereof.
30. The transdermal system of embodiment 1, wherein the matrix further comprises crospovidone, dipropylene glycol lo+, oleyl alcohol, light mineral oil, or a combination thereof.
31. The transdermal system of embodiment 29, wherein the crospovidone is in an amount of 18% to 30% by weight based on a total weight of the matrix.
32. The transdermal system of embodiment 29, wherein the crospovidone is in an amount of 20% to 30% by weight based on a total weight of the matrix.
33. The transdermal system of embodiment 29, wherein the crospovidone is in an amount of 23.19% by weight based on a total weight of the matrix.
34. The transdermal system of embodiment 29, wherein the crospovidone is in the matrix in an amount of 30 mg to 55 mg in a coat weight of 12.5 to 15 mg/cm².
35. The transdermal system of embodiment 29, wherein the crospovidone is in the matrix in an amount of 40 mg to 50 mg.
36. The transdermal system of embodiment 29, wherein the crospovidone is in the matrix in an amount of 48.7 mg.
37. The transdermal system of embodiment 29, wherein the dipropylene glycol lo+ is in an amount of 0.5% to 1% by weight based on a total weight of the matrix.
38. The transdermal system of embodiment 29, wherein the dipropylene glycol lo+ is in an amount of 0.6% to 6% by weight based on a total weight of the matrix.
39. The transdermal system of embodiment 29, wherein the dipropylene glycol lo+ is in an amount of 0.77% by weight based on a total weight of the matrix.
40. The transdermal system of embodiment 29, wherein the dipropylene glycol lo+ is in the matrix in an amount of 1 mg to 2 mg in a coat weight of 12.5 to 15 mg/cm².
41. The transdermal system of embodiment 29, wherein the dipropylene glycol lo+ is in the matrix in an amount of 1.5 mg to 1.7 mg.
42. The transdermal system of embodiment 29, wherein the dipropylene glycol lo+ is in the matrix in an amount of 1.61 mg.
43. The transdermal system of embodiment 29, wherein the oleyl alcohol is in an amount of 1% to 5% by weight based on a total weight of the matrix.
44. The transdermal system of embodiment 29, wherein the oleyl alcohol is in an amount of 3% to 4% by weight based on a total weight of the matrix.
45. The transdermal system of embodiment 29, wherein the oleyl alcohol is in an amount of 3.64% by weight based on a total weight of the matrix.
46. The transdermal system of embodiment 29, wherein the oleyl alcohol is in the matrix in an amount of 5 mg to 10 mg in a coat weight of 12.5 to 15 mg/cm².
47. The transdermal system of embodiment 29, wherein the oleyl alcohol is in the matrix in an amount of 7 mg to 8 mg.
48. The transdermal system of embodiment 29, wherein the oleyl alcohol is in the matrix in an amount of 7.65 mg.
49. The transdermal system of embodiment 29, wherein the mineral oil is light mineral oil, which is in an amount of 10% to 20% by weight based on a total weight of the matrix.
50. The transdermal system of embodiment 29, wherein the mineral oil is light mineral oil, which is in an amount of 12% to 15% by weight based on a total weight of the matrix.
51. The transdermal system of embodiment 29, wherein the mineral oil is light mineral oil, which is in an amount of 12.89% by weight based on a total weight of the matrix.
52. The transdermal system of embodiment 29, wherein the mineral oil is light mineral oil, which is in the matrix in an amount of 20 mg to 40 mg in a coat weight of 12.5 to 15 mg/cm².
53. The transdermal system of embodiment 29, wherein the mineral oil is light mineral oil, which is in the matrix in an amount of 25 mg to 30 mg.
54. The transdermal system of embodiment 29, wherein the mineral oil is light mineral oil, which is in the matrix in an amount of 27.07 mg.
55. The transdermal system of embodiment 4, wherein the adhesive in the first component is in an amount of 12.5 mg, the adhesive in the second component is in an amount of 112.4 mg, and the matrix further comprises crospovidone in an amount of 48.7 mg, dipropylene glycol LO+ in an amount of 1.61 mg, oleyl alcohol in an amount of 7.65 mg and light mineral oil in an amount of 27.07.
56. The transdermal system of embodiment 4, wherein the adhesive in the first component is in an amount of 5.97% by weight, the adhesive in the second component is in an amount of 53.54% by weight based on the total weight of the matrix, and the matrix further comprises crospovidone in an amount of 23.19% by weight, dipropylene glycol LO+ in an amount of 0.77% by weight, oleyl alcohol in an amount of 3.64% by weight and light mineral oil in an amount of 12.89% by weight based on the total weight of the matrix.
57. The transdermal system of embodiment 55, wherein the transdermal system has a size of 14 cm² and the transdermal system has a total matrix basis weight of 150 g/m².
58. The transdermal system of embodiment 56, wherein the transdermal system has a size of 14 cm² and the transdermal system has a total matrix basis weight of 150 g/m².
59. The transdermal system of embodiment 55, wherein the matrix has a coat weight of 12.5 to 15 mg/cm².
60. The transdermal system of embodiment 56, wherein the matrix has a coat weight of 12.5 to 15 mg/cm².
61. The transdermal system of embodiment 1, wherein the backing has a thickness from about 1 to about 2 millimeters.
62. The transdermal system of embodiment 1, wherein the backing has a thickness of about 1.5 millimeters.
63. The transdermal system of embodiment 1, wherein the matrix in addition to the adhesive comprises at least one active pharmaceutical ingredient.
64. The transdermal system of embodiment 63, wherein the active pharmaceutical ingredient comprises an estrogen, a progestin or a combination of an estrogen and a progestin.
65. The transdermal system of embodiment 64, wherein the progestin comprises norelgestromin and the estrogen comprises ethinyl estradiol.
66. The transdermal system of embodiment 64, wherein the matrix does not contain an estrogen.
67. The transdermal system of embodiment 64, wherein the matrix does not contain a progestin.
68. The transdermal system of embodiment 65, wherein the matrix is configured to release over a period of seven days the norelgestromin to produce an AUC∞ of about 167 ng·hr/mL to about 239 ng·hr/mL, a Cₘₐₓ of about 1.1 ng/mL to about 1.5 ng/mL and a t_{1/2} of about 30 hours to about 37 hours.
69. The transdermal system of embodiment 65, wherein the matrix is configured to release over a period of seven days the ethinyl estradiol to produce an AUC∞ of about 4493 pg·hr/mL to about 6707 pg·hr/mL, a Cₘₐₓ of about 33.8 pg/mL to about 50.1 pg/mL and a t_{1/2} of about 18 hours to about 23 hours.
70. The transdermal system of embodiment 65, wherein the matrix is configured to release over a period of seven days the norelgestromin to produce an AUC∞ of about 217 ng·hr/mL, a Cₘₐₓ of about 1.37 ng/mL and a t_{1/2} of about 32 hours.
71. The transdermal system of embodiment 65, wherein the matrix is configured to release over a period of seven days the ethinyl estradiol to produce an AUC∞ of about 6193 pg·hr/mL, a Cₘₐₓ of about 45.58 pg/mL and a t_{1/2} of about 21 hours.
72. The transdermal system of embodiment 65, wherein the matrix is configured to release over a period of seven days the norelgestromin to produce an AUC∞ of about 150 ng·hr/mL to about 183 ng·hr/mL, a Cₘₐₓ of about 0.93 ng/mL to about 1.13 ng/mL and a t_{1/2} of about 27 hours to about 33 hours.
73. The transdermal system of embodiment 65, wherein the matrix is configured to release over a period of seven days the ethinyl estradiol to produce an AUC∞ of about 3783 pg·hr/mL to about 4624 pg·hr/mL, a Cₘₐₓ of about 25.5 pg/mL to about 31.2 pg/mL and a t_{1/2} of about 22 hours to about 27 hours.
74. The transdermal system of embodiment 65, wherein the matrix is configured to release over a period of seven days the norelgestromin to produce an AUC∞ of about 166 ng·hr/mL, a Cₘₐₓ of about 1.03 ng/mL and a t_{1/2} of about 30 hours.
75. The transdermal system of embodiment 65, wherein the matrix is configured to release over a period of seven days the ethinyl estradiol to produce an AUC∞ of about 4203 pg·hr/mL, a Cₘₐₓ of about 28.4 pg/mL and a t_{1/2} of about 24 hours.
76. The transdermal system of embodiment 65, wherein the matrix is configured to release over a period of seven days the norelgestromin to produce an AUC∞ of about 94 ng·hr/mL to about 219 ng·hr/mL, a Cₘₐₓ of about 0.6 ng/mL to about 1.18 ng/mL and a t_{1/2} of about 38 hours to about 44 hours.
77. The transdermal system of embodiment 65, wherein the matrix is configured to release over a period of seven days the norelgestromin to produce an AUC∞ of about 199 ng·hr/mL, a Cₘₐₓ of about 1.18 ng/mL and a t_{1/2} of about 38 hours.
78. The transdermal system of embodiment 65, wherein the matrix is configured to release over a period of seven days the norelgestromin to produce an AUC∞ of about 185 ng·hr/mL, a Cₘₐₓ of about 1.08 ng/mL and a t_{1/2} of about 38 hours.
79. The transdermal system of embodiment 65, wherein the matrix is configured to release over a period of seven days the norelgestromin to produce an AUC∞ of about 103 ng·hr/mL, a Cₘₐₓ of about 0.53 ng/mL and a t_{1/2} of about 40 hours.
80. The transdermal system of embodiment 65, wherein the matrix is configured to release over a period of seven days the norelgestromin to produce an AUC∞ of about 181 ng·hr/mL, a Cₘₐₓ of about 1.1 ng/mL and a t_{1/2} of about 32 hours.
81. The transdermal system of embodiment 65, wherein the matrix comprises 4.86 mg of norelgestromin.
82. The transdermal system of embodiment 65, wherein the matrix comprises 2-5 mg of norelgestromin in a coat weight of 15 mg/cm².
83. The transdermal system of embodiment 65, wherein the matrix comprises 4.86 mg of norelgestromin in a coat weight of 15 mg/cm².
84. The transdermal system of embodiment 65, wherein the matrix comprises 2.31% by weight of norelgestromin.
85. The transdermal system of embodiment 65, wherein the matrix comprises 2.0-4.0% by weight of norelgestromin in a coat weight of 15 mg/cm².
86. The transdermal system of embodiment 65, wherein the matrix comprises 2.31% by weight of norelgestromin in a coat weight of 15 mg/cm².
87. The transdermal system of embodiment 65, wherein the matrix comprises 4.86 mg of norelgestromin and no ethinyl estradiol per 14 cm² of the matrix.
88. The transdermal system of embodiment 65, wherein the matrix comprises 3.64 mg of norelgestromin and no ethinyl estradiol per 10.5 cm² of the matrix.
89. The transdermal system of embodiment 65, wherein the matrix comprises 2.43 mg of norelgestromin and no ethinyl estradiol per 7 cm² of the matrix.
90. The transdermal system of embodiment 65, wherein the matrix comprises 0.53 mg of ethinyl estradiol.
91. The transdermal system of embodiment 65, wherein the matrix comprises 0.25-1.0 mg of ethinyl estradiol in a coat weight of 15 mg/cm².
92. The transdermal system of embodiment 65, wherein the matrix comprises 0.53 mg of ethinyl estradiol in a coat weight of 15 mg/cm².
93. The transdermal system of embodiment 65, wherein the matrix comprises 0.25% by weight of ethinyl estradiol.
94. The transdermal system of embodiment 65, wherein the matrix comprises 0.1-0.5% by weight of ethinyl estradiol in a coat weight of 15 mg/cm².
95. The transdermal system of embodiment 65, wherein the matrix comprises 0.25% by weight of ethinyl estradiol in a coat weight of 15 mg/cm².
96. The transdermal system of embodiment 1, wherein the matrix is applied to a patient having (i) BMI between 19-25; (ii) BMI between 35-40; or (iii) BMI greater than 40.
97. The transdermal system of any one of embodiments 1-96, wherein the transdermal system can be applied to an arm, an abdomen, a buttock or a back of a human patient.
98. A transdermal system for releasing a contraceptive agent to a patient in need thereof, the transdermal system comprising a backing layer, a release liner, and a matrix disposed between the backing layer and the release liner, the matrix comprising an adhesive, the adhesive comprising a medium molecular weight polyisobutylene having an average molecular weight of 55,000 to 100,000, a high molecular weight polyisobutylene having an average molecular weight of 500,000 to 1,400,000 or both a medium molecular weight polyisobutylene having an average molecular weight of 55,000 to 100,000 and a high molecular weight polyisobutylene having an average molecular weight of 500,000 to 1,500,000, wherein the matrix is configured to release about 100 mcg per day to about 300 mcg per day of the contraceptive agent to the patient.
99. The transdermal system of embodiment 98, wherein the backing is in the form of a layer and is impermeable to the contraceptive agent, and the transdermal system is configured to deliver about 193 mcg per day to about 257 mcg per day of the contraceptive agent progestin to the patient.
100. The transdermal system of embodiment 1, wherein the release liner is configured to be removed from the matrix.
101. The transdermal system of embodiment 1, wherein the transdermal system is in the form of a patch or film.
102. The transdermal system of embodiment 98, wherein the contraceptive agent comprises norelgestromin, which is released from the transdermal system in an amount of about 93 mcg per day to about 142 mcg per day.
103. The transdermal system of embodiment 1, wherein the adhesive is in the matrix in an amount of about 56% w/w to about 60% w/w based on a total % w/w of the matrix.
104. The transdermal system of embodiment 1, wherein the transdermal system is in a patch or film form and the adhesive is in the matrix in an amount of about 123 mg to about 127 mg per patch or film.
105. The transdermal system of embodiment 98, wherein the transdermal system has constant release of the contraceptive agent at about 48 hours to about 168 hours after the transdermal system is applied to a skin of the patient.
106. The transdermal system of embodiment 98, wherein the transdermal system is configured to release over a period of seven days the contraceptive agent norelgestromin to produce an AUC∞ of about 147 ng·hr/mL to about 197 ng·hr/mL, a Cₘₐₓ of about 0.92 ng/mL to about 1.26 ng/mL and a t_{1/2} of about 30 hours to about 33 hours.
107. The transdermal system of embodiment 98, wherein the transdermal system is configured to release over a period of seven days the contraceptive agent ethinyl estradiol to produce an AUC∞ about 3.7 ng·hr/mL to about 62.3 ng·hr/mL, a Cₘₐₓ about 0.27 ng/mL to about 0.45 ng/mL and a t_{1/2} about 20 hours to about 21 hours.
108. The transdermal system of embodiment 98, wherein the transdermal system has a skin contacting surface having a surface area of about 14 cm² or less.
109. The transdermal system of embodiment 98, wherein the transdermal system is configured to release the contraceptive agent norelgestromin in an amount of (i) 193 to 263 mcg/day; (ii) about 169 to about 206 mcg/day; (iii) about 166 to about 202 mcg/day; (iv) about 108 to about 253 mcg/day; (v) about 193 to about 273 mcg/day; or (vi) about 93 to about 142 mcg/day.
110. The transdermal system of embodiment 1, wherein the backing comprises polyvinylchloride, polyvinylidene chloride, ethylene-vinyl acetate copolymers, polyethylene, polypropylene, polyurethane, or polyethylene terephthalate.
111. The transdermal system of embodiment 1, wherein the matrix is disposed on a woven or non-woven polymer fabric.
112. The transdermal system of embodiment 111, wherein the polymer fabric is polyester.
113. The transdermal system of embodiment 112, wherein the polymer fabric is non-woven polyester.
114. The transdermal system of embodiment 1, wherein the matrix further comprises one or more skin permeation enhancers comprising lactate ester of C12 to Cis aliphatic alcohol, lauryl lactate, oleic acid, dipropylene glycol LO+, dipropylene glycol, propylene glycol monolaurate (PGML) or a combination thereof.
115. The transdermal system of embodiment 98, wherein the transdermal system is applied to skin of the patient in a regimen comprising application of one transdermal system once each week for three consecutive weeks.
116. The transdermal system of embodiment 98, wherein the transdermal system is applied to skin of the patient in a regimen comprising application of one transdermal system once each week for three consecutive weeks, followed by one week in which the transdermal system is not applied.
117. The transdermal system of any one of embodiments 98-116, wherein the transdermal system can be applied to an arm, an abdomen, a buttock or a back of a human patient.
118. A method of providing an active pharmaceutical ingredient to a patient in need thereof, the method comprising applying to the skin of the patient a transdermal system of any one of embodiments 1, 2 or 3 so as to release the active pharmaceutical agent through the skin of the patient.
119. The method of embodiment 118, wherein the active pharmaceutical ingredient comprises a contraceptive agent and the transdermal system is configured to release about 100 mcg per day to about 300 mcg per day of the contraceptive agent to the patient.
120. The method of embodiment 119, wherein the contraceptive agent comprises an estrogen, a progestin or a combination thereof.
121. The method of embodiment 120, wherein the backing is in the form of a layer and is impermeable to the estrogen or progestin, and the matrix is configured to contact the skin of the patient and allow the estrogen, progestin or both the estrogen and progestin to be released from the transdermal system and through the skin of the patient.
122. The method of embodiment 118, wherein the transdermal system further comprises a release liner contacting the matrix and configured to be removed from the matrix prior to contacting the skin of the patient.
123. The method of embodiment 121, wherein (i) the transdermal system has a skin contacting surface having a surface area of about 14 cm² or less; or (ii) the transdermal system is configured to deliver about 17 mcg per day to about 35 mcg per day of an estrogen to the patient.
124. The method of embodiment 121, wherein the transdermal system is configured to release estrogen from the transdermal system in an amount of about 19 mcg per day to about 40 mcg per day.
125. The method of embodiment 121, wherein the transdermal system is configured to release progestin in an amount of (i) 106 to 248 mcg/day; (ii) about 214 to about 248 mcg/day; or (iii) about 106 to about 129 mcg/day.
126. The method of embodiment 121, wherein an oral contraceptive is administered together with the transdermal system.
127. The method of embodiment 126, wherein the oral contraceptive is a progestin.
128. The method of embodiment 127, wherein the progestin is norgestrel.
129. The transdermal system of any one of embodiments 116-128, wherein the transdermal system can be applied to an arm, an abdomen, a buttock or a back of a human patient.
130. A method of making a transdermal system comprising a backing layer, a release liner, and a matrix disposed between the backing layer and the release liner, the method comprising contacting the matrix with an adhesive, the adhesive comprising a medium molecular weight polyisobutylene having an average molecular weight of 55,000 to 100,000, a high molecular weight polyisobutylene having an average molecular weight of 500,000 to 1,400,000 or both a medium molecular weight polyisobutylene having an average molecular weight of 55,000 to 100,000 and a high molecular weight polyisobutylene having an average molecular weight of 500,000 to 1,500,000.
131. The method of embodiment 130, wherein the adhesive comprises a first component comprising both a medium molecular weight polyisobutylene having an average molecular weight of 55,000 to 100,000 and a high molecular weight polyisobutylene having an average molecular weight of 500,000 to 1,400,000; and the adhesive comprises a second component comprising both a medium molecular weight polyisobutylene having an average molecular weight of 30,000 to 54,000 and a high molecular weight polyisobutylene having an average molecular weight of 1,500,000 to 3,000,000.
132. The method of embodiment 130, further comprising applying crospovidone, dipropylene glycol lo+, oleyl alcohol, light mineral oil, or a combination thereof to the matrix.
133. The method of embodiment 130, further comprising applying the active pharmaceutical ingredient to the matrix.
134. The method of making the transdermal system of any one of embodiments 1-128, the method comprising applying the adhesive to the matrix so as to form a patch or film.
135. A method of making a transdermal system, the method comprising mixing about 2.2 mg to about 5.3 mg of progestin per 7 to 14 cm² with a matrix, the matrix comprising an adhesive comprising a medium molecular weight polyisobutylene having an average molecular weight of 55,000 to 100,000, a high molecular weight polyisobutylene having an average molecular weight of 500,000 to 1,400,000 or both a medium molecular weight polyisobutylene having an average molecular weight of 55,000 to 100,000 and a high molecular weight polyisobutylene having an average molecular weight of 500,000 to 1,500,000, wherein the transdermal system is configured to release about 108 mcg per day to about 253 mcg per day of the progestin to a patient.
136. The method of embodiment 135, wherein the progestin comprises norelgestromin.
137. The method of embodiment 135, wherein the transdermal system further comprises an estrogen and the estrogen is also mixed with the progestin.
138. The method of embodiment 137, wherein the estrogen comprises ethinyl estradiol and is released from the transdermal system in an amount of about 16 mcg per day to about 23 mcg per day.
139. The method of embodiment 138, wherein the ethinyl estradiol is in the transdermal system in the matrix in an amount of about 0.53 mg in a coat weight of 15 mg/cm².
140. The method of embodiments 130-134, further comprising laminating a backing film to the matrix.
141. The method of any one of embodiments 128-139, wherein the transdermal system can be applied to an arm, an abdomen, a buttock or a back of a human patient.
142. The transdermal system of any one of embodiments 1-97, wherein the transdermal system has a Pearl Index of between 4.0 and less than 5.0.
143. The transdermal system of any one of embodiments 1-97, wherein the transdermal system has a Pearl Index of between 1.0 to less than 5.0.
144. The transdermal system of any one of embodiments 1-97, wherein the transdermal system has a Pearl Index of between 1.5 to less than 4.14.
145. The transdermal system of any one of embodiments 1-97, wherein the transdermal system has a Pearl Index of between 2.7 to less than 5.95.
146. The transdermal system of any one of embodiments 1-97, wherein the transdermal system has unscheduled bleeding/spotting decreased by under 40% when worn on the skin.
147. The transdermal system of any one of embodiments 1-97, wherein the transdermal system has unscheduled bleeding/spotting decreased by under 35% or less when worn on the skin.
148. The transdermal system of any one of embodiments 1-97, wherein the transdermal system has unscheduled bleeding/spotting decreased by about 20% or less when worn on the skin.
149. The transdermal system of any one of embodiments 1-97, wherein the transdermal system has unscheduled bleeding/spotting decreased by about 34.5% in Cycle 1 to about 20% in Cycle 1.
150. The transdermal system of any one of embodiments 1-97, wherein the transdermal system has no or less than 1.3% of patches completely detaching over the seven-day wearing period.
151. The transdermal system of any one of embodiments 1-97, wherein the transdermal system has no or less than <1% of patients reporting severe local application site reactions.
152. The transdermal system of any one of embodiments 1-97, wherein the transdermal system delivers 150 mcg of norelgestromin per day.
153. The transdermal system of any one of embodiments 1-97, wherein the transdermal system delivers 17.5 mcg of ethinyl estradiol per day.
154. The transdermal system of any one of embodiments 1-97, wherein the transdermal system delivers 150 mcg of norelgestromin and 17.5 mcg of ethinyl estradiol per day.
155. The transdermal system of any one of embodiments 1-97 and 152-154, wherein the transdermal system is worn at least seven days by a human female.
156. The transdermal system of any one of embodiments 1-97 and 152-154, wherein the transdermal system has less than 2% of severe adverse reactions when the transdermal system is worn.
157. The transdermal system of any one of embodiments 1-97 and 152-154, wherein the transdermal system has less than 1% of severe local site adverse reactions when the transdermal system is worn.
158. The transdermal system of any one of embodiments 1-97 and 152-154, wherein the transdermal system has a pregnancy rate of 4% or less in 1 year.
159. The transdermal system of any one of embodiments 1-97 and 152-154, wherein the transdermal system has a pregnancy rate of 4.0/6-4.2% or less in 1 year.
160. A transdermal system of any one of embodiments 1-108, and 152-154 that delivers 17.5 mcg of ethinyl estradiol per day.
161. A transdermal system of any one of embodiments 1-108, and 152-154 that delivers 150 mcg of norelgestromin and 17.5 mcg of ethinyl estradiol per day.
162. A transdermal system comprising a backing layer, a release liner, and a matrix disposed between the backing layer and the release liner, the matrix comprising an active pharmaceutical ingredient and an adhesive, the adhesive comprising a first component comprising both a medium molecular weight polyisobutylene having an average molecular weight of 55,000 to 100,000 and a high molecular weight polyisobutylene having an average molecular weight of 500,000 to 3,000,000; and the adhesive comprising a second component comprising both a medium molecular weight polyisobutylene having an average molecular weight of 30,000 to 54,000 and a high molecular weight polyisobutylene having an average molecular weight of 500,000 to 30,000,000; and wherein the first component and the second component has a weight ratio from about 20:80 to about 10:90.
163. The transdermal system of embodiment 162, wherein the first component comprises the medium molecular weight polyisobutylene having an average molecular weight of about 70,000 and the high molecular weight polyisobutylene comprising a first high molecular weight polyisobutylene having an average molecular weight of about 1,050,000; and a second high molecular weight polyisobutylene having an average molecular weight of about 1,550,000; and the second component comprises the medium molecular weight polyisobutylene having an average molecular weight of about 53,000 and the high molecular weight polyisobutylene having an average molecular weight of about 1,550,000.
164. The transdermal system of embodiment 163, wherein the first component comprises 55% of the medium molecular polyisobutylene, 22.5% of the first high molecular weight polyisobutylene and 22.5% of the second high molecular weight polyisobutylene, and the second component comprises 84% of the medium molecular weight polyisobutylene and 16% of the high molecular weight polyisobutylene; and wherein the first component and the second component have a weight ratio of 10:90 such that the first component and the second component combined have a polyisobutylene mixture comprising 75.6% of a first medium molecular weight polyisobutylene, 5.5% of a second medium molecular weight polyisobutylene, 2.25% of a second high molecular weight polyisobutylene, and 16.65% of a first high molecular weight polyisobutylene.
165. The transdermal system of embodiment 162, wherein the transdermal system has an adhesion rate greater than 90% after 7 days of application to a childbearing human female.
166. The transdermal system of embodiment 162, wherein the adhesive in the first component is in an amount of about 12.5 mg, the adhesive in the second component is in an amount of about 112.4 mg, and the matrix further comprises crospovidone in an amount of about 48.7 mg, dipropylene glycol LO+ in an amount of 1.61 mg, oleyl alcohol in an amount of about 7.65 mg and light mineral oil in an amount of about 27.07 mg.
167. The transdermal system of embodiment 162, wherein the active pharmaceutical ingredient comprises an estrogen comprising ethinyl estradiol, a progestin comprising norelgestromin or a combination of an estrogen and a progestin.
168. The transdermal system of embodiment 162, wherein the matrix does not contain an estrogen.
169. The transdermal system of embodiment 162, wherein the matrix does not contain a progestin.
170. The transdermal system of embodiment 162, wherein the transdermal system has a Pearl Index between 1.5 to 4.18.
171. The transdermal system of embodiment 162, wherein the transdermal system delivers 150 mcg of norelgestromin per day and 17.5 mcg of ethinyl estradiol per day to a patient.
172. The transdermal system of embodiment 162, wherein the transdermal system has a pregnancy rate of 4% or less within 1 year.
173. A transdermal system comprising a backing layer, a release liner, and a matrix disposed between the backing layer and the release liner, the matrix comprising norelgestromin, ethinyl estradiol, and an adhesive, the adhesive comprising a first component comprising a medium molecular weight polyisobutylene having an average molecular weight of about 70,000, a first high molecular weight polyisobutylene having an average molecular weight of about 1,050,000 and a second high molecular weight polyisobutylene having an average molecular weight of about 1,550,000; and the adhesive comprises a second component comprising both a medium molecular weight polyisobutylene having an average molecular weight of about 53,000 and a high molecular weight polyisobutylene having an average molecular weight of about 1,550,000, and the first component and the second component have a weight ratio of about 10:90.
174. The transdermal system of embodiment 173, wherein the transdermal system delivers 150 mcg of norelgestromin per day and 17.5 mcg of ethinyl estradiol per day.
175. The transdermal system of embodiment 173, wherein the matrix is configured to release over a period of seven days the norelgestromin to produce an AUC∞ of about 167 ng·hr/mL to about 239 ng·hr/mL, a Cmax of about 1.1 ng/mL to about 1.5 ng/mL and a t½ of about 30 hours to about 37 hours; and release over a period of seven days the ethinyl estradiol to produce an AUC∞ of about 4493 pg·hr/mL to about 6707 pg·hr/mL, a Cmax of about 33.8 pg/mL to about 50.1 pg/mL and a t½ of about 18 hours to about 23 hours.
176. The transdermal system of embodiment 173, wherein the matrix is configured to release over a period of seven days the norelgestromin to produce an AUC∞ of about 217 ng·hr/mL, a Cmax of about 1.37 ng/mL and a t½ of about 32 hours; and release over a period of seven days the ethinyl estradiol to produce an AUC∞ of about 6193 pg·hr/mL, a Cmax of about 45.58 pg/mL and a t½ of about 21 hours.
177. The transdermal system of embodiment 173, wherein the matrix is configured to release over a period of seven days the norelgestromin to produce an AUC∞ of about 150 ng·hr/mL to about 183 ng·hr/mL, a Cmax of about 0.93 ng/mL to about 1.13 ng/mL and a t½ of about 27 hours to about 33 hours; and release over a period of seven days the ethinyl estradiol to produce an AUC∞ of about 3783 pg·hr/mL to about 4624 pg·hr/mL, a Cmax of about 25.5 pg/mL to about 31.2 pg/mL and a t½ of about 22 hours to about 27 hours.
178. The transdermal system of embodiment 173, wherein the transdermal system has an adhesion rate greater than 90% after 7 days of application.
179. The transdermal system of embodiment 173, wherein the transdermal system has an adhesion rate greater than 90% after 7 days of application to a childbearing human female.
180. The transdermal system of embodiment 173, wherein the transdermal system has a Pearl Index of 1.5 to 4.18.
181. A transdermal system comprising a backing layer, a release liner, and a matrix disposed between the backing layer and the release liner, the matrix comprising an active pharmaceutical ingredient comprising norelgestromin and ethinyl estradiol; and an adhesive, the adhesive comprising a first component comprising both a medium molecular weight polyisobutylene having an average molecular weight of about 70,000 and a high molecular weight polyisobutylene having a first high molecular weight polyisobutylene having an average molecular weight of about 1,050,000, and a second high molecular weight polyisobutylene having an average molecular weight of about 1,550,000; and the adhesive comprising a second component comprising both a medium molecular weight polyisobutylene having an average molecular weight of about 53,000 and a high molecular weight polyisobutylene having an average molecular weight of about 1,550,000 so as to release the active pharmaceutical agent through the skin of the patient, wherein the first component comprises about 55% of a first medium molecular weight polyisobutylene of the medium molecular weight polyisobutylene, about 22.5% of a first high molecular weight polyisobutylene of the high molecular weight polyisobutylene and about 22.5% of a second high molecular weight polyisobutylene of the high molecular weight polyisobutylene; and second component comprises about 84% of a second medium molecular weight polyisobutylene of the medium molecular weight polyisobutylene and about 16% of the first high molecular weight polyisobutylene of the high molecular weight polyisobutylene; and wherein the first component and the second component have a weight ratio of about 10:90 such that the first component and the second component combined have a polyisobutylene mixture comprising about 5.5% of the first medium molecular weight polyisobutylene, about 75.6% of the second medium molecular weight polyisobutylene, about 16.65% of the first high molecular weight polyisobutylene; and about 2.25% of the second high molecular weight polyisobutylene.
182. The transdermal system of embodiment 181, wherein (i) the matrix is configured to release over a period of seven days the norelgestromin to produce an AUC∞ of about 167 ng·hr/mL to about 239 ng·hr/mL, a Cmax of about 1.1 ng/mL to about 1.5 ng/mL and a t½ of about 30 hours to about 37 hours; (ii) the matrix is configured to release over a period of seven days the ethinyl estradiol to produce an AUC∞ of about 4493 pg·hr/mL to about 6707 pg·hr/mL, a Cmax of about 33.8 pg/mL to about 50.1 pg/mL and a t½ of about 18 hours to about 23 hours; (iii) the matrix is configured to release over a period of seven days the norelgestromin to produce an AUC∞ of about 217 ng·hr/mL, a Cmax of about 1.37 ng/mL and a t½ of about 32 hours; or (iv) the matrix is configured to release over a period of seven days the ethinyl estradiol to produce an AUC∞ of about 6193 pg·hr/mL, a Cmax of about 45.58 pg/mL and a t½ of about 21 hours.
183. The transdermal system of embodiment 162, wherein the transdermal system delivers about 150 mcg to about 200 mcg of norelgestromin per day.
184. The transdermal system of embodiment 162, wherein the transdermal system delivers about 150 mcg to about 200 mcg of norelgestromin per day as the only active pharmaceutical ingredient.
185. The transdermal system of embodiment 162, wherein the transdermal system delivers about 17.5 mcg of ethinyl estradiol per day to a patient.
186. The transdermal system of embodiment 162, wherein the transdermal system delivers about 17.5 mcg of ethinyl estradiol per day to a patient as the only active pharmaceutical ingredient.
187. The transdermal system of embodiment 181, wherein the transdermal system delivers 150 mcg of norelgestromin per day and 17.5 mcg of ethinyl estradiol per day.
188. The transdermal system of embodiment 181, wherein the transdermal system is for use in a childbearing human female.
189. The transdermal system of embodiment 181, wherein the transdermal system has a Pearl Index between 1.0 to less than 5.0.
190. A transdermal system comprising a backing layer, a release liner, and a matrix disposed between the backing layer and the release liner, the matrix comprising norelgestromin, ethinyl estradiol, and an adhesive, the adhesive comprising a first component comprising 55% of a first medium molecular weight polyisobutylene, 22.5% of a first high molecular weight polyisobutylene, and 22.5% of a second high molecular weight polyisobutylene; and a second component comprising 84% of a second medium molecular weight polyisobutylene and 16% of a first high molecular weight polyisobutylene, and wherein the first component and the second component has a weight ratio of 10:90 such that the first component and the second component combined have a polyisobutylene mixture comprising 5.5% of the first medium molecular weight polyisobutylene, 75.6% of the second medium molecular weight polyisobutylene, 16.65% of the first high molecular weight polyisobutylene, and 2.25% of the second high molecular weight polyisobutylene, wherein the matrix further comprises crospovidone in an amount of 23.19% by weight based on a total weight of the matrix; dipropylene glycol LO+ in an amount of 0.77% by weight based on a total weight of the matrix; oleyl alcohol in an amount of 3.64% by weight based on a total weight of the matrix; mineral oil in an amount of 12.89% by weight based on a total weight of the matrix.
191. The transdermal system of embodiment 190, wherein the transdermal system delivers 150 mcg of norelgestromin and 17.5 mcg of ethinyl estradiol per day.
192. A transdermal system for delivering a contraceptive to a human patient in need thereof, the transdermal system comprising a backing and a matrix contacting the backing, wherein the matrix comprises ethinyl estradiol and norelgestromin, and the matrix is configured to deliver about 17.5 mcg to about 20 mcg per day of the ethinyl estradiol and about 190 mcg to about 240 mcg per day of the norelgestromin to the human patient during a seven-day wear period.
193. The transdermal system of embodiment 192, wherein the matrix delivers about 200 mcg per day of the norelgestromin.
194. The transdermal system of embodiment 192, wherein the matrix delivers about 240 mcg per day of the norelgestromin.
195. The transdermal system of embodiment 192, wherein the human patient has a body mass index (BMI) of less than 30 kg/m².
196. The transdermal system of embodiment 192, wherein the human patient has a body mass index (BMI) of greater than or equal to 30 kg/m².
197. A transdermal system for delivering a contraceptive to a human patient in need thereof, the transdermal system comprising a backing and a matrix contacting the backing, wherein the matrix comprises ethinyl estradiol and norelgestromin, and the matrix is configured to deliver about 17.5 mcg to about 20 mcg per day of the ethinyl estradiol and about 190 mcg to about 240 mcg per day of the norelgestromin to the human patient during a seven-day wear period, and the matrix further comprises an adhesive comprising a low molecular weight polyisobutylene having an average molecular weight of about 40,000 to about 85,000 and a high molecular weight polyisobutylene having an average molecular weight of about 500,000 to about 1.5 million.
198. The transdermal system of embodiment 197, wherein the transdermal system is configured to be applied to the human patient having a body mass index (BMI) of greater than 25 kg/m².
199. The transdermal system of embodiment 197, wherein the transdermal system is configured to be applied to the human patient having a body mass index (BMI) of greater than or equal to 25 kg/m² and less than 30 kg/m².
200. The transdermal system of embodiment 197, wherein the transdermal system is configured to be applied to the human patient having a body mass index (BMI) of greater than or equal to 30 kg/m².
201. The transdermal system of embodiment 197, wherein the low molecular weight polyisobutylene has an average molecular weight of 70,000 and the high molecular weight polyisobutylene has an average molecular weight of 1,050,000.
202. The transdermal system of embodiment 192, wherein the matrix further comprises an adhesive having a low molecular weight polyisobutylene that has an average molecular weight of 53,000 and a high molecular weight polyisobutylene that has an average molecular weight of 1,550,000.
203. A transdermal system for a human patient, the transdermal system comprising a backing layer, a release liner, and a matrix disposed between the backing layer and the release liner, the matrix comprising norelgestromin, ethinyl estradiol, and an adhesive, the adhesive comprising a first component comprising a medium molecular weight polyisobutylene having an average molecular weight of about 70,000, a first high molecular weight polyisobutylene having an average molecular weight of about 1,050,000 and a second high molecular weight polyisobutylene having an average molecular weight of about 1,550,000; and the adhesive also comprising a second component comprising both a medium molecular weight polyisobutylene having an average molecular weight of about 53,000 and a high molecular weight polyisobutylene having an average molecular weight of about 1,550,000, and the first component and the second component having a weight ratio, wherein the weight ratio of the first component to the second component is about 10:90.
204. The transdermal system of embodiment 203, wherein the transdermal system delivers from about 200 mcg to about 240 mcg of norelgestromin per day and about 17.5 mcg to about 20 mcg of ethinyl estradiol per day to the human patient.
205. The transdermal system of embodiment 203, wherein the matrix is configured to deliver over a period of seven days the norelgestromin to produce an AUC∞ of about 167 ng·hr/mL to about 239 ng·hr/mL, a Cmax of about 1.1 ng/mL to about 1.5 ng/mL and a t½ of about 30 hours to about 37 hours; and deliver over a period of seven days the ethinyl estradiol to produce an AUC∞ of about 4493 pg·hr/mL to about 6707 pg·hr/mL, a Cmax of about 33.8 pg/mL to about 50.1 pg/mL and a t½ of about 18 hours to about 23 hours.
206. The transdermal system of embodiment 203, wherein the matrix is configured to deliver over a period of seven days the norelgestromin to produce an AUC∞ of about 217 ng·hr/mL, a Cmax of about 1.37 ng/mL and a t½ of about 32 hours; and deliver over a period of seven days the ethinyl estradiol to produce an AUC∞ of about 6193 pg·hr/mL, a Cmax of about 45.58 pg/mL and a t½ of about 21 hours.
207. The transdermal system of embodiment 203, wherein the matrix is configured to deliver over a period of seven days the norelgestromin to produce an AUC∞ of about 150 ng·hr/mL to about 183 ng·hr/mL, a Cmax of about 0.93 ng/mL to about 1.13 ng/mL and a t½ of about 27 hours to about 33 hours; and deliver over a period of seven days the ethinyl estradiol to produce an AUC∞ of about 3783 pg·hr/mL to about 4624 pg·hr/mL, a Cmax of about 25.5 pg/mL to about 31.2 pg/mL and a t½ of about 22 hours to about 27 hours.
208. The transdermal system of embodiment 203, wherein the transdermal system has an adhesion rate greater than 90% after 7 days of application.
209. The transdermal system of embodiment 203, wherein the transdermal system is a contraceptive for use in a childbearing human female.
210. The transdermal system of embodiment 203, wherein the transdermal system has an adhesion rate greater than 90% after 7 days of application to a childbearing human female.
211. The transdermal system of embodiment 203, wherein the transdermal system is a contraceptive and has a Pearl Index between 1.0 to less than 5.0.
212. The transdermal system of embodiment 203, wherein the transdermal system is a contraceptive and has a Pearl Index of 1.5 to 4.18.
213. The transdermal system of embodiment 203, wherein the matrix delivers about 200 mcg per day of the norelgestromin.
214. The transdermal system of embodiment 203, wherein the transdermal system is configured to be applied to the human patient that has a body mass index (BMI) of greater than 25 kg/m².
215. The transdermal system of embodiment 203, wherein the transdermal system is configured to be applied to the human patient that has a body mass index (BMI) of greater than or equal to 25 kg/m² and less than 30 kg/m².
216. The transdermal system of embodiment 203, wherein the transdermal system is configured to be applied to the patient that has a body mass index (BMI) of greater than or equal to 30 kg/m².
217. A transdermal system for a human patient, the transdermal system comprising a backing layer, a release liner, and a matrix disposed between the backing layer and the release liner, the matrix comprising norelgestromin, ethinyl estradiol, and an adhesive, the matrix being configured to deliver about 17.5 mcg to about 20 mcg per day of the ethinyl estradiol and about 190 mcg to about 240 mcg per day of the norelgestromin to the human patient during a seven-day wear period, the adhesive comprising a polyisobutylene mixture comprising 5.5 wt % of a first medium molecular weight polyisobutylene having an average molecular weight of about 70,000, 75.6 wt % of a second medium molecular weight polyisobutylene having an average molecular weight of about 53,000, 16.65 wt % of a first high molecular weight polyisobutylene having an average molecular weight of about 1,550,000, and 2.25 wt % of a second high molecular weight polyisobutylene having an average molecular weight of about 1,050,000 based on a total weight of the adhesive, wherein the matrix further comprises crospovidone in an amount of 23.19 wt %, dipropylene glycol LO+ in an amount of 0.77 wt %, oleyl alcohol in an amount of 3.64 wt %, and mineral oil in an amount of 12.89 wt % by weight based on a total weight of the matrix.
218. The transdermal system of embodiment 217, wherein the transdermal system is a contraceptive and has a Pearl Index of about 4.5 for the human patient having a body mass index (BMI) of less than 25 kg/m².
219. The transdermal system of embodiment 217, wherein the transdermal system is a contraceptive and has a Pearl Index of about 3.75 for the human patient having a body mass index (BMI) of greater than 25 and less than 30 kg/m².
220. The transdermal system of embodiment 217, wherein the transdermal system is a contraceptive and has a Pearl Index of about 4.85 for the human patient having a body mass index (BMI) of greater than or equal to 30 kg/m².
221. The transdermal system of any one of embodiments 192-220, wherein the transdermal system is in patch form.
222. A transdermal system comprising a backing layer, a release liner, and a matrix disposed between the backing layer and the release liner, the matrix comprising norelgestromin, ethinyl estradiol, and an adhesive, the adhesive comprising a first component comprising a medium molecular weight polyisobutylene having an average molecular weight of about 70,000, a first high molecular weight polyisobutylene having an average molecular weight of about 1,050,000 and a second high molecular weight polyisobutylene having an average molecular weight of about 1,550,000; and the adhesive also comprising a second component comprising both a medium molecular weight polyisobutylene having an average molecular weight of about 53,000 and a high molecular weight polyisobutylene having an average molecular weight of about 1,550,000, and the first component and the second component having a weight ratio, wherein the weight ratio of the first component to the second component is about 10:90.
223. The transdermal system of embodiment 222, wherein the transdermal system delivers about 190 mcg to about 240 mcg of norelgestromin per day and about 17.5 mcg to about 20 mcg of ethinyl estradiol per day.
224. The transdermal system of embodiment 222, wherein the matrix is configured to release over a period of seven days the norelgestromin to produce an AUC∞ of about 167 ng·hr/mL to about 239 ng·hr/mL, a Cmax of about 1.1 ng/mL to about 1.5 ng/mL and a t½ of about 30 hours to about 37 hours; and release over a period of seven days the ethinyl estradiol to produce an AUC∞ of about 4493 pg·hr/mL to about 6707 pg·hr/mL, a Cmax of about 33.8 pg/mL to about 50.1 pg/mL and a t½ of about 18 hours to about 23 hours.
225. The transdermal system of embodiment 222, wherein the matrix is configured to release over a period of seven days the norelgestromin to produce an AUC∞ of about 217 ng·hr/mL, a Cmax of about 1.37 ng/mL and a t½ of about 32 hours; and release over a period of seven days the ethinyl estradiol to produce an AUC∞ of about 6193 pg·hr/mL, a Cmax of about 45.58 pg/mL and a t½ of about 21 hours.
226. The transdermal system of embodiment 222, wherein the matrix is configured to release over a period of seven days the norelgestromin to produce an AUC∞ of about 150 ng·hr/mL to about 183 ng·hr/mL, a Cmax of about 0.93 ng/mL to about 1.13 ng/mL and a t½ of about 27 hours to about 33 hours; and release over a period of seven days the ethinyl estradiol to produce an AUC∞ of about 3783 pg·hr/mL to about 4624 pg·hr/mL, a Cmax of about 25.5 pg/mL to about 31.2 pg/mL and a t½ of about 22 hours to about 27 hours.
227. The transdermal system of embodiment 222, wherein the transdermal system has an adhesion rate greater than 90% after 7 days of application.
228. The transdermal system of embodiment 222, wherein the transdermal system has an adhesion rate greater than 90% after 7 days of application to a childbearing human female.
229. The transdermal system of embodiment 222, wherein the transdermal system is a contraceptive and has a Pearl Index of 1.5 to 4.18.
230. A transdermal system comprising a backing layer, a release liner, and a matrix disposed between the backing layer and the release liner, the matrix comprising norelgestromin and ethinyl estradiol; and an adhesive, the adhesive comprising a first component comprising about 55 wt % of a first polyisobutylene having an average molecular weight of about 70,000, about 22.5 wt % of a second polyisobutylene having an average molecular weight of about 1,050,000, and about 22.5 wt % of a third polyisobutylene having an average molecular weight of about 1,550,000; and a second component comprising about 84 wt % of a fourth polyisobutylene having an average molecular weight of about 53,000 and about 16 wt % of the third polyisobutylene, wherein the first component and the second component are present in a weight ratio of about 10:90, such that the combined adhesive comprises a polyisobutylene mixture comprising about 5.5 wt % of the first polyisobutylene, about 75.6 wt % of the fourth polyisobutylene, about 16.65 wt % of the second polyisobutylene, and about 2.25 wt % of the third polyisobutylene, based on the total weight of the mixture.
231. The transdermal system of embodiment 230, wherein (i) the matrix is configured to release over a period of seven days the norelgestromin to produce an AUC∞ of about 167 ng·hr/mL to about 239 ng·hr/mL, a Cmax of about 1.1 ng/mL to about 1.5 ng/mL and a t½ of about 30 hours to about 37 hours; (ii) the matrix is configured to release over a period of seven days the ethinyl estradiol to produce an AUC∞ of about 4493 pg·hr/mL to about 6707 pg·hr/mL, a Cmax of about 33.8 pg/mL to about 50.1 pg/mL and a t½ of about 18 hours to about 23 hours; (iii) the matrix is configured to release over a period of seven days the norelgestromin to produce an AUC∞ of about 217 ng·hr/mL, a Cmax of about 1.37 ng/mL and a t½ of about 32 hours; or (iv) the matrix is configured to release over a period of seven days the ethinyl estradiol to produce an AUC∞ of about 6193 pg·hr/mL, a Cmax of about 45.58 pg/mL and a t½ of about 21 hours.
232. The transdermal system of embodiment 230, wherein the transdermal system delivers about 190 mcg to about 240 mcg of norelgestromin per day and about 17.5 mcg to about 20 mcg of ethinyl estradiol per day.
233. The transdermal system of embodiment 230, wherein the transdermal system is a contraceptive for use in a childbearing human female.
234. The transdermal system of embodiment 230, wherein the transdermal system is a contraceptive and has a Pearl Index between 1.0 to less than 5.0.
235. A transdermal system comprising a backing layer, a release liner, and a matrix disposed between the backing layer and the release liner, the matrix comprising norelgestromin, ethinyl estradiol, and an adhesive, the adhesive comprising a polyisobutylene mixture comprising 5.5 wt % of a first medium molecular weight polyisobutylene having an average molecular weight of about 70,000, 75.6 wt % of a second medium molecular weight polyisobutylene having an average molecular weight of about 53,000, 16.65 wt % of a first high molecular weight polyisobutylene having an average molecular weight of about 1,550,000, and 2.25 wt % of a second high molecular weight polyisobutylene having an average molecular weight of about 1,050,000 based on a total weight of the adhesive, wherein the matrix further comprises crospovidone in an amount of 23.19 wt %, dipropylene glycol LO+ in an amount of 0.77 wt %, oleyl alcohol in an amount of 3.64 wt %, and mineral oil in an amount of 12.89 wt % by weight based on a total weight of the matrix.
236. The transdermal system of embodiment 235, wherein the transdermal system delivers about 190 mcg to about 240 mcg of norelgestromin and about 17.5 mcg to about 20 mcg of ethinyl estradiol per day.
237. A transdermal system comprising a backing layer, a release liner, and a matrix disposed between the backing layer and the release liner, the matrix comprising norelgestromin, ethinyl estradiol, and an adhesive, the adhesive comprising a first component applied to the matrix, and the first component comprising a medium molecular weight polyisobutylene having an average molecular weight of about 70,000, a first high molecular weight polyisobutylene having an average molecular weight of about 1,050,000 and a second high molecular weight polyisobutylene having an average molecular weight of about 1,550,000; and the adhesive also comprising a second component applied to the matrix, the second component comprising both a medium molecular weight polyisobutylene having an average molecular weight of about 53,000 and a high molecular weight polyisobutylene having an average molecular weight of about 1,550,000, and the first component and the second component having a weight ratio, wherein the weight ratio of the first component to the second component is about 10:90.
238. The transdermal system of any one of embodiments 222-237, wherein the transdermal system is in patch form.

Although the invention has been described with reference to embodiments, persons skilled in the art will recognize that changes may be made in form and detail without departing from the spirit and scope of the disclosure.

1.-161. (canceled)

162. A transdermal system comprising a backing layer, a release liner, and a matrix disposed between the backing layer and the release liner, the matrix comprising norelgestromin, ethinyl estradiol, and an adhesive, the adhesive comprising a first component comprising a medium molecular weight polyisobutylene having an average molecular weight of about 70,000, a first high molecular weight polyisobutylene having an average molecular weight of about 1,050,000 and a second high molecular weight polyisobutylene having an average molecular weight of about 1,550,000; and the adhesive also comprising a second component comprising both a medium molecular weight polyisobutylene having an average molecular weight of about 53,000 and a high molecular weight polyisobutylene having an average molecular weight of about 1,550,000, and the first component and the second component having a weight ratio, wherein the weight ratio of the first component to the second component is about 10:90.

163. The transdermal system of claim 162, wherein the transdermal system delivers about 190 mcg to about 240 mcg of norelgestromin per day and about 17.5 mcg to about 20 mcg of ethinyl estradiol per day.

164. The transdermal system of claim 162, wherein the matrix is configured to release over a period of seven days the norelgestromin to produce an AUC∞ of about 167 ng·hr/mL to about 239 ng·hr/mL, a Cmax of about 1.1 ng/mL to about 1.5 ng/mL and a t½ of about 30 hours to about 37 hours; and release over a period of seven days the ethinyl estradiol to produce an AUC∞ of about 4493 pg·hr/mL to about 6707 pg·hr/mL, a Cmax of about 33.8 pg/mL to about 50.1 pg/mL and a t½ of about 18 hours to about 23 hours.

165. The transdermal system of claim 162, wherein the matrix is configured to release over a period of seven days the norelgestromin to produce an AUC∞ of about 217 ng·hr/mL, a Cmax of about 1.37 ng/mL and a t½ of about 32 hours; and release over a period of seven days the ethinyl estradiol to produce an AUC∞ of about 6193 pg·hr/mL, a Cmax of about 45.58 pg/mL and a t½ of about 21 hours.

166. The transdermal system of claim 162, wherein the matrix is configured to release over a period of seven days the norelgestromin to produce an AUC∞ of about 150 ng·hr/mL to about 183 ng·hr/mL, a Cmax of about 0.93 ng/mL to about 1.13 ng/mL and a t½ of about 27 hours to about 33 hours; and release over a period of seven days the ethinyl estradiol to produce an AUC∞ of about 3783 pg·hr/mL to about 4624 pg·hr/mL, a Cmax of about 25.5 pg/mL to about 31.2 pg/mL and a t½ of about 22 hours to about 27 hours.

167. The transdermal system of claim 162, wherein the transdermal system has an adhesion rate greater than 90% after 7 days of application.

168. The transdermal system of claim 162, wherein the transdermal system has an adhesion rate greater than 90% after 7 days of application to a childbearing human female.

169. The transdermal system of claim 162, wherein the transdermal system is a contraceptive and has a Pearl Index of 1.5 to 4.18.

170. A transdermal system comprising a backing layer, a release liner, and a matrix disposed between the backing layer and the release liner, the matrix comprising norelgestromin and ethinyl estradiol; and an adhesive, the adhesive comprising a first component comprising about 55 wt % of a first polyisobutylene having an average molecular weight of about 70,000, about 22.5 wt % of a second polyisobutylene having an average molecular weight of about 1,050,000, and about 22.5 wt % of a third polyisobutylene having an average molecular weight of about 1,550,000; and a second component comprising about 84 wt % of a fourth polyisobutylene having an average molecular weight of about 53,000 and about 16 wt % of the third polyisobutylene, wherein the first component and the second component are present in a weight ratio of about 10:90, such that the combined adhesive comprises a polyisobutylene mixture comprising about 5.5 wt % of the first polyisobutylene, about 75.6 wt % of the fourth polyisobutylene, about 16.65 wt % of the second polyisobutylene, and about 2.25 wt % of the third polyisobutylene, based on the total weight of the mixture.

171. The transdermal system of claim 170, wherein (i) the matrix is configured to release over a period of seven days the norelgestromin to produce an AUC∞ of about 167 ng·hr/mL to about 239 ng·hr/mL, a Cmax of about 1.1 ng/mL to about 1.5 ng/mL and a t½ of about 30 hours to about 37 hours; (ii) the matrix is configured to release over a period of seven days the ethinyl estradiol to produce an AUC∞ of about 4493 pg·hr/mL to about 6707 pg·hr/mL, a Cmax of about 33.8 pg/mL to about 50.1 pg/mL and a t½ of about 18 hours to about 23 hours; (iii) the matrix is configured to release over a period of seven days the norelgestromin to produce an AUC∞ of about 217 ng·hr/mL, a Cmax of about 1.37 ng/mL and a t½ of about 32 hours; or (iv) the matrix is configured to release over a period of seven days the ethinyl estradiol to produce an AUC∞ of about 6193 pg·hr/mL, a Cmax of about 45.58 pg/mL and a t½ of about 21 hours.

172. The transdermal system of claim 170, wherein the transdermal system delivers about 190 mcg to about 240 mcg of norelgestromin per day and about 17.5 mcg to about 20 mcg of ethinyl estradiol per day.

173. The transdermal system of claim 170, wherein the transdermal system is a contraceptive for use in a childbearing human female.

174. The transdermal system of claim 170, wherein the transdermal system is a contraceptive and has a Pearl Index between 1.0 to less than 5.0.

175. A transdermal system comprising a backing layer, a release liner, and a matrix disposed between the backing layer and the release liner, the matrix comprising norelgestromin, ethinyl estradiol, and an adhesive, the adhesive comprising a polyisobutylene mixture comprising 5.5 wt % of a first medium molecular weight polyisobutylene having an average molecular weight of about 70,000, 75.6 wt % of a second medium molecular weight polyisobutylene having an average molecular weight of about 53,000, 16.65 wt % of a first high molecular weight polyisobutylene having an average molecular weight of about 1,550,000, and 2.25 wt % of a second high molecular weight polyisobutylene having an average molecular weight of about 1,050,000 based on a total weight of the adhesive, wherein the matrix further comprises crospovidone in an amount of 23.19 wt %, dipropylene glycol LO+ in an amount of 0.77 wt %, oleyl alcohol in an amount of 3.64 wt %, and mineral oil in an amount of 12.89 wt % by weight based on a total weight of the matrix.

176. The transdermal system of claim 175, wherein the transdermal system delivers about 190 mcg to about 240 mcg of norelgestromin and about 17.5 mcg to about 20 mcg of ethinyl estradiol per day.

177. A transdermal system comprising a backing layer, a release liner, and a matrix disposed between the backing layer and the release liner, the matrix comprising norelgestromin, ethinyl estradiol, and an adhesive, the adhesive comprising a first component applied to the matrix, and the first component comprising a medium molecular weight polyisobutylene having an average molecular weight of about 70,000, a first high molecular weight polyisobutylene having an average molecular weight of about 1,050,000 and a second high molecular weight polyisobutylene having an average molecular weight of about 1,550,000; and the adhesive also comprising a second component applied to the matrix, the second component comprising both a medium molecular weight polyisobutylene having an average molecular weight of about 53,000 and a high molecular weight polyisobutylene having an average molecular weight of about 1,550,000, and the first component and the second component having a weight ratio, wherein the weight ratio of the first component to the second component is about 10:90.
